SEQUENCE LISTING (33 sequences)

SEQ ID NO: 1 - DNA, 3423 bp, Homo sapiens
  source 1..3423: mol_type = other DNA
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 2 - DNA, 3648 bp, Homo sapiens
  source 1..3648: mol_type = other DNA
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 3 - DNA, 3591 bp, Homo sapiens
  source 1..3591: mol_type = other DNA
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 4 - DNA, 3572 bp, Homo sapiens
  source 1..3572: mol_type = other DNA
aaatagcacagccttgctgtgcgtggtagaagttgggttagtgttgacatgctgttgactcaccctcccgaggatggaagctctggcctgggtcaagttgtggtcactgcagttaacagtttgttgatctcagggagtattccacagttgctgatgtaattgacaatgattggagccagctcttccccagattcaaatggaccaattagaggacttgttggttctgtttatcaactatgtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 5 - DNA, 3255 bp, Homo sapiens
  source 1..3255: mol_type = other DNA
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 6 - DNA, 3480 bp, Homo sapiens
  source 1..3480: mol_type = other DNA
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 7 - DNA, 3310 bp, Homo sapiens
  source 1..3310: mol_type = other DNA
gtcggatgattcaagctcacggggacgagcaggagcgctctcgacttttctagagcctcagcgtcctaggactcacctttccctgatcctgcaccgtccctctcctggccccagactctccctcccactgttcacgaagcccaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 8 - DNA, 3594 bp, Homo sapiens
  source 1..3594: mol_type = other DNA
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 9 - DNA, 3537 bp, Homo sapiens
  source 1..3537: mol_type = other DNA
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 10 - DNA, 3369 bp, Homo sapiens
  source 1..3369: mol_type = other DNA
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 11 - DNA, 1795 bp, Homo sapiens
  source 1..1795: mol_type = other DNA
gtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttggtaagtaagctgttc

SEQ ID NO: 12 - DNA, 555 bp, Homo sapiens
  source 1..555: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 13 - DNA, 555 bp, Homo sapiens
  source 1..555: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaagatggatgcagaattccgacatgactcaggatatgaagttcatcatcaaaaattggtgttctttgcagaagatgtgggttcaaacaaaggtgcaatcattggactcatagtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 14 - DNA, 258 bp, Homo sapiens
  source 1..258: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcgctacgaaaatccaacctacaagttctttgagcagatgcagaactag

SEQ ID NO: 15 - DNA, 816 bp, Homo sapiens
  source 1..816: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggagcgccacctgtccaagatgcagcagagcggctacgaaaatccaacctacaagttctttgagcagatgcagaactag

SEQ ID NO: 16 - DNA, 684 bp, Homo sapiens
  source 1..684: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttacagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 17 - DNA, 499 bp, Homo sapiens
  source 1..499: mol_type = other DNA
atgctgcccggtttggcactgctcctgctggccgcctggacggctcgggcgctggagatacccactgatggtaatgctggcctgctggctgaaccccagattgccatgttctgtggcagactgaacatgcacatgaatgtccagaatgggaagtgggactcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 18 - DNA, 898 bp, Homo sapiens
  source 1..898: mol_type = other DNA
atgctgcccggtttggcactgctcctgctggccgcctggacggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggtggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 19 - DNA, 680 bp, Homo sapiens
  source 1..680: mol_type = other DNA
atgctgcccggtttggcactgctcctgcagtgggaggagattcaggatgaagttgatgaactgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgtggcagaacggctacgaaaatccaacctacaagttctttgagcagatgcagaactag

SEQ ID NO: 20 - DNA, 899 bp, Homo sapiens
  source 1..899: mol_type = other DNA
atgctgcccggtttggcactgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 21 - DNA, 541 bp, Homo sapiens
  source 1..541: mol_type = other DNA
atgctgcccggtttggcactgctcctgctggccgcctggacagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 22 - DNA, 274 bp, Homo sapiens
  source 1..274: mol_type = other DNA
atgctgcccggtttggcactgctcctgctggccgcctggacggctcgggcgctggaggtacccaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 23 - DNA, 515 bp, Homo sapiens
  source 1..515: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtatatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 24 - DNA, 888 bp, Homo sapiens
  source 1..888: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 25 - DNA, 757 bp, Homo sapiens
  source 1..757: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 26 - DNA, 1879 bp, Homo sapiens
  source 1..1879: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtacgcgcagaacagaaggacagacagcacaccctaaagcatttcgagcatgtgcgcatggtggatcccaagaaagccgctcagatccggtcccaggttatgacactcctccgtgtgatttatgagcgcatgaatcagtctctctccctgctctacaacgtgcctgcagtggccgaggagattcaggatgaagttggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 27 - DNA, 144 bp, Homo sapiens
  source 1..144: mol_type = other DNA
atgctgcccggtttggcactgctcctgctggccgcctggacggctcgggcgctggaggtacccactgatggtaatgctggcctgctggctgaaccccagattgccatgttctgtggcagaaagttctttgagcagatgcagaac

SEQ ID NO: 28 - protein, 6 aa, unidentified
YENPTY

SEQ ID NO: 29 - protein, 4 aa, unidentified
AICD

SEQ ID NO: 30 - DNA, 21 bp, synthetic construct
  source 1..21: mol_type = other DNA
  modified_base 1: mod_base = OTHER; note = 2'-O-methylguanosine-3'-phosphorothioate
  modified_base 2: mod_base = OTHER; note = 2'-O-methylguanosine-3'-phosphorothioate
  modified_base 3: mod_base = OTHER; note = 2'-O-methylcytidine-3'-phosphate
  modified_base 4: mod_base = OTHER; note = 2'-O-methyluridine-3'-phosphate
  modified_base 5: mod_base = OTHER; note = 2'-O-methyladenosine-3'-phosphate
  modified_base 6: mod_base = OTHER; note = 2'-O-hexadecyl-cytidine-3'-phosphate
  modified_base 7: mod_base = OTHER; note = 2'-O-methylguanosine-3'-phosphate
  modified_base 8: mod_base = OTHER; note = 2'-O-methyladenosine-3'-phosphate
  modified_base 10: mod_base = OTHER; note = 2'-O-methyladenosine-3'-phosphate
  modified_base 12: mod_base = OTHER; note = 2'-O-methyluridine-3'-phosphate
  modified_base 13: mod_base = OTHER; note = 2'-O-methylcytidine-3'-phosphate
  modified_base 14: mod_base = OTHER; note = 2'-O-methylcytidine-3'-phosphate
  modified_base 15: mod_base = OTHER; note = 2'-O-methyladenosine-3'-phosphate
  modified_base 16: mod_base = OTHER; note = 2'-O-methyladenosine-3'-phosphate
  modified_base 17: mod_base = OTHER; note = 2'-O-methylcytidine-3'-phosphate
  modified_base 18: mod_base = OTHER; note = 2'-O-methylcytidine-3'-phosphate
  modified_base 19: mod_base = OTHER; note = 2'-O-methyluridine-3'-phosphorothioate
  modified_base 20: mod_base = OTHER; note = 2'-O-methyladenosine-3'-phosphorothioate
  modified_base 21: mod_base = OTHER; note = 2'-O-methyladenosine-3'-phosphate
  misc_feature 1..8: note = RNA
  misc_feature 10: note = RNA
  misc_feature 12..21: note = RNA
  misc_feature 9: note = DNA
  misc_feature 11: note = DNA
ggctacgaaaatccaacctaa

SEQ ID NO: 31 - DNA, 23 bp, synthetic construct
  source 1..23: mol_type = other DNA
  modified_base 1: mod_base = OTHER; note = 2'-O-methyluridine-3'-phosphorothioate 5'-(E)-Vinylphosphonate
  modified_base 2: mod_base = OTHER; note = 2'-deoxy-2'-fluorouridine-3'-phosphorothioate
  modified_base 3: mod_base = OTHER; note = 2'-O-methyladenosine-3'-phosphate
  modified_base 4: mod_base = OTHER; note = 2'-O-methylguanosine-3'-phosphate
  modified_base 5: mod_base = OTHER; note = 2'-O-methylguanosine-3'-phosphate
  modified_base 6: mod_base = OTHER; note = 2'-O-methyluridine-3'-phosphate
  modified_base 7: mod_base = OTHER; note = Thymidine-glycol nucleic acid (GNA), S-Isomer
  modified_base 8: mod_base = OTHER; note = 2'-O-methylguanosine-3'-phosphate
  modified_base 9: mod_base = OTHER; note = 2'-O-methylguanosine-3'-phosphate
  modified_base 10: mod_base = OTHER; note = 2'-O-methyladenosine-3'-phosphate
  modified_base 11: mod_base = OTHER; note = 2'-O-methyluridine-3'-phosphate
  modified_base 13: mod_base = OTHER; note = 2'-O-methyluridine-3'-phosphate
  modified_base 14: mod_base = OTHER; note = 2'-deoxy-2'-fluorouridine-3'-phosphate
  modified_base 15: mod_base = OTHER; note = 2'-O-methylcytidine-3'-phosphate
  modified_base 17: mod_base = OTHER; note = 2'-O-methyluridine-3'-phosphate
  modified_base 18: mod_base = OTHER; note = 2'-O-methyladenosine-3'-phosphate
  modified_base 19: mod_base = OTHER; note = 2'-O-methylguanosine-3'-phosphate
  modified_base 20: mod_base = OTHER; note = 2'-O-methylcytidine-3'-phosphate
  modified_base 21: mod_base = OTHER; note = 2'-O-methylcytidine-3'-phosphorothioate
  modified_base 22: mod_base = OTHER; note = 2'-O-methylguanosine-3'-phosphorothioate
  modified_base 23: mod_base = OTHER; note = 2'-O-methyluridine
  misc_feature 1..11: note = RNA
  misc_feature 13..15: note = RNA
  misc_feature 17..23: note = RNA
  misc_feature 12: note = DNA
  misc_feature 16: note = DNA
ttaggttggattttcgtagccgt

SEQ ID NO: 32 - RNA, 21 nt, synthetic construct
  source 1..21: mol_type = other RNA
ggctacgaaaatccaacctaa

SEQ ID NO: 33 - RNA, 23 nt, synthetic construct
  source 1..23: mol_type = other RNA
  modified_base 7: mod_base = OTHER; note = 5'-methyluridine-3'-phosphate
  modified_base 12: mod_base = OTHER; note = 5'-methyluridine-3'-phosphate
ttaggttggattttcgtagccgt

(19) United States
(12) Patent Application Publication
(10) Pub. No.: US 2026/0284097 A1
(43) Pub. Date: Sep. 24, 2026

(54) Mivelsiran Compositions and Methods of Use Thereof

(71) Applicant: ALNYLAM PHARMACEUTICALS, INC., CAMBRIDGE, MA (US)

(72) Inventors: BRET LEE BOSTWICK, LEXINGTON, MA (US); NUNE MAKAROVA, BELMONT, MA (US); JULIA SHIRVAN, WINCHESTER, MA (US)

(73) Assignee: ALNYLAM PHARMACEUTICALS, INC., CAMBRIDGE, MA (US)

(21) Appl. No.: 19/478,319

(22) PCT Filed: Apr. 25, 2024
(86) PCT No.: PCT/US2024/026262

§ 371 Date: Oct. 24, 2025

Related U.S. Application Data
(60) Provisional application No. 63/498,250, filed on Apr. 25, 2023.
(60) Provisional application No. 63/500,223, filed on May 4, 2023.
(60) Provisional application No. 63/513,802, filed on Jul. 14, 2023.
(60) Provisional application No. 63/592,809, filed on Oct. 24, 2023.
(60) Provisional application No. 63/625,229, filed on Jan. 25, 2024.

(51) Int. Cl. A61K 31/713; A61K 9/00; A61K 9/08; A61P 25/28

(52) U.S. Cl. CPC A61K 31/713; A61K 9/0019; A61K 9/08; A61P 25/28

(57) ABSTRACT

The disclosure relates to double stranded ribonucleic acid (dsRNAi) agents and compositions targeting the APP gene, as well as methods of inhibiting expression of an APP gene and methods of treating subjects having an APP-associated disease or disorder, such as Alzheimer's disease (e.g., early onset Alzheimer's disease), using such dsRNAi agents and compositions.

### CROSS-REFERENCE TO RELATED APPLICATIONS

This application claims the benefit of priority to U.S. Provisional Application No. 63/498,250, filed on Apr. 25, 2023; U.S. Provisional Application No. 63/500,223, filed on May 4, 2023; U.S. Provisional Application No. 63/513,802, filed on Jul. 14, 2023; U.S. Provisional Application No. 63/592,809, filed on Oct. 24, 2023; and U.S. Provisional Application No. 63/625,229, filed on Jan. 25, 2024. The entire contents of the foregoing applications are hereby incorporated herein by reference.

### SEQUENCE LISTING

The instant application contains a Sequence Listing which has been submitted electronically in XML format and is hereby incorporated by reference in its entirety. The XML copy, created on Apr. 19, 2024, is named A108868_1720WO_SL.xml and is 104,303 bytes in size.

### FIELD OF THE INVENTION

The instant disclosure relates generally to APP-targeting RNAi agents and methods.

### BACKGROUND OF THE INVENTION

The amyloid precursor protein (APP) gene encodes an integral membrane protein expressed in neurons and glia. While the primary function of APP is unknown, secretase-cleaved forms of APP—particularly the Aβ cleavage forms of APP, e.g., Aβ(1-42) (aka Aβ42) and Aβ(1-40) (aka Aβ40) commonly found as the predominant protein in amyloid beta plaques—have long been described as associated with the development and progression of Alzheimer's disease (AD) in affected individuals. Indeed, identification of amyloid beta plaques in a subject is necessary for pathological diagnosis of AD. Aβ cleavage forms of APP have been particularly described to play a critical and even causal role in the development of AD-related/associated diseases, such as cerebral amyloid angiopathy (CAA) and early onset familial Alzheimer's disease (EOAD, EOFAD, or eFAD).

Inhibition of the expression and/or activity of APP with an agent that can selectively and efficiently inhibit APP, and thereby block or dampen the production and/or levels of Aβ cleavage forms of APP, would be useful for preventing or treating a variety of APP-associated diseases and disorders, including AD and EOAD, among others.

Current treatment options for APP-associated diseases and disorders are both limited and largely ineffective. Attempts to treat sporadic forms of AD and EOAD have to date proven unsuccessful—for example, all trials of BACE1 (β-secretase) inhibitors for treatment of sporadic AD have thus far failed (Egan et al. The New England Journal of Medicine, 378:1691-1703; Hung and Fu. Journal of Biomedical Science, 24:47) and a number of human γ-secretase inhibitor programs have been halted for toxicity (Selkoe and Hardy. EMBO Molecular Medicine, 8:595-608). A number of Aβ-directed immunotherapies are in various phases of development, with lecanemab having been approved by the FDA. Lecanemab has been shown to have modest efficacy, slowing the rate of progression of the disease by approximately 25%. To date, approved pharmacologic treatments for APP-associated diseases or disorders are directed to treatment of symptoms, not to prevention or cure, and such treatments are of limited efficacy, particularly as APP-associated diseases or disorders advance in an affected individual. Therefore, there is a need for therapies for subjects suffering from APP-associated diseases and disorders, including a particular need for therapies for subjects suffering from EOAD.

### BRIEF SUMMARY OF THE INVENTION

The present disclosure provides RNAi compositions of which effect the RNA-induced silencing complex (RISC)-mediated cleavage of RNA transcripts of an amyloid precursor protein (APP) gene. The APP gene may be within a cell, e.g., a cell within a subject, such as a human. The present disclosure also provides methods of using the RNAi compositions of the disclosure for inhibiting the expression of an APP gene and/or for treating a subject who would benefit from inhibiting or reducing the expression of an APP gene, e.g., a subject suffering or prone to suffering from an APP-associated disease, for example, Alzheimer's disease (AD), e.g., early onset familial Alzheimer's disease (EOAD or EOFAD).

In some embodiments, the RNAi composition comprises the RNAi agent mivelsiran (also called AD-961583 or ALN-APP), that comprises a sense strand having the nucleotide sequence:
5′-gsgscua(Chd)gadAadAuccaaccusasa-3′ (SEQ ID NO: 30) and an antisense strand having the nucleotide sequence:
5′-VPusUfsaggu(Tgn)ggaudTuUfcdGuagccsgsu-3′ (SEQ ID NO: 31),

Mivelsiran may be used as a free acid or sodium salt. As used herein, reference to “mivelsiran” is to be understood as meaning mivelsiran free acid or mivelsiran sodium salt, unless the free acid or sodium salt is expressly indicated.

Where each nucleotide modification as defined herein.

In one aspect, the present disclosure provides a method for inhibiting the expression of amyloid precursor protein (APP) in a subject having an APP-associated disease in need thereof, comprising administering to the subject a fixed dose of about 25 mg to about 1200 mg of mivelsiran, thereby inhibiting the expression of APP in the subject.

In one aspect, the present disclosure provides a method for treating or preventing an APP-associated disease in a subject in need thereof, the method comprising administering to the subject a fixed dose of about 25 mg to about 1200 mg of mivelsiran or a pharmaceutically acceptable salt thereof, thereby treating or preventing an APP-associated disease in a subject.

In some embodiments of the methods provided herein, the subject is a human.

In some embodiments the APP-associated disorder is Alzheimer's disease (AD). In some embodiments, the APP-associated disorder is early-onset Alzheimer's disease (EOAD).

In some embodiments, the subject shows a decrease in the level of sAPPα and/or sAPPβ in the cerebral spinal fluid (CSF) following administration of mivelsiran or the equivalent amount of a pharmaceutically acceptable salt thereof (e.g., sodium salt). In some embodiments, the subject shows an at least about 40% decrease in the level of sAPPα and/or sAPPβ in the cerebral spinal fluid (CSF). In some embodiments, the subject shows about 50% to about 90% decrease in the level of sAPPα and/or sAPPβ in the cerebral spinal fluid (CSF).

In some embodiments, the subject shows the decrease in the level of sAPPα and/or sAPPβ in the CSF that is sustained for at least 3 months following administration of the fixed dose of mivelsiran or the equivalent amount of a pharmaceutically acceptable salt thereof (e.g., sodium salt). In some embodiments, the subject shows an at least about 40% decrease in the level of sAPPα and/or sAPPβ in the CSF that is sustained for at least 3 months. In some embodiments, the subject shows an at least about 40% decrease in the level of sAPPα and/or sAPPβ in the CSF that is sustained for at least 6 months. In some embodiments, the subject shows at least about 40% decrease in the level of sAPPα and/or sAPPβ in the CSF that is sustained for at least 10 months. In some embodiments, the subject shows an at least about 55% decrease in the level of sAPPα and/or sAPPβ in the CSF that is sustained for at least 3 months. In some embodiments, the subject shows an at least about 55% decrease in the level of sAPPα and/or sAPPβ in the CSF that is sustained for at least 6 months. In some embodiments, the subject shows at least about 55% decrease in the level of sAPPα and/or sAPPβ in the CSF that is sustained for at least 10 months. In some embodiments, the subject shows at least about 20% decrease in the level of sAPPα and/or sAPPβ in the CSF that is sustained for at least 12 months. In some embodiments, the administration of mivelsiran decreases Aβ accumulation in the subject.

In some embodiments, the administration of mivelsiran decreases intracellular and/or extracellular Aβ40 and/or Aβ42 in the subject. In some embodiments, the administration of mivelsiran to the subject decreases amyloid plaque formation and/or accumulation in the subject.

In some embodiments, the subject shows a decrease in a level of a disease biomarker in a body fluid of the subject following administration of the fixed dose mivelsiran or the equivalent amount of a pharmaceutically acceptable salt thereof (e.g., sodium salt). In some embodiments, the body fluid is a cerebrospinal fluid (CSF) or a plasma.

In some embodiments, the body fluid is the CSF, and the disease biomarker in the CSF that is decreased is selected from the group consisting of Aβ40, Aβ42, neurofilament light chain protein (NfL), neurofilament heavy chain protein (NfH), apolipoprotein E (APOE), neurogranin, synaptosome associated protein 25 (SNAP25), S100 calcium binding protein B (S100B), t-Tau, and p-Tau. In some embodiments, the disease biomarker in the CSF that is decreased is Aβ42 and/or Aβ40. In some embodiments, the subject shows an at least about 30% decrease in the level of Aβ42 and/or at least about 50% decrease in the level of Aβ40 in the CSF following administration of mivelsiran or the equivalent amount of a pharmaceutically acceptable salt thereof (e.g., sodium salt). In some embodiments, the subject shows at least about 40% decrease in the level of Aβ42 and/or at least about 60% decrease in the level of Aβ40 in the CSF following administration of mivelsiran or the equivalent amount of a pharmaceutically acceptable salt thereof (e.g., sodium salt).

In some embodiments, the body fluid is the plasma, and the disease biomarker in the plasma of that is decreased is selected from the group consisting of sAPPα, sAPPβ, Aβ40, Aβ42, NfL, t-Tau, and p-Tau.

In some embodiments, neurotoxicity or neuroinflammation is not produced or increased following administration of mivelsiran or the equivalent amount of a pharmaceutically acceptable salt thereof (e.g., sodium salt).

In some embodiments, a level of an inflammatory biomarker is not increased following administration of mivelsiran or the equivalent amount of a pharmaceutically acceptable salt thereof (e.g., sodium salt). In some embodiments, the inflammatory biomarker is selected from the group consisting of complement component 3 (C3), complement component 5 (C5), interleukin-6 (IL-6), interleukin-1 (IL-1), tumor necrosis factor (TNF), monocyte chemoattractant protein-1 (MCP-1), and chitinase-3-like protein 1 (YKL-40).

In some embodiments, the decrease or the increase is relative to a reference level. In some embodiments, the reference level is the level observed in the subject prior to administration of the dsRNA agent, or the level observed in a subject administered a placebo.

In some embodiments, the reference level is a predetermined threshold level. In some embodiments, the reference level is a level observed in age- and matched non-diseased control population.

In some embodiments, the administration of mivelsiran improves one or more signs and/or symptoms of the APP-associated disorder in the subject. In some embodiments, the administration of mivelsiran improves the subject's score in one or more of Repeatable Battery for the Assessment of Neuropsychological Status (RBANS), Alzheimer's Disease Assessment Scale-Cognitive Subscale 13-item Version (ADAS-Cog13), Clinical Dementia Rating (CDR), Neuropsychiatric Inventory Questionnaire (NPI-Q), Pittsburgh Sleep Quality Index (PSQI), and the Alzheimer's Disease Cooperative Study-Clinical Global Impression of Change (ADCS-CGIC).

Dosages of mivelsiran described herein is provided on a free-acid basis unless stated otherwise. In specific embodiments, as shown in FIG. 1, the formula weight of free acid mivelsiran and mivelsiran sodium (also referred to as mivelsiran sodium salt) is 14,849.30 Da and 15,816.50 Da, respectively. Accordingly, in specific embodiments, 100 mg of free acid mivelsiran is equivalent to approximately 106.51 mg of mivelsiran sodium. In some embodiments, the fixed dose of mivelsiran (free acid) is 25 mg to 75 mg, 30 mg to 75 mg, 35 mg to 75 mg, 40 mg to 75 mg, 45 mg to 75 mg, 50 mg to 75 mg, 25 mg to 60 mg, 30 mg to 60 mg, 35 mg to 60 mg, 40 mg to 60 mg, 45 mg to 60 mg, 50 mg to 60 mg, 25 mg to 50 mg, 30 mg to 50 mg, 35 mg to 50 mg, 40 mg to 50 mg, 45 mg to 50 mg, 25 mg to 100 mg, 50 mg to 100 mg, 75 mg to 100 mg, 25 mg to 150 mg, 30 mg to 150 mg, 35 mg to 150 mg, 40 mg to 150 mg, 45 mg to 150 mg, 50 mg to 150 mg, 75 mg to 150 mg, 100 mg to 150 mg, 50 mg to 75 mg, 75 mg to 100 mg, 100 mg to 150 mg, 150 mg to 225 mg, 225 mg to 600 mg, 600 mg to 900 mg, or 900 mg to 1200 mg, or an equivalent amount of mivelsiran salt (e.g., mivelsiran sodium). In some embodiments, the fixed dose is about 25 mg to about 100 mg or about 50 mg to about 100 mg of mivelsiran (free acid) or an equivalent amount of mivelsiran salt (e.g., mivelsiran sodium). In some embodiments, the fixed dose is about 75 mg to about 100 mg of mivelsiran (free acid), or an equivalent amount of mivelsiran salt (e.g., mivelsiran sodium). In some embodiments, the fixed dose is about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, or about 125 mg of mivelsiran (free acid), or an equivalent amount of mivelsiran salt (e.g., mivelsiran sodium).

In some embodiments, the fixed dose is about 30 mg of mivelsiran (free acid), or an equivalent amount of mivelsiran salt (e.g., mivelsiran sodium). In some embodiments, the fixed dose is about 35 mg of mivelsiran (free acid), or an equivalent amount of mivelsiran salt (e.g., mivelsiran sodium). In some embodiments, the fixed dose is about 40 mg of mivelsiran (free acid), or an equivalent amount of mivelsiran salt (e.g., mivelsiran sodium). In some embodiments, the fixed dose is about 45 mg of mivelsiran (free acid), or an equivalent amount of mivelsiran salt (e.g., mivelsiran sodium). In some embodiments, the fixed dose is about 50 mg of mivelsiran (free acid), or an equivalent amount of mivelsiran salt (e.g., mivelsiran sodium). In some embodiments, the fixed dose is about 55 mg of mivelsiran (free acid), or an equivalent amount of mivelsiran salt (e.g., mivelsiran sodium). In some embodiments, the fixed dose is about 60 mg of mivelsiran (free acid), or an equivalent amount of mivelsiran salt (e.g., mivelsiran sodium). In some embodiments, the fixed dose is about 65 mg of mivelsiran (free acid), or an equivalent amount of mivelsiran salt (e.g., mivelsiran sodium). In some embodiments, the fixed dose is about 70 mg of mivelsiran (free acid), or an equivalent amount of mivelsiran salt (e.g., mivelsiran sodium). In some embodiments, the fixed dose is about 75 mg of mivelsiran (free acid), or an equivalent amount of mivelsiran salt (e.g., mivelsiran sodium). In some embodiments, the fixed dose is about 80 mg of mivelsiran (free acid), or an equivalent amount of mivelsiran salt (e.g., mivelsiran sodium). In some embodiments, the fixed dose is about 85 mg of mivelsiran (free acid), or an equivalent amount of mivelsiran salt (e.g., mivelsiran sodium). In some embodiments, the fixed dose is about 90 mg of mivelsiran (free acid), or an equivalent amount of mivelsiran salt (e.g., mivelsiran sodium). In some embodiments, the fixed dose is about 95 mg of mivelsiran (free acid), or an equivalent amount of mivelsiran salt (e.g., mivelsiran sodium). In some embodiments, the fixed dose is about 100 mg of mivelsiran (free acid), or an equivalent amount of mivelsiran salt (e.g., mivelsiran sodium). In some embodiments, the fixed dose is about 125 mg of mivelsiran (free acid), or an equivalent amount of mivelsiran salt (e.g., mivelsiran sodium). In some embodiments, the fixed dose is about 150 mg of mivelsiran (free acid), or an equivalent amount of mivelsiran salt (e.g., mivelsiran sodium).

In some embodiments, the dsRNA agent or salt thereof is administered to the subject as a single dose.

In some embodiments, multiple doses of the fixed dose of mivelsiran is administered to the subject. In some embodiments, a cumulative annual dose of mivelsiran (free acid) is 450 mg or less. In some embodiments, a cumulative annual dose of mivelsiran (free acid) is 360 mg or less. In some embodiments, each dose in the multiple dose regimen is administered at least 3 months apart. In some embodiments, each dose in the multiple dose regimen is administered at least 6 months apart. In some embodiments, each dose in the multiple dose regimen is administered at least 9 months apart. In some embodiments, each dose in the multiple dose regimen is administered at least 12 months apart.

In some embodiments, mivelsiran is administered to the subject intrathecally.

In some embodiments, the subject, before administration of mivelsiran, is positive for amyloid by PET upon administration of an amyloid PET agent, optionally wherein the amyloid PET agent is 18F-labeled. In some embodiments, the CSF of the subject, before administration of mivelsiran: (i) is abnormal for Aβ42, total tau (t-Tau), and phosphorylated tau (p Tau); (ii) has an abnormal p-Tau/Aβ42 ratio or Aβ42 to p-tau index (PTI); or (iii) has an abnormal t tau/Aβ42 ratio or Aβ42 to t-tau index (ATI). In some embodiments, the subject, before administration of mivelsiran, scores greater than 20 on the Mini Mental State Examination (MMSE). In some embodiments, the subject, before administration of mivelsiran, has a CDR Global score of 0.5 or 1.0. In some embodiments, the subject, before administration of mivelsiran, has a clinical diagnosis of mild cognitive impairment or mild dementia due to Alzheimer's disease, and/or has an onset of Alzheimer's disease at an age younger than 65 years.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIG. 1 schematically depicts mivelsiran (also called ALN-APP), which is the RNAi agent AD-961583 that has been modified as shown. mivelsiran is used in the clinical trial described in the examples. In the scheme, where the letters are presented in the form of “Bx,” “B” indicates nucleobase (A, G, C, or U) and “x” indicates modification. Where x is null, it refers to 2′-H modification (“H”); where x is “m,” it refers to a 2′-O-methyl modification (“OMe”); where x is “f,” it refers to a 2′-fluoro modification (“F”); where x is “hd,” it refers to a 2′-O-n-hexadecyl modification (“O-(n-C₁₆H₃₃)”). “S-GNA” refers to an S-glycol nucleic acid modification.

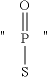

refers to a phosphorothioate internucleotide linkage.

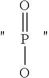

refers to phosphodiester linkages and the 3′-end of each strand terminates in an unmodified 3′-OH group.

FIG. 2 depicts mean leukocyte counts in the CSF following a single dose intrathecal administration of ALN-APP (25 mg, 50 mg, or 75 mg) or placebo on day 0. The reference line indicates 5×10⁶/L leukocytes in the CFS based on average upper limit for healthy individuals across the five central labs used for assessment.

FIG. 3 depicts mean protein concentrations in the CSF following a single dose intrathecal administration of mivelsiran (25 mg, 50 mg, or 75 mg) or placebo on day 0. The reference line indicates 552 mg/L protein in the CFS based on average upper limit for healthy individuals across the five central labs used for assessment.

FIG. 4 depicts median % change from baseline of soluble amyloid alpha precursor protein (sApPα) in cerebrospinal fluid (CSF) following a single dose intrathecal administration of mivelsiran (25 mg, 35 mg, 50 mg, 75 mg, or 100 mg) or placebo on day 0.

FIG. 5 depicts median % change from baseline of soluble amyloid beta precursor protein (sAPPβ) in CSF following a single dose intrathecal administration of mivelsiran (25 mg, 35 mg, 50 mg, 75 mg, or 100 mg) or placebo on day 0.

FIGS. 6A-6C depict mean % change (FIG. 6A), median % change (FIG. 6B), and mean (ng/L) (FIG. 6C) from baseline of an exploratory biomarker Aβ42 in CSF following a single dose intrathecal administration of mivelsiran (25 mg, 35 mg, 50 mg, 75 mg, or 100 mg) or placebo on day 0.

FIGS. 7A-7C depict mean % change (FIG. 7A), median % change (FIG. 7B), and mean (ng/L) (FIG. 7C) from baseline of an exploratory biomarker Aβ40 in CSF following a single dose intrathecal administration of mivelsiran (25 mg, 35 mg, 50 mg, 75 mg, or 100 mg) or placebo on day 0.

FIGS. 8A-8B depict mean % change (FIG. 8A) and median % change (FIG. 8B) from baseline of an exploratory biomarker Aβ42 in plasma following a single dose intrathecal administration of mivelsiran (25 mg, 50 mg, or 75 mg) or placebo on day 0.

FIGS. 9A-9B depict mean % change (FIG. 9A) and median % change (FIG. 9B) from baseline of an exploratory biomarker Aβ40 in plasma following a single dose intrathecal administration of mivelsiran (25 mg, 50 mg, or 75 mg) or placebo on day 0.

### DETAILED DESCRIPTION OF THE INVENTION

The present disclosure provides RNAi compositions, which effect the RNA-induced silencing complex (RISC)-mediated cleavage of RNA transcripts of an amyloid precursor protein (APP) gene. The APP gene may be within a cell, e.g., a cell within a subject, such as a human. The present disclosure also provides methods of using the RNAi compositions of the disclosure for inhibiting the expression of an APP gene and/or for treating a subject having a disorder that would benefit from inhibiting or reducing the expression of an APP gene, e.g., an APP-associated disease, for example, Alzheimer's disease (AD), e.g., early onset familial Alzheimer's disease (EOAD, EOFAD).

The present study evaluates an intrathecally (IT) administered synthetic small interfering RNA (siRNA) designed to lower the levels of amyloid precursor protein (APP) messenger RNA (mRNA). The siRNA agent, referred to herein as mivelsiran, also known as mivelsiran, is being developed in patients with Alzheimer's disease (AD).

mivelsiran is a synthetic siRNA conjugated to a hexadecyl (C16) group for enhanced central nervous system (CNS) delivery. Scientific evidence generated over the last 3 decades implicates amyloid beta (Aβ) aggregation as an early event in the pathogenesis of AD. There is increasing evidence that, in addition to the extracellular Aβ deposition in the brain parenchyma, intracellular Aβ accumulation inside neurons is an early toxic event driving the cascade leading to neurodegeneration (Hardy and Selkoe (2002) Science, 297(5580):353-356). Mutations that lead to increased production of Aβ peptides result in AD, including Early-onset Alzheimer's disease (EOAD). The mivelsiran siRNA is intended to comprehensively lower all intracellular and extracellular amyloid protein species by silencing APP mRNA to ultimately prevent, slow, or potentially reverse the pathophysiological process of AD.

RNAi is a naturally occurring cellular mechanism for regulation of gene expression, mediated by binding of the antisense siRNA strand to the target mRNA sequence with the RNA-induced silencing complex, followed by mRNA cleavage and subsequent suppression of target protein synthesis.

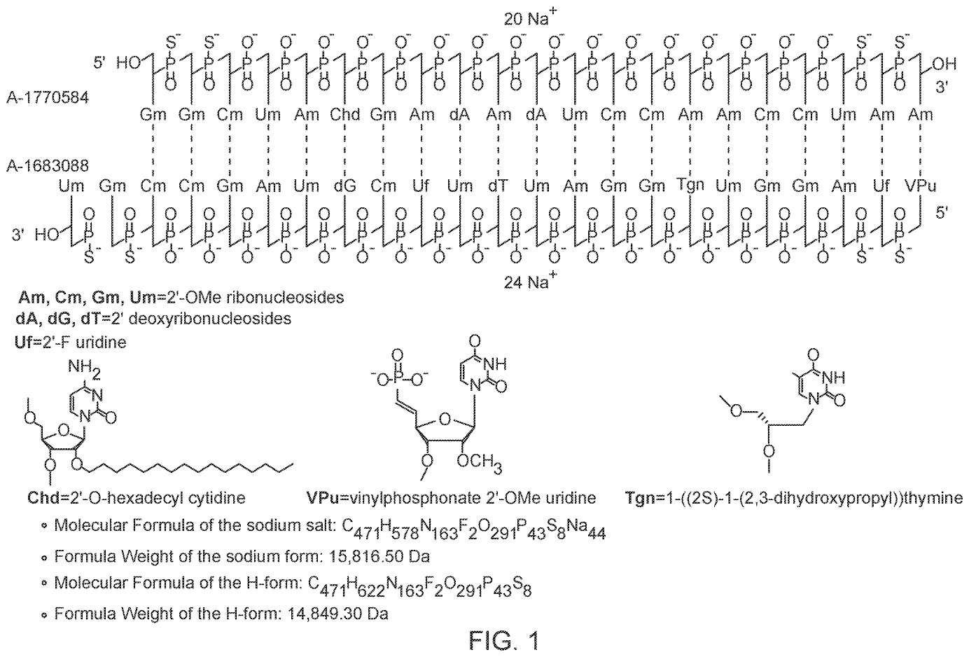

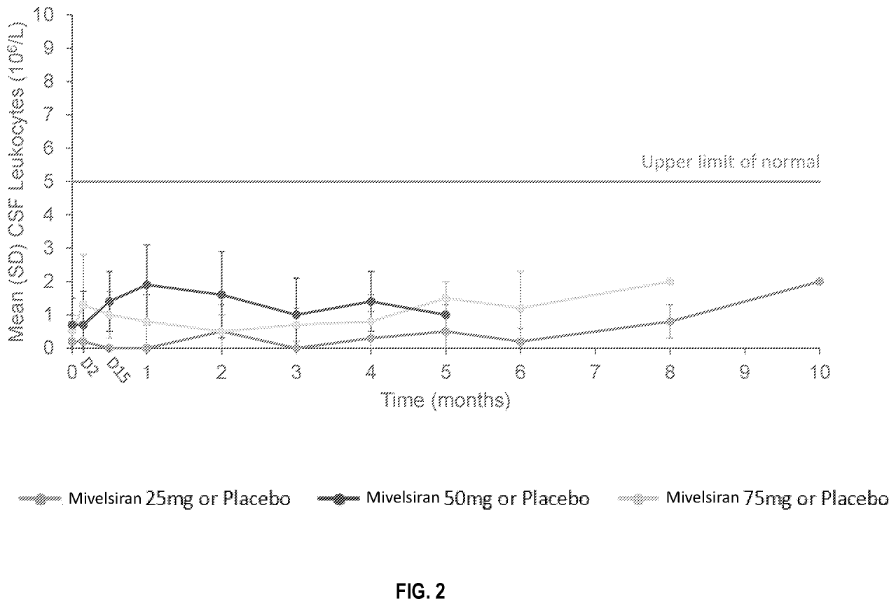

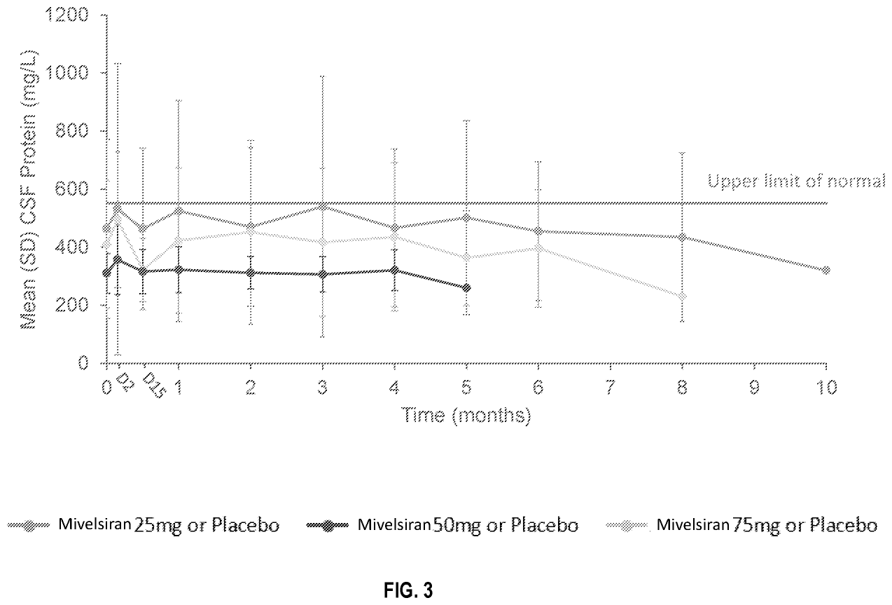

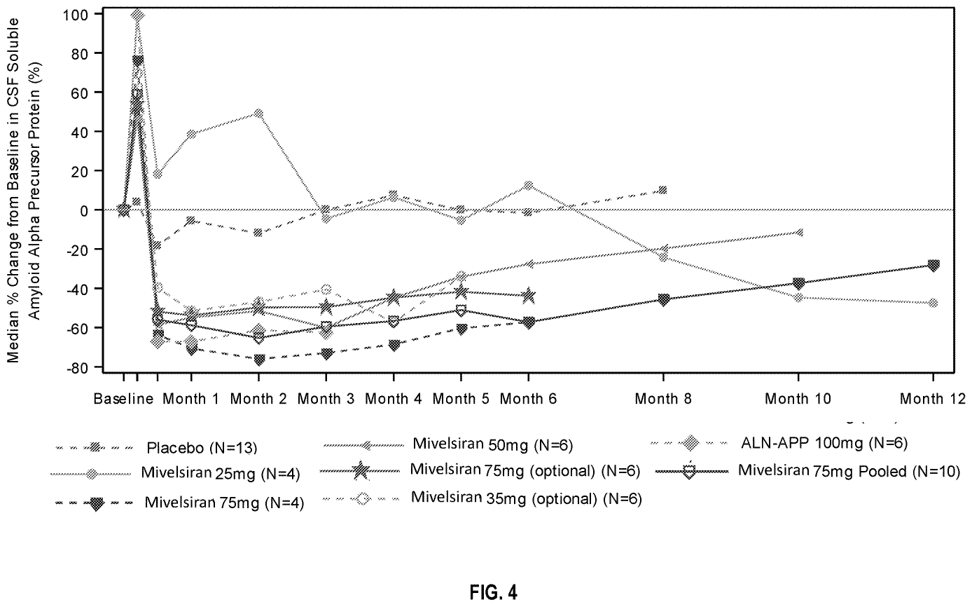

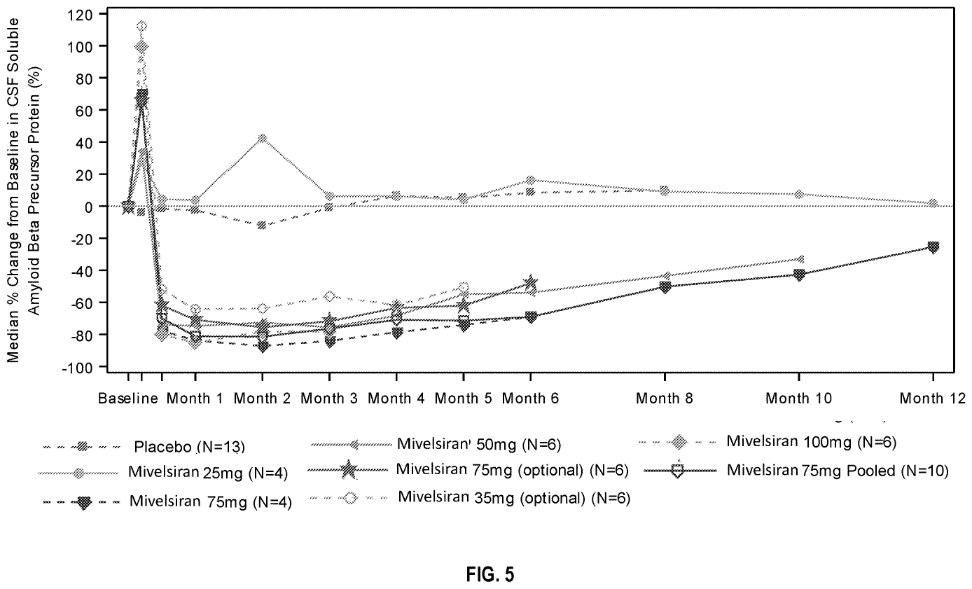

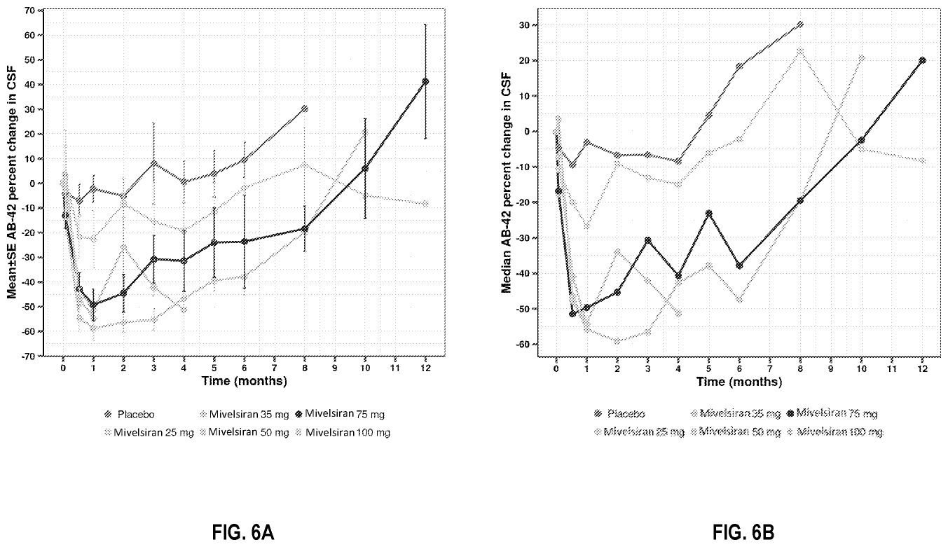

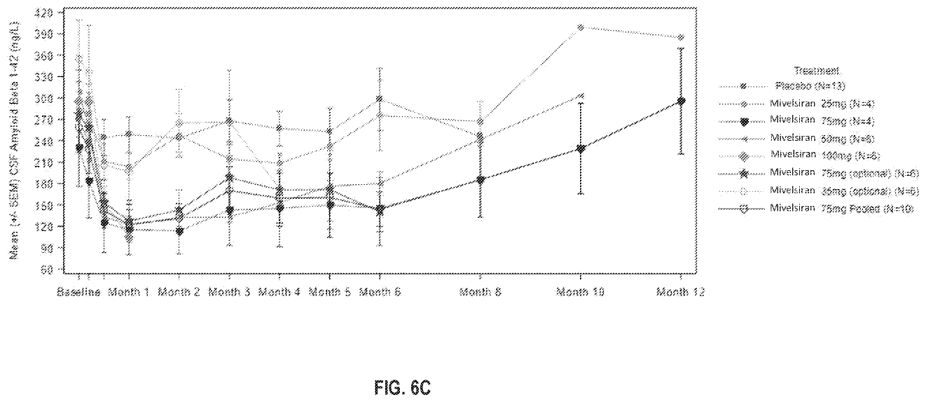

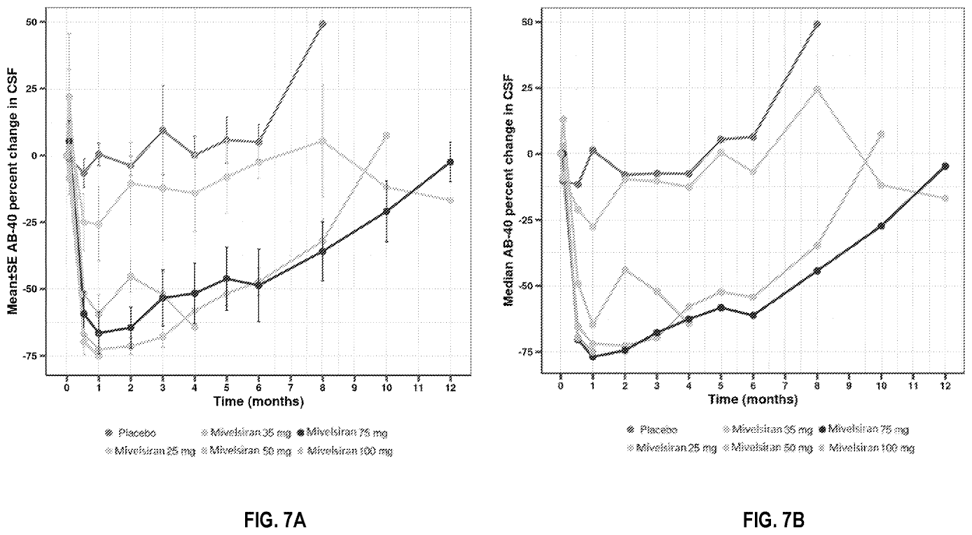

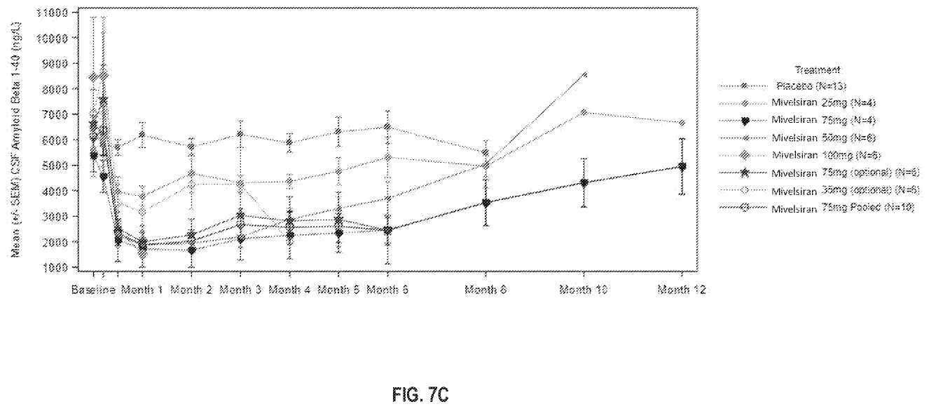

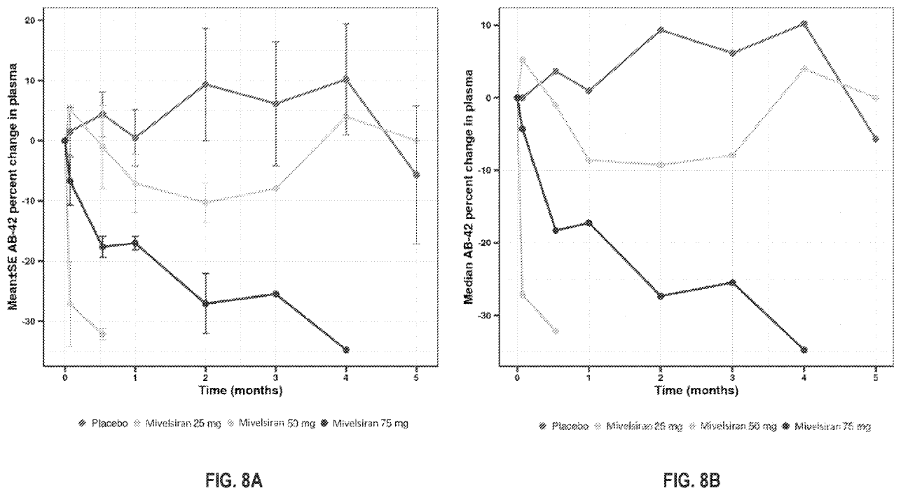

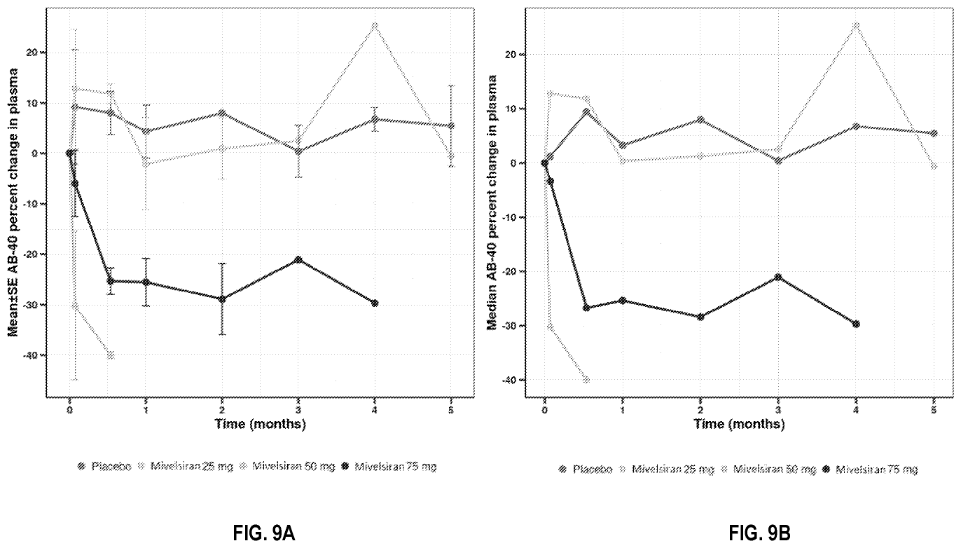

mivelsiran contains an siRNA (AD-961583) targeting APP mRNA, that is conjugated to a hexadecyl (C16) group to facilitate delivery to the CNS. The structure of mivelsiran is shown in FIG. 1.

mivelsiran offers a novel mechanism to treat AD-type pathophysiology by inhibiting the synthesis of APP, the proximate substrate for all post-translational Aβ protein species including Aβ peptide length 40 amino acids (Aβ40) and Aβ peptide length 42 amino acids (Aβ42). Aβ40 and Aβ42 are the primary components of amyloid plaques, an early hallmark of Alzheimer's neuropathology. Thus, reducing Aβ40 and Aβ42 production at the source is expected to lower the substrate for brain amyloid deposition and alter the cascade of pathological events that result in neurodegeneration, resulting in the slowing, halting, or potential reversal of EOAD symptom progression.

The unique mechanism of action of mivelsiran as an siRNA targeting APP mRNA, which results in APP synthesis inhibition, has the potential to confer several advantages over therapeutics which have been studied in AD. mivelsiran targets intracellular APP mRNA transcript, and silences all known APP mutant sequences as well as wild type APP mRNA. Therefore, mivelsiran prevents synthesis of the potentially pathogenic protein rather than attempting to clear the protein once it has been produced. By preventing the translation of APP mRNA into APP protein, mivelsiran acts upstream of the proteolytic process. As a result, it is not expected to interfere with enzymatic processing of other substrates of APP-cleaving enzymes like gamma secretase or beta-site amyloid precursor protein cleaving enzymes (BACE) or result in accumulation of non-Aβ APP cleavage products by inhibiting these enzymes. Unlike antibody-mediated approaches which primarily target Aβ40/42 in the extracellular space, mivelsiran is expected to reduce both intracellular (cell-autonomous) and extracellular (cell non-autonomous) Aβ40/42. Intra-neuronal Aβ oligomers are significant early drivers of AD and act intracellularly to cause neuronal dysfunction (Friedrich et al. (2010) Proc. Natl. Acad. Sci. U.S.A., 107(5):1942-1947; LaFerla et al. (2007) Nat. Rev. Neurosci. 8(7):499-509). Intracellular Aβ oligomers also trigger the formation of neurofibrillary tangles which consists primarily of tau protein, an intraneuronal pathology that is the second hallmark lesion in AD (Bayer and Wirths (2010) Front Aging Neurosci., 2:8; Zhang et al. (2020) Sci. Transl. Med., 12(526):eaay6931). Lastly, Aβ40/42 serve as basic building blocks for dozens of Aβ assembly states, which when combined with numerous post-translational modifications yield hundreds of Aβ protein isoforms (Benilova et al. (2012) Nat. Neurosci., 15(3):349-357). mivelsiran engages the sole precursor of all Aβ peptides and is therefore expected to comprehensively lower all Aβ protein isoforms by acting upstream of protein synthesis. By lowering APP production, mivelsiran may alter the cascade of events that result in neurodegeneration, potentially slowing, halting, or reversing Alzheimer's disease progression.

The use of these RNAi agents enables the targeted degradation of mRNAs of an APP gene in mammals. Very low dosages of APP RNAi agents, in particular, can specifically and efficiently mediate RNA interference (RNAi), resulting in significant inhibition of expression of an APP gene. Using cell-based assays, the present inventors have demonstrated that RNAi agents targeting APP can mediate RNAi, resulting in significant inhibition of expression of an APP gene. Thus, methods and compositions including these RNAi agents are useful for treating a subject who would benefit by a reduction in the levels and/or activity of an APP protein, such as a subject having an APP-associated disease, for example, AD, including, e.g., EOAD.

The following detailed description discloses how to make and use compositions containing RNAi agents to inhibit the expression of an APP gene, as well as compositions and methods for treating subjects having diseases and disorders that would benefit from inhibition and/or reduction of the expression of this gene.

### I. Definitions

In order that the present disclosure may be more readily understood, certain terms are first defined. In addition, it should be noted that whenever a value or range of values of a parameter are recited, it is intended that values and ranges intermediate to the recited values are also intended to be part of this disclosure.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”. The term “or” is used herein to mean, and is used interchangeably with, the term “and/or,” unless context clearly indicates otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

“Mivelsiran”, “AD-961583” and “ALN-APP” each refer to the double-stranded RNA having a sense strand of the nucleotide sequence:
5′-gsgscua(Chd)gadAadAuccaaccusasa-3′ (SEQ ID NO: 30),
   and an antisense strand having the nucleotide sequence:
5′-VPusUfsaggu(Tgn)ggaudTuUfcdGuagccsgsu-3′ (SEQ ID NO: 31),
   wherein
a is 2′-O-methyladenosine-3′-phosphate;
as is 2′-O-methyladenosine-3′-phosphorothioate,
c is a 2′-O-methylcytidine-3′-phosphate;
(Chd) is 2′-O-hexadecyl cytidine-3′-phosphate;
Cs is a 2′-O-methylcytidine-3′-phosphorothioate;
dA is 2′-deoxyadenosine-3-phosphate,
dT is 2′-deoxythymidine-3′-phosphate,
dG is 2′-deoxyguanosine-3-phosphate,
(Tgn) is Thymidine-glycol nucleic acid (GNA)S-Isomer, g
g is a 2′-O-methylguanosine-3′-phosphate;
gs is a 2′-O-methylguanosine-3′-phosphorothioate;
u is a 2′-O-methyluridine-3′-phosphate;
Uf is 2′-fluorouridine-3′-phosphate,
Ufs is 2′-fluorouridine-3′-phosphorothioate,
us is a 2′-O-methyluridine-3′-phosphorothioate; and
VP is vinyl-phosphonate (i.e., “VPus” is 5′-(E-(dihydroxy)phosphinylmethylidene)-5′-deoxy-2′-O-methyluridine 3′-phosphorothioate).

A full chemical name of mivelsiran (ALN-APP) is all-P-ambo-2′-O-methyl-P-thioguanylyl-(3′→5′)-2′-O-methyl-P-thioguanylyl-(3′→5′)-2′-O-methylcytidylyl-(3′→5′)-2′-O-methyluridylyl-(3′→5′)-2′-O-methyladenylyl-(3′→5′)-2′-O-hexadecylcytidylyl-(3′→5′)-2′-O-methylguanylyl-(3′→5′)-2′-O-methyladenylyl-(3′→5′)-2′-deoxyadenylyl-(3′→5′)-2′-O-methyladenylyl-(3′→5′)-2′-deoxyadenylyl-(3′→5′)-2′-O-methyluridylyl-(3′→5′)-2′-O-methylcytidylyl-(3′→5′)-2′-Omethylcytidylyl-(3′→5′)-2′-O-methyladenylyl-(3′→5′)-2′-O-methyladenylyl-(3′→5′)-2′-O-methylcytidylyl-(3′→5′)-2′-O-methylcytidylyl-(3′→5′)-2′-O-methyl-P-thiouridylyl-(3′→5′)-2′-O-methyl-P-thioadenylyl-(3′→5′)-2′-O-methyladenosine duplex with all-P-ambo-2′-O-methyl-P-thiouridylyl-(5′→3′)-2′-O-methyl-P-thioguanylyl-(5′→3′)-2′-O-methylcytidylyl-(5′→3′)-2′-O-methylcytidylyl-(5′→3′)-2′-O-methylguanylyl-(5′→3′)-2′-O-methyladenylyl-(5′→3′)-2′-O-methyluridylyl-(5′→3′)-2′-deoxyguanylyl-(5′→3′)-2′-O-methylcytidylyl-(5′→3′)-2′-deoxy-2′-fluorouridylyl-(5′→3′)-2′-O-methyluridylyl-(5′→3′)-thymidylyl-(5′→3′)-2′-O-methyluridylyl-(5′→3′)-2′-O-methyladenylyl-(5′→3′)-2′-O-methylguanylyl-(5′→3′)-2′-O-methylguanylyl-(5′→2′)-1-de-beta-Dribofuranosyl-1-[(2S)-2,3-dihydroxypropyl]-5-methyluridylyl-(3′→3′)-2′-O-methyluridylyl-(5′→3′)-2′-O-methylguanylyl-(5′→3′)-2′-O-methylguanylyl-(5′→3′)-2′-O-methyl-P-thioadenylyl-(5′→3′)-2′-deoxy-2′-fluoro-P-thiouridylyl-(5′→4′)-1-de-beta-D-ribofuranosyl-1-{(2R,3R,4R,5R)-5-[(1E)-2-phosphonoethen-1-yl]-4-hydroxy-3-methoxyoxolan-2-yl}uridine.

It is intended and understood by one skilled in the art that throughout the disclosure, reference to “mivelsiran” is inclusive of free acid mivelsiran, as well as a pharmaceutically acceptable salt thereof (e.g., mivelsiran sodium). Likewise, dosage amounts of mivelsiran are provided throughout the present disclosure on a free-acid basis unless stated otherwise, and are intended to include the equivalent amount of a pharmaceutically acceptable salt (e.g., sodium salt) of mivelsiran (i.e., mivelsiran sodium), i.e., the equivalent amount of mivelsiran salt that contains the specified amount of mivelsiran. In specific embodiments, as shown in FIG. 1, the formula weight of free acid mivelsiran and mivelsiran sodium (also referred to as mivelsiran sodium salt) is 14,849.30 Da and 15,816.50 Da, respectively. Accordingly, in specific embodiments, 100 mg of free acid mivelsiran is equivalent to approximately 106.51 mg of mivelsiran sodium.

The term “APP” amyloid precursor protein (APP), also known as amyloid beta precursor protein, Alzheimer disease amyloid protein and cerebral vascular amyloid peptide, among other names, having an amino acid sequence from humans, unless specified otherwise. The term also refers to fragments and variants of native APP that maintain at least one in vivo or in vitro activity of a native APP (including, e.g., the beta-amyloid peptide (1-40), beta-amyloid peptide (1-38) and beta-amyloid peptide (1-42) forms of Aβ peptide, among others), including variants of APP fragments that maintain one or more activities of an APP fragment that are neurotoxic in character (e.g., variant forms of Aβ42 peptide that maintain neurotoxic character are expressly contemplated). The term encompasses full-length unprocessed precursor forms of APP as well as mature forms resulting from post-translational cleavage of the signal peptide. The term also encompasses peptides that derive from APP via further cleavage, including, e.g., Aβ peptides. The nucleotide and amino acid sequence of a human APP can be found at, for example, GenBank Accession No. GI: 228008405 (NM_201414; SEQ ID NO: 1). The nucleotide and amino acid sequence of a human APP may also be found at, for example, GenBank Accession No. GI: 228008403 (NM_000484.3; SEQ ID NO: 2); GenBank Accession No. GI: 228008404 (NM_201413.2; SEQ ID NO: 3); GenBank Accession No. GI: 324021746 (NM_001136016.3; SEQ ID NO: 4); GenBank Accession No. GI: 228008402 (NM_001136129.2; SEQ ID NO: 5); GenBank Accession No. GI: 228008401 (NM_001136130.2; SEQ ID NO: 6); GenBank Accession No. GI: 324021747 (NM_001136131.2; SEQ ID NO: 7); GenBank Accession No. GI: 324021737 (NM_001204301.1; SEQ ID NO: 8); GenBank Accession No. GI: 324021735 (NM_001204302.1; SEQ ID NO: 9); and GenBank Accession No. GI: 324021739 (NM_001204303.1; SEQ ID NO: 10); and GenBank Accession No. GI: 1370481385 (XM_024452075.1; SEQ ID NO: 11).

The term “APP” as used herein also refers to a particular polypeptide expressed in a cell by naturally occurring DNA sequence variations of the APP gene, such as a single nucleotide polymorphism in the APP gene. Numerous SNPs within the APP gene have been identified and may be found at, for example, NCBI dbSNP (see, e.g., www.ncbi.nlm.nih.gov/snp). Non-limiting examples of SNPs within the APP gene may be found at, NCBI dbSNP Accession Nos. rs193922916, rs145564988, rs193922916, rs214484, rs281865161, rs364048, rs466433, rs466448, rs532876832, rs63749810, rs63749964, rs63750064, rs63750066, rs63750151, rs63750264, rs63750363, rs63750399, rs63750445, rs63750579, rs63750643, rs63750671, rs63750734, rs63750847, rs63750851, rs63750868, rs63750921, rs63750973, rs63751039, rs63751122 and rs63751263. Certain exemplary rare APP variants that have been previously described to play a role in development of EOAD were identified in Hooli et al. (Neurology 78:1250-57). In addition, various “non-classical” APP variants that harbor an intraexonic junction within sequenced cDNA have recently been identified as associated with the occurrence of somatic gene recombination in the brains of AD patients. Examples of such “non-classical” APP variants include cAPP-R3/16 (SEQ ID NO: 12), cAPP-R3/16-2 (SEQ ID NO: 13), cAPP-R2/18 (SEQ ID NO: 14), cAPP-R6/18 (SEQ ID NO: 15), cAPP-R3/14 (SEQ ID NO: 16), cAPP-R3/17 (SEQ ID NO: 17), cAPP-R1/11 (SEQ ID NO: 18), CAPP-R1/13 (SEQ ID NO: 19), cAPP-R1/11-2 (SEQ ID NO: 20), cAPP-R1/14 (SEQ ID NO: 21), cAPP-R2/17 (SEQ ID NO: 22), cAPP-R2/16 (SEQ ID NO: 23), cAPP-R6/17 (SEQ ID NO: 24), cAPP-R2/14 (SEQ ID NO: 25), cAPP-R14/17-d8 (SEQ ID NO: 26) and cAPP-D2/18-3 (SEQ ID NO: 27). It is expressly contemplated that mivelsiran (ALN-APP) can be used to target “non-classical” APP variants. Such “non-classical” APP variants were described as notably absent from an assayed HIV patient population, with prevalence of AD in the HIV patient population significantly diminished as compared to expected levels, which indicated that reverse transcriptase inhibitors and/or other anti-retroviral therapies commonly used to treat HIV patients likely also exerted a therapeutic/preventative role against AD. It is therefore expressly contemplated that mivelsiran (ALN-APP) can optionally be employed in combination with reverse transcriptase inhibitors and/or other anti-retroviral therapies, for therapeutic and/or preventative purposes.

In one embodiment, a “RNAi agent” for use in the compositions and methods of the disclosure is a double stranded RNA and is referred to herein as a “double stranded RNAi agent,” “double stranded RNA (dsRNA) molecule,” “dsRNA agent,” or “dsRNA”. The term “dsRNA” refers to a complex of ribonucleic acid molecules, having a duplex structure comprising two anti-parallel and substantially complementary nucleic acid strands, referred to as having “sense” and “antisense” orientations with respect to a target RNA, i.e., an APP gene.

The term “antisense strand” or “guide strand” refers to the strand of a RNAi agent, e.g., a dsRNA, which includes a region that is substantially complementary to a target sequence, e.g., an APP mRNA.

The term “sense strand” or “passenger strand” as used herein, refers to the strand of a RNAi agent that includes a region that is substantially complementary to a region of the antisense strand as that term is defined herein.

The phrase “contacting a cell with an RNAi agent,” such as a dsRNA, as used herein, includes contacting a cell by any possible means. Contacting a cell with an RNAi agent includes contacting a cell in vitro with the RNAi agent or contacting a cell in vivo with the RNAi agent. The contacting may be done directly or indirectly. Thus, for example, the RNAi agent may be put into physical contact with the cell by the individual performing the method, or alternatively, the RNAi agent may be put into a situation that will permit or cause it to subsequently come into contact with the cell.

Contacting a cell in vitro may be done, for example, by incubating the cell with the RNAi agent. Contacting a cell in vivo may be done, for example, by injecting the RNAi agent into or near the tissue where the cell is located, or by injecting the RNAi agent into another area, e.g., the central nervous system (CNS), optionally via intrathecal, such that the agent will subsequently reach the tissue where the cell to be contacted is located. For example, the RNAi agent may contain and/or be coupled to a ligand, e.g., a lipophilic moiety or moieties as described below and further detailed, e.g., in U.S. Application Nos. 62/668,072, 62/738,747 and/or 62/773,082, that directs and/or otherwise stabilizes the RNAi agent at a site of interest, e.g., the CNS.

In one embodiment, contacting a cell with a RNAi agent includes “introducing” or “delivering the RNAi agent into the cell” by facilitating or effecting uptake or absorption into the cell. Absorption or uptake of a RNAi agent can occur through unaided diffusive or active cellular processes, or by auxiliary agents or devices. Introducing a RNAi agent into a cell may be in vitro and/or in vivo. For example, for in vivo introduction, a RNAi agent can be injected into a tissue site or administered systemically. In vitro introduction into a cell includes methods known in the art such as electroporation and lipofection. Further approaches are described herein below and/or are known in the art.

As used herein, a “subject” is a human, such as a human being treated or assessed for a disease, disorder or condition that would benefit from reduction in APP expression; a human at risk for a disease, disorder or condition that would benefit from reduction in APP expression; a human having a disease, disorder or condition that would benefit from reduction in APP expression; and/or human being treated for a disease, disorder or condition that would benefit from reduction in APP expression as described herein.

As used herein, the terms “treating” or “treatment” refer to a beneficial or desired result including, but not limited to, alleviation or amelioration of one or more symptoms associated with APP gene expression and/or APP protein production, e.g., APP-associated diseases or disorders such as AD) and EOAD, among others. “Treatment” can also mean prolonging survival as compared to expected survival in the absence of treatment.

The term “lower” in the context of the level of APP in a subject or a disease marker or symptom refers to a statistically significant decrease in such level. The decrease can be, for example, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or more. In certain embodiments, a decrease is at least 20%. “Lower” in the context of the level of APP in a subject is preferably down to a level accepted as within the range of normal for an individual without such disorder.

As used herein, “prevention” or “preventing,” when used in reference to a disease, disorder or condition thereof, that would benefit from a reduction in expression of an APP gene and/or production of APP protein, refers to a reduction in the likelihood that a subject will develop a symptom associated with such a disease, disorder, or condition, e.g., a symptom of APP gene expression, such as the presence of various forms of Aβ (e.g., Aβ38, Aβ43, Aβ40, and/or Aβ42, etc.), amyloid plaques and/or Alzheimer's disease (AD), including, e.g., early onset familial Alzheimer's disease (EOAD, EOFAD). The failure to develop a disease, disorder or condition, or the reduction in the development of a symptom associated with such a disease, disorder or condition (e.g., by at least about 10% on a clinically accepted scale for that disease or disorder), or the exhibition of delayed symptoms delayed (e.g., by days, weeks, months or years) is considered effective prevention.

As used herein, the term “APP-associated disease,” is a disease or disorder that is caused by, or associated with APP gene expression or APP protein production. The term “APP-associated disease” includes a disease, disorder or condition that would benefit from a decrease in APP gene expression, replication, or protein activity. Non-limiting examples of APP-associated diseases include, for example, CAA and Alzheimer's disease (AD), including, e.g., early onset familial Alzheimer's disease (EOAD, EOFAD).

A “statistically significant” increase or decrease in a parameter refers to an increase or decrease in the parameter that is significant as statistically analyzed. Any known statistical methods can be used for statistical analysis and determination of the presence of a statistically significant increase or decrease, including the chi square test, analysis of variance (ANOVA) test (e.g., paired 2 way ANOVA tests with a Sidak Post-hoc multiple comparison test), Student's t-test, multivariate analysis or variance (MANOVA), and analysis of covariance (ANCOVA).

“Therapeutically effective amount,” as used herein, is intended to include the amount of an RNAi agent that, when administered to a subject having an APP-associated disorder, is sufficient to effect treatment of the disease (e.g., by diminishing, ameliorating or maintaining the existing disease or one or more symptoms of disease). The “therapeutically effective amount” may vary depending on the RNAi agent, how the agent is administered, the disease and its severity and the history, age, weight, family history, genetic makeup, the types of preceding or concomitant treatments, if any, and other individual characteristics of the subject to be treated.

“Prophylactically effective amount,” as used herein, is intended to include the amount of a RNAi agent that, when administered to a subject having an APP-associated disorder, is sufficient to prevent or ameliorate the disease or one or more symptoms of the disease. Ameliorating the disease includes slowing the course of the disease or reducing the severity of later-developing disease. The “prophylactically effective amount” may vary depending on the RNAi agent, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount of a RNAi agent that produces some desired local or systemic effect at a reasonable benefit/risk ratio applicable to any treatment. A RNAi agent employed in the methods of the present disclosure may be administered in a sufficient amount to produce a reasonable benefit/risk ratio applicable to such treatment.

The phrase “pharmaceutically acceptable” is employed herein to refer to those compounds, materials, compositions, and/or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; and (22) other non-toxic compatible substances employed in pharmaceutical formulations.

The phrase “pharmaceutically acceptable salt” as used herein refers to any pharmaceutically acceptable chemical compound formed by an acid and a base, with all part of the hydrogen of the acid replaced by a metal or other cation. A pharmaceutically acceptable salt of an RNAi agent (e.g., mivelsiran) as used herein include, but are not limited to, a sodium salt, a calcium salt, a lithium salt, a potassium salt, an ammonium salt, a magnesium salt, or any mixture thereof, of the RNAi agent (e.g., mivelsiran). One skilled in the art will appreciate that the RNAi agent, when provided as a polycationic salt having one cation per free acid group of the optionally modified phosophodiester backbone and/or any other acidic modifications (e.g., 5-terminal phosphonate groups). For example, an oligonucleotide of “n” nucleotides in length contains n−1 optionally modified phosophodiesters, so that an oligonucleotide of 21 nt in length may be provided as a salt having up to 20 cations (e.g., 20 sodium cations). Similarly, an RNAi agents having a sense strand of 21 nt in length and an antisense strand of 23 nt in length may be provided as a salt having up to 42 cations (e.g., 42 sodium cations). In the preceding example, where the RNAi agent also includes a 5′-terminal phosphate or a 5′-terminal vinylphosphonate group, the RNAi agent may be provided as a salt having up to 44 cations (e.g., 44 sodium cations). In specific embodiments, pharmaceutically acceptable salt is a sodium salt, such as mivelsiran sodium.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs or localized regions. For example, samples may be derived from particular organs, parts of organs, or fluids or cells within those organs. In certain embodiments, samples may be derived from the brain (e.g., whole brain or certain segments of brain or certain types of cells in the brain, such as, e.g., neurons and glial cells (astrocytes, oligodendrocytes, microglial cells)). In some embodiments, a “sample derived from a subject” refers to blood or plasma drawn from the subject. In further embodiments, a “sample derived from a subject” refers to brain tissue (or subcomponents thereof) or retinal tissue (or subcomponents thereof) derived from the subject.

### RNAi Agents of the Disclosure

A dsRNA can be synthesized by standard methods known in the art as further discussed below, e.g., by use of an automated DNA synthesizer, such as are commercially available from, for example, Biosearch, Applied Biosystems, Inc. For example, the nucleic acids featured in the disclosure can be synthesized and/or modified by methods well established in the art, such as those described in “Current protocols in nucleic acid chemistry,” Beaucage, S. L. et al. (Edrs.), John Wiley & Sons, Inc., New York, NY, USA.

Mivelsiran may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Single-stranded oligonucleotides of the disclosure can be prepared using solution-phase or solid-phase organic synthesis or both.

In specific embodiments, The RNAi agents of the present disclosure (e.g., mivelsiran, the structure of which shown in FIG. 1) demonstrate surprising and superior results as compared to antisense oligonucleotides that have been tested in human clinical studies. For example, the RNAi agents provided herein demonstrate dose-dependent, rapid, and sustained reductions of expression levels of the target, soluble APPα and APPβ (sAPPα and sAPPβ), in cerebrospinal fluid. At day 15 following a 75 mg single dose administration of mivelsiran, mean reductions from baseline of 55% (sAPPα) and 69% (sAPPβ), with maximum reductions of 71% (sAPPα) and 83% (sAPPβ) can be achieved. Over 4 months following a 75 mg single dose administration of mivelsiran, mean reductions from baseline of about 55-60% (sAPPα) and about 70-80% (sAPPβ), median reductions from baseline of about 60-80% (sAPP) and about 75-85% (sAPPβ), and maximum reductions of 84% (sAPPα) and 90% (sAPPβ) can be produced. The APP dsRNA provided herein can produce median decrease of both sAPPα and sAPPβ of greater than 70% that is sustained for at least 3 months following a single dose intrathecal administration. (see Example 2). The RNAi agents provided herein can achieve these results in effectively inhibiting target expression with only a single dose without a loading dose, whereas loading doses are typically needed with anti-sense oligonucleotides.

Moreover, the RNAi agents provided herein can achieve the target expression knockdown that is greater than has been possible with other CNS-targeted anti-sense oligonucleotides. For example, a knockdown of about 33% at 12 months with a 100 mg dose was observed in a phase 1-2 trial of tofersen, an antisense oligonucleotide targeting superoxide dismutase 1 (SOD1), in amyotrophic lateral sclerosis (ALS) patients (Miller, T, et al. (2020) New England J Med. 383(2):109-119). In contrast, the APP dsRNA agents of the present invention achieved a higher sustained knockdown, maximum reduction of 84% and 90% for sAPPα and sAPPβ, respectively, at a lower dose of 75 mg (see Example 2).

In some embodiments, the APP dsRNA agents provided herein surprisingly have a long knockdown duration of at least 4 months and the knockdown is achieved at lower doses than was expected at the start of the trial. As demonstrated in Example 2, the single dose of 75 mg surprisingly has a sustained knockdown effect of up to 90% on sAPPα and sAPPβ lasting at least out to four months post-administration and the study is still ongoing. Overall, the APP dsRNA agents demonstrate superior properties in dosing, knockdown, and duration compared to other CNS-targeted antisense oligonucleotides that have been tested.

CNS administration of antisense oligonucleotides can result in side effects such as neurotoxicity and neuroinflammation (Hagedorn, P. H. et al. (2022) Nucleic Acid Therapeutics, 32(3):151-162; Goyenvalle, A. et al. (2023) Nucleic Acid Therapeutics, 33(1):1-16). Importantly, in certain embodiments, the RNAi agents provided herein do not produce these side effects when administered to humans to treat APP-associated disease (e.g., Alzheimer's disease, early-onset Alzheimer's disease).

### APP Knockdown to Treat APP-Associated Diseases

Certain aspects of the instant disclosure are directed to RNAi agent-mediated knockdown of APP-associated diseases or disorders, which include AD, including EOAD, as well as sporadic and/or late onset AD. CAA (e.g., hereditary CAA) is another APP-associated disease. Aspects of CAA and RNAi agent-mediated knockdown for treatment of CAA are further discussed, e.g., in WO2020132227, the entire content of which is incorporated herein by reference.

RNAi agent-mediated knockdown of EOAD is expressly contemplated. EOAD is a devastating and rare disease and a causal role of APP is well-established and phenotyping of the disease can be performed with greater accuracy and over a shorter duration of time than, e.g., sporadic and/or late onset AD (optionally late onset AD with severe CAA as a subclass of late onset AD). EOAD is a progressive, dementing neurodegenerative disease in young adults, possessing an age of onset before age 60 to 65 years and often before 55 years of age.

The prevalence of EOAD has been estimated to be 41.2 per 100,000 for the population at risk (i.e., persons aged 40-59 years), with 61% of those affected by EOAD having a positive family history of EOAD (among these, 13% had affected individuals in three generations). EOAD comprises less than 3% of all AD (Bird, Genetics in Medicine, 10:231-239; Brien and Wang. Annu Rev Neu Sci, 2011, 34:185-204; NCBI Gene Reviews).

Providing human genetic validation of the APP target (OMIM 104300), certain APP mutations have been identified that cause EOAD, including E665D, K670N, M671L (Swedish), T714A (Iranian), T714I (Austrian), V715M (French), V715A (German), 1716V (Florida), 1716T, V717I (London), V717F, V717G and V717L, as described above. In addition, dominant amyloid beta precursor protein mutations have also been identified that cause EOAD.

Without wishing to be bound by theory, the pathogenesis of AD is believed to begin in the hippocampus, a ridge of grey matter immediately superior to both lateral ventricles. Degeneration of this tissue is believed to cause the memory loss characteristic of early disease. While the mechanism of neurodegeneration at the protein level has been a matter of great debate, duplications of APP associated with EOAD have indicated that overexpression of APP may be sufficient to cause AD. (Haass and Selkoe. Nature Reviews Molecular Cell Biology, 8:101-112).

In contrast to EOAD, the pathogenic mechanisms of sporadic AD are not yet understood and the population of clinically defined sporadic AD is probably mechanistically heterogeneous.

Certain aspects of the instant disclosure are directed towards targeting of APP for knockdown in individuals having EOAD. A need exists for such agents because only symptom-directed treatments (of limited efficacy) exist for AD more generally and EOAD in particular. In certain embodiments, the RNAi agents of the instant disclosure should provide approximately 60-80% knockdown of both mutant and WT APP levels throughout the CNS. One further observation from human genetics that speaks to the likely therapeutic efficacy of an APP-targeted therapy capable of knocking down APP levels in CNS cells is that an A673T mutation was identified that protected carriers from AD and dementia in the general population (Jonsson et al. Nature Letter, 488. doi:doi:10.1038/nature11283). The A673T substitution is adjacent to a B-secretase cleavage site, and has been described as resulting in a 40% reduction in amyloid beta in cell assays. Thus, a dominant negative APP point mutant appeared to protect families from AD, further reinforcing that RNAi agent-mediated knockdown of APP could exert a similar protective and/or therapeutic effect in at least certain forms of AD, including EOAD.

As noted above, attempts to treat sporadic forms of AD and EOAD have to date proven unsuccessful—for example, all trials of BACE1 (β-secretase) inhibitors (BACE1i) for treatment of sporadic AD have thus far failed (Egan et al. The New England Journal of Medicine, 378:1691-1703; Hung and Fu. Journal of Biomedical Science, 24:47). In such BACEi testing, there have been no completed studies in genetically defined populations (only studies initiated). Notably, the most recent BACE1i study showed that Verubecestat lowered amyloid beta levels by 60% in a population selected based on age and clinical criteria that suggested a probable diagnosis of AD (Egan et al. The New England Journal of Medicine, 378:1691-1703; Hung and Fu. Journal of Biomedical Science, 24:47). Meanwhile, among Aβ-directed immunotherapies, one such immunotherapy demonstrated proof-of-concept in a recent trial in sporadic AD, supporting initiation of an ongoing Phase III trial (Selkoe and Hardy. EMBO Molecular Medicine, 8:595-608). Given its role in APP cleavage, γ-secretase has also been targeted in certain AD-directed trials. However, to date no y-secretase inhibitor trials have been completed in a genetically defined population; and several programs have been discontinued for toxicity (Selkoe and Hardy).

A need therefore exists for agents that can treat or prevent APP-associated diseases or disorders in an affected individual.

The RNAi agents of the present disclosure, by reducing APP protein production, are expected to reduce the secretion of Aβ peptides that aggregate into extracellular amyloid deposits and/or reduce the intraneuronal APP cleavage products in APP-associated diseases or disorders, e.g., AD and EOAD. For example, the RNAi agents provided herein are expected to reduce amyloid deposits in brain tissue, tau tangles in neurons, and neurodegeneration in AD (e.g., EOAD).

It is expressly contemplated that all APP-associated diseases or disorders can ultimately be targeted using the RNAi agents of the instant disclosure, even in view of the diagnostic/phenotyping issues presently confronted for some of the APP-associated diseases (it is further contemplated that diagnostics for these diseases will also continue to improve).

### Delivery of a RNAi Agent of the Disclosure

The delivery of a mivelsiran to a cell e.g., a cell within a subject, such as a human subject (e.g., a subject in need thereof, such as a subject having an APP-associated disorder, e.g., AD, e.g., EOAD) can be achieved in a number of different ways. For example, delivery may be performed by contacting a cell with mivelsiran either in vitro or in vivo. In vivo delivery may also be performed directly by administering a composition comprising a RNAi agent, e.g., a dsRNA, to a subject.

Certain aspects of the instant disclosure relate to a method of reducing the expression of an APP target gene in a cell, comprising contacting said cell with mivelsiran. In one embodiment, the cell is an extrahepatic cell, optionally a CNS cell.

Another aspect of the disclosure relates to a method of reducing the expression of an APP target gene in a subject, comprising administering to the subject mivelsiran.

Another aspect of the disclosure relates to a method of treating a subject having a CNS disorder, comprising administering to the subject a therapeutically effective amount of mivelsiran, thereby treating the subject. Exemplary CNS disorders that can be treated by the method of the disclosure include Alzheimer's, amyotrophic lateral sclerosis (ALS), frontotemporal dementia, Huntington's, Parkinson, spinocerebellar, prion, and lafora.

In one embodiment, mivelsiran is administered intrathecally. By intrathecal administration of mivelsiran, the method can reduce the expression of an APP target gene in a brain or spine tissue, for instance, cortex, cerebellum, striatum, cervical spine, lumbar spine, and thoracic spine.

mivelsiran can be incorporated into pharmaceutical compositions suitable for administration. Such compositions typically include one or more species of RNAi agent and a pharmaceutically acceptable carrier. As used herein the language “pharmaceutically acceptable carrier” is intended to include any and all solvents, dispersion media, antibacterial and antifungal agents, isotonic, and the like, compatible with pharmaceutical administration. The use of such media and agents for pharmaceutically active substances is well known in the art. Except insofar as any conventional media or agent is incompatible with the active compound, use thereof in the compositions is contemplated.

The pharmaceutical compositions of the present disclosure may be administered in a number of ways depending upon whether local or systemic treatment is desired and upon the area to be treated. Administration may be parenteral. Parenteral administration includes intrathecal or intraventricular administration.

Compositions for intrathecal or intraventricular administration may include sterile aqueous solutions which may also contain buffers, diluents and other suitable additives.

Formulations for parenteral administration may include sterile aqueous solutions which may also contain buffers, diluents and other suitable additives. Intraventricular injection may be facilitated by an intraventricular catheter, for example, attached to a reservoir.

Administration can be provided by the subject or by another person, e.g., a health care provider. The medication can be provided in measured doses or in a dispenser which delivers a metered dose. Selected modes of delivery are discussed in more detail below.

Intrathecal Administration. In one embodiment, mivelsiran is delivered by intrathecal injection (i.e., injection into the spinal fluid which bathes the brain and spinal cord tissue). Intrathecal injection of RNAi agents into the spinal fluid can be performed as a bolus injection or via minipumps which can be implanted beneath the skin, providing a regular and constant delivery of siRNA into the spinal fluid. The circulation of the spinal fluid from the choroid plexus, where it is produced, down around the spinal cord and dorsal root ganglia and subsequently up past the cerebellum and over the cortex to the arachnoid granulations, where the fluid can exit the CNS, that, depending upon size, stability, and solubility of the compounds injected, molecules delivered intrathecally could hit targets throughout the entire CNS.

In some embodiments, the intrathecal administration is via a pump. The pump may be a surgically implanted osmotic pump. In one embodiment, the osmotic pump is implanted into the subarachnoid space of the spinal canal to facilitate intrathecal administration.

In some embodiments, the intrathecal administration is via an intrathecal delivery system for a pharmaceutical including a reservoir containing a volume of the pharmaceutical agent, and a pump configured to deliver a portion of the pharmaceutical agent contained in the reservoir.

The amount of intrathecally injected RNAi agents may vary from one target gene to another target gene and the appropriate amount that has to be applied may have to be determined individually for each target gene.

### Pharmaceutical Compositions of the Disclosure

The present disclosure also includes pharmaceutical compositions and formulations which include mivelsiran. In one embodiment, provided herein are pharmaceutical compositions containing a RNAi agent, as described herein, and a pharmaceutically acceptable carrier. The pharmaceutical compositions containing the RNAi agent are useful for treating a disease or disorder associated with the expression or activity of an APP gene, e.g., an APP-associated disease, e.g., AD, e.g., EOAD.

Such pharmaceutical compositions are formulated based on the mode of delivery. One example is compositions that are formulated for systemic administration via parenteral delivery. Another example is compositions that are formulated for direct delivery into the CNS, e.g., by intrathecal injection, optionally by infusion into the brain, such as by continuous pump infusion.

The pharmaceutical compositions of the disclosure may be administered in dosages sufficient to inhibit expression of an APP gene. In general, a suitable dose of a RNAi agent of the disclosure will be in the range of about 0.001 to about 200.0 milligrams per kilogram body weight of the recipient per day, generally in the range of about 1 to 50 mg per kilogram body weight per day. Typically, a suitable dose of a RNAi agent of the disclosure will be in the range of about 0.1 mg/kg to about 5.0 mg/kg, preferably about 0.3 mg/kg and about 3.0 mg/kg.

In one embodiment, the dosing frequency of a therapeutic amount of mivelsiran is a single dose. In further embodiments, the single dose of mivelsiran (free acid) is administered at about 25 mg, 50 mg, 75 mg, 100 mg, 150 mg, 225 mg, 600 mg, or up to a maximum dose of 1200 mg), or an equivalent amount of mivelsiran salt (e.g., mivelsiran sodium). An “equivalent amount” of mivelsiran salt as used herein with respect to a first amount of free acid mivelsiran means a second amount of mivelsiran salt that contains the same number of moles as the first amount of the free acid mivelsiran and/or has the same activity (e.g., bioactivity) as the first amount of free acid mivelsiran. In specific embodiments, the formula weight of free acid mivelsiran and mivelsiran sodium (also referred to as mivelsiran sodium salt) is 14,849.30 Da and 15,816.50 Da, respectively. Accordingly, in specific embodiments, 100 mg of free acid mivelsiran is equivalent to approximately 106.51 mg of mivelsiran sodium. In specific embodiments, mivelsiran provided herein is administered at a single dose of about 50-100 mg or 75-100 mg or 100-150 mg (free acid mivelsiran), or an equivalent amount of mivelsiran salt (e.g., mivelsiran sodium).

In some embodiments, mivelsiran is administered at a single dose of about 30 mg (free acid), or an equivalent amount of mivelsiran salt (e.g., mivelsiran sodium). In some embodiments, mivelsiran is administered at a single dose of about 35 mg (free acid), or an equivalent amount of mivelsiran salt (e.g., mivelsiran sodium). In some embodiments, mivelsiran is administered at a single dose of about 40 mg (free acid), or an equivalent amount of mivelsiran salt (e.g., mivelsiran sodium). In some embodiments, mivelsiran is administered at a single dose of about 45 mg (free acid), or an equivalent amount of mivelsiran salt (e.g., mivelsiran sodium). In some embodiments, mivelsiran is administered at a single dose of about 50 mg (free acid), or an equivalent amount of mivelsiran salt (e.g., mivelsiran sodium). In some embodiments, mivelsiran is administered at a single dose of about 55 mg (free acid), or an equivalent amount of mivelsiran salt (e.g., mivelsiran sodium). In some embodiments, mivelsiran is administered at a single dose of about 60 mg (free acid), or an equivalent amount of mivelsiran salt (e.g., mivelsiran sodium). In some embodiments, mivelsiran is administered at a single dose of about 65 mg (free acid), or an equivalent amount of mivelsiran salt (e.g., mivelsiran sodium). In some embodiments, mivelsiran is administered at a single dose of about 70 mg (free acid), or an equivalent amount of mivelsiran salt (e.g., mivelsiran sodium). In some embodiments, mivelsiran is administered at a single dose of about 75 mg (free acid), or an equivalent amount of mivelsiran salt (e.g., mivelsiran sodium). In some embodiments, mivelsiran is administered at a single dose of about 80 mg (free acid), or an equivalent amount of mivelsiran salt (e.g., mivelsiran sodium). In some embodiments, mivelsiran is administered at a single dose of about 85 mg (free acid), or an equivalent amount of mivelsiran salt (e.g., mivelsiran sodium). In some embodiments, mivelsiran is administered at a single dose of about 90 mg (free acid), or an equivalent amount of mivelsiran salt (e.g., mivelsiran sodium). In some embodiments, mivelsiran is administered at a single dose of about 95 mg (free acid), or an equivalent amount of mivelsiran salt (e.g., mivelsiran sodium). In some embodiments, mivelsiran is administered at a single dose of about 100 mg (free acid), or an equivalent amount of mivelsiran salt (e.g., mivelsiran sodium). In some embodiments, mivelsiran is administered at a single dose of about 125 mg (free acid), or an equivalent amount of mivelsiran salt (e.g., mivelsiran sodium). In some embodiments, mivelsiran is administered at a single dose of about 150 mg (free acid), or an equivalent amount of mivelsiran salt (e.g., mivelsiran sodium).

In some embodiments, mivelsiran is administered in a multiple-dose regimen or repeat-dose regimen. A repeat-dose regimen may include administration of a therapeutic amount of mivelsiran on a regular basis, such as bi-monthly or monthly to once a year. In certain embodiments, mivelsiran is administered about once per month to about once per quarter (i.e., about once every three months).

In some embodiments, after an initial treatment regimen, the treatments can be administered on a less frequent basis.

In some embodiments of the disclosure, a single dose of mivelsiran is administered once per week, bi-monthly, once per month, once every 2 months, once every 3 months, once every 4 months, once every 5 months, once every 6 months, or once every 7 months, or once every 8 months, or once every 9 months, or once every 10 months, or once every 11 months, or once every 12 months. In specific embodiments, a single dose of mivelsiran is administered at least 3 months apart (e.g., every 3 months or less frequently). In further embodiments, a single dose of mivelsiran is administered once every 6 months or less frequently. In further embodiments, a single dose of mivelsiran is administered once every 12 months or less frequently.

In another embodiment, the dosing frequency mivelsiran is a multi-dose regimen. In one embodiment, mivelsiran or pharmaceutically acceptable salt thereof is administered about once every three months. The dose can be repeated every three months for 12 months. In certain embodiments of the multi-dose administration, mivelsiran is administered at about 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 112 mg, 115 mg, 120 mg, 125 mg, 130 mg, 135 mg, 140 mg, 145 mg, 150 mg, or up to a maximum of 225 mg, with no greater than about 450 mg cumulative dose given per annum (e.g., maximum regimens of 225 mg every 6 months) or up to a maximum of 180 mg, with no greater than about 360 mg cumulative dose given per annum (e.g., maximum regimens of 180 mg every 6 months) as free acid mivelsiran, or at an equivalent amount of mivelsiran salt (e.g., mivelsiran sodium). In one embodiment, the RNAi agent of the disclosure is administered no more than once every 3, 4, or 6 months.

In one embodiment, mivelsiran is administered at a dosing regimen of 225 mg (free acid) or an equivalent amount of mivelsiran salt (e.g., mivelsiran sodium), once every 6 months for a total of 12 months. In one embodiment, mivelsiran is administered at a dosing regimen of 180 mg (free acid) or an equivalent amount of mivelsiran salt (e.g., mivelsiran sodium), once every 6 months for a total of 12 months. In one embodiment, mivelsiran is administered at a dosing regimen of 150 mg (free acid) or an equivalent amount of mivelsiran salt (e.g., mivelsiran sodium), once every 4 months for a total of 12 months. In another embodiment, mivelsiran is administered at a dosing regimen of 112 mg (free acid) or an equivalent amount of mivelsiran salt (e.g., mivelsiran sodium), once every 3 months for a total of 12 months.

In one embodiment, mivelsiran is administered at a dosing regimen of 50-100 mg (free acid) or an equivalent amount of mivelsiran salt (e.g., mivelsiran sodium), once every 6 months. In one embodiment, mivelsiran is administered at a dosing regimen of 100 mg (free acid) or an equivalent amount of mivelsiran salt (e.g., mivelsiran sodium), once every 6 months. In one embodiment, mivelsiran is administered at a dosing regimen of 75 mg (free acid) or an equivalent amount of mivelsiran salt (e.g., mivelsiran sodium), once every 6 months. In one embodiment, mivelsiran is administered at a dosing regimen of 50 mg (free acid) or an equivalent amount of mivelsiran salt (e.g., mivelsiran sodium), once every 6 months.

In one embodiment, mivelsiran is administered at a dosing regimen of 50-100 mg (free acid) or an equivalent amount of mivelsiran salt (e.g., mivelsiran sodium) once every 9 months. In one embodiment, the RNAi agent of the disclosure is administered at a dosing regimen of 100 mg (free acid) or an equivalent amount of mivelsiran salt (e.g., mivelsiran sodium) once every 9 months. In one embodiment, mivelsiran is administered at a dosing regimen of 75 mg (free acid) or an equivalent amount of mivelsiran salt (e.g., mivelsiran sodium) once every 9 months. In one embodiment, mivelsiran is administered at a dosing regimen of 50 mg (free acid) or an equivalent amount of mivelsiran salt (e.g., mivelsiran sodium) once every 9 months.

In one embodiment, mivelsiran is administered at a dosing regimen of 50-100 mg (free acid) or an equivalent amount of mivelsiran salt (e.g., mivelsiran sodium) once every 12 months. In one embodiment, the RNAi agent of the disclosure is administered at a dosing regimen of 100 mg (free acid) or an equivalent amount of mivelsiran salt (e.g., mivelsiran sodium) once every 12 months. In one embodiment, mivelsiran is administered at a dosing regimen of 75 mg (free acid) or an equivalent amount of mivelsiran salt (e.g., mivelsiran sodium) once every 12 months. In one embodiment, mivelsiran is administered at a dosing regimen of 50 mg (free acid) or an equivalent amount of mivelsiran salt (e.g., mivelsiran sodium) once every 12 months.

In one embodiment, mivelsiran is administered at a dosing regimen of 30-75 mg (free acid) or an equivalent amount of mivelsiran salt (e.g., mivelsiran sodium) once every 6 months. In one embodiment, mivelsiran is administered at a dosing regimen of 30 mg (free acid) or an equivalent amount of mivelsiran salt (e.g., mivelsiran sodium) once every 6 months. In one embodiment, mivelsiran is administered at a dosing regimen of 75 mg (free acid) or an equivalent amount of mivelsiran salt (e.g., mivelsiran sodium) once every 6 months. In one embodiment, mivelsiran is administered at a dosing regimen of 50 mg (free acid) or an equivalent amount of mivelsiran salt (e.g., mivelsiran sodium) once every 6 months.

In one embodiment, mivelsiran is administered at a dosing regimen of 30-75 mg (free acid) or an equivalent amount of mivelsiran salt (e.g., mivelsiran sodium) once every 9 months. In one embodiment, mivelsiran is administered at a dosing regimen of 30 mg (free acid) or an equivalent amount of mivelsiran salt (e.g., mivelsiran sodium) once every 9 months. In one embodiment, mivelsiran is administered at a dosing regimen of 75 mg (free acid) or an equivalent amount of mivelsiran salt (e.g., mivelsiran sodium) once every 9 months. In one embodiment, mivelsiran is administered at a dosing regimen of 50 mg (free acid) or an equivalent amount of mivelsiran salt (e.g., mivelsiran sodium) once every 9 months.

In one embodiment, mivelsiran is administered at a dosing regimen of 30-75 mg (free acid) or an equivalent amount of mivelsiran salt (e.g., mivelsiran sodium) once every 12 months. In one embodiment, mivelsiran is administered at a dosing regimen of 30 mg (free acid) or an equivalent amount of mivelsiran salt (e.g., mivelsiran sodium) once every 12 months. In one embodiment, mivelsiran is administered at a dosing regimen of 75 mg (free acid) or an equivalent amount of mivelsiran salt (e.g., mivelsiran sodium) once every 12 months. In one embodiment, mivelsiran is administered at a dosing regimen of 50 mg (free acid) or an equivalent amount of mivelsiran salt (e.g., mivelsiran sodium) once every 12 months.

In one embodiment, mivelsiran is administered at a dosing regimen of 30-60 mg (free acid) or an equivalent amount of mivelsiran salt (e.g., mivelsiran sodium) once every 6 months. In one embodiment, mivelsiran is administered at a dosing regimen of 30 mg (free acid) or an equivalent amount of mivelsiran salt (e.g., mivelsiran sodium) once every 6 months. In one embodiment, mivelsiran is administered at a dosing regimen of 60 mg (free acid) or an equivalent amount of mivelsiran salt (e.g., mivelsiran sodium) once every 6 months. In one embodiment, mivelsiran is administered at a dosing regimen of 50 mg (free acid) or an equivalent amount of mivelsiran salt (e.g., mivelsiran sodium) once every 6 months.

In one embodiment, mivelsiran is administered at a dosing regimen of 30-60 mg (free acid) or an equivalent amount of mivelsiran salt (e.g., mivelsiran sodium) once every 9 months. In one embodiment, mivelsiran is administered at a dosing regimen of 30 mg (free acid) or an equivalent amount of mivelsiran salt (e.g., mivelsiran sodium) once every 9 months. In one embodiment, mivelsiran is administered at a dosing regimen of 60 mg (free acid) or an equivalent amount of mivelsiran salt (e.g., mivelsiran sodium) once every 9 months. In one embodiment, mivelsiran is administered at a dosing regimen of 50 mg (free acid) or an equivalent amount of mivelsiran salt (e.g., mivelsiran sodium) once every 9 months.

In one embodiment, mivelsiran is administered at a dosing regimen of 30-60 mg (free acid) or an equivalent amount of mivelsiran salt (e.g., mivelsiran sodium) once every 12 months. In one embodiment, mivelsiran is administered at a dosing regimen of 30 mg (free acid) or an equivalent amount of mivelsiran salt (e.g., mivelsiran sodium) once every 12 months. In one embodiment, mivelsiran is administered at a dosing regimen of 60 mg (free acid) or an equivalent amount of mivelsiran salt (e.g., mivelsiran sodium) once every 12 months. In one embodiment, mivelsiran is administered at a dosing regimen of 50 mg (free acid) or an equivalent amount of mivelsiran salt (e.g., mivelsiran sodium) once every 12 months.

In one embodiment, mivelsiran is administered at a dosing regimen of 30-50 mg (free acid) or an equivalent amount of mivelsiran salt (e.g., mivelsiran sodium) once every 6 months. In one embodiment, mivelsiran is administered at a dosing regimen of 30 mg (free acid) or an equivalent amount of mivelsiran salt (e.g., mivelsiran sodium) once every 6 months. In one embodiment, mivelsiran is administered at a dosing regimen of 40 mg (free acid) or an equivalent amount of mivelsiran salt (e.g., mivelsiran sodium) once every 6 months. In one embodiment, mivelsiran is administered at a dosing regimen of 50 mg once every 6 months.

In one embodiment, mivelsiran is administered at a dosing regimen of 30-50 mg (free acid) or an equivalent amount of mivelsiran salt (e.g., mivelsiran sodium) once every 9 months. In one embodiment, mivelsiran is administered at a dosing regimen of 30 mg (free acid) or an equivalent amount of mivelsiran salt (e.g., mivelsiran sodium) once every 9 months. In one embodiment, mivelsiran is administered at a dosing regimen of 40 mg (free acid) or an equivalent amount of mivelsiran salt (e.g., mivelsiran sodium) once every 9 months. In one embodiment, mivelsiran is administered at a dosing regimen of 50 mg (free acid) or an equivalent amount of mivelsiran salt (e.g., mivelsiran sodium) once every 9 months.

In one embodiment, mivelsiran is administered at a dosing regimen of 30-50 mg (free acid) or an equivalent amount of mivelsiran salt (e.g., mivelsiran sodium) once every 12 months. In one embodiment, mivelsiran is administered at a dosing regimen of 30 mg (free acid) or an equivalent amount of mivelsiran salt (e.g., mivelsiran sodium) once every 12 months. In one embodiment, mivelsiran is administered at a dosing regimen of 40 mg (free acid) or an equivalent amount of mivelsiran salt (e.g., mivelsiran sodium) once every 12 months. In one embodiment, mivelsiran is administered at a dosing regimen of 50 mg (free acid) or an equivalent amount of mivelsiran salt (e.g., mivelsiran sodium) once every 12 months.

In one embodiment, mivelsiran is administered at a dosing regimen of 50-75 mg (free acid) or an equivalent amount of mivelsiran salt (e.g., mivelsiran sodium) once every 6 months. In one embodiment, mivelsiran is administered at a dosing regimen of 60 mg (free acid) or an equivalent amount of mivelsiran salt (e.g., mivelsiran sodium) once every 6 months. In one embodiment, mivelsiran is administered at a dosing regimen of 75 mg (free acid) or an equivalent amount of mivelsiran salt (e.g., mivelsiran sodium) once every 6 months. In one embodiment, mivelsiran is administered at a dosing regimen of 50 mg (free acid) or an equivalent amount of mivelsiran salt (e.g., mivelsiran sodium) once every 6 months.

In one embodiment, mivelsiran is administered at a dosing regimen of 50-75 mg (free acid) or an equivalent amount of mivelsiran salt (e.g., mivelsiran sodium) once every 9 months. In one embodiment, mivelsiran is administered at a dosing regimen of 60 mg (free acid) or an equivalent amount of mivelsiran salt (e.g., mivelsiran sodium) once every 9 months. In one embodiment, mivelsiran is administered at a dosing regimen of 75 mg (free acid) or an equivalent amount of mivelsiran salt (e.g., mivelsiran sodium) once every 9 months. In one embodiment, mivelsiran is administered at a dosing regimen of 50 mg (free acid) or an equivalent amount of mivelsiran salt (e.g., mivelsiran sodium) once every 9 months.

In one embodiment, mivelsiran is administered at a dosing regimen of 50-75 mg (free acid) or an equivalent amount of mivelsiran salt (e.g., mivelsiran sodium) once every 12 months. In one embodiment, mivelsiran is administered at a dosing regimen of 60 mg (free acid) or an equivalent amount of mivelsiran salt (e.g., mivelsiran sodium) once every 12 months. In one embodiment, mivelsiran is administered at a dosing regimen of 75 mg (free acid) or an equivalent amount of mivelsiran salt (e.g., mivelsiran sodium) once every 12 months. In one embodiment, mivelsiran is administered at a dosing regimen of 50 mg (free acid) or an equivalent amount of mivelsiran salt (e.g., mivelsiran sodium) once every 12 months.

In one embodiment, mivelsiran is administered at a dosing regimen of 100-150 mg (free acid) or an equivalent amount of mivelsiran salt (e.g., mivelsiran sodium), once every 6 months. In one embodiment, mivelsiran is administered at a dosing regimen of 100 mg (free acid) or an equivalent amount of mivelsiran salt (e.g., mivelsiran sodium), once every 6 months. In one embodiment, mivelsiran is administered at a dosing regimen of 125 mg (free acid) or an equivalent amount of mivelsiran salt (e.g., mivelsiran sodium), once every 6 months. In one embodiment, mivelsiran is administered at a dosing regimen of 150 mg (free acid) or an equivalent amount of mivelsiran salt (e.g., mivelsiran sodium), once every 6 months.

In one embodiment, mivelsiran is administered at a dosing regimen of 100-150 mg (free acid) or an equivalent amount of mivelsiran salt (e.g., mivelsiran sodium) once every 9 months. In one embodiment, the RNAi agent of the disclosure is administered at a dosing regimen of 100 mg (free acid) or an equivalent amount of mivelsiran salt (e.g., mivelsiran sodium) once every 9 months. In one embodiment, mivelsiran is administered at a dosing regimen of 125 mg (free acid) or an equivalent amount of mivelsiran salt (e.g., mivelsiran sodium) once every 9 months. In one embodiment, mivelsiran is administered at a dosing regimen of 150 mg (free acid) or an equivalent amount of mivelsiran salt (e.g., mivelsiran sodium) once every 9 months.

In one embodiment, mivelsiran is administered at a dosing regimen of 100-150 mg (free acid) or an equivalent amount of mivelsiran salt (e.g., mivelsiran sodium) once every 12 months. In one embodiment, the RNAi agent of the disclosure is administered at a dosing regimen of 100 mg (free acid) or an equivalent amount of mivelsiran salt (e.g., mivelsiran sodium) once every 12 months. In one embodiment, mivelsiran is administered at a dosing regimen of 125 mg (free acid) or an equivalent amount of mivelsiran salt (e.g., mivelsiran sodium) once every 12 months. In one embodiment, mivelsiran is administered at a dosing regimen of 150 mg (free acid) or an equivalent amount of mivelsiran salt (e.g., mivelsiran sodium) once every 12 months.

The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual RNAi agents encompassed by the disclosure can be made using conventional methodologies or on the basis of in vivo testing using an appropriate animal model, as described elsewhere herein.

The pharmaceutical compositions of the present disclosure can be administered in a number of ways depending upon whether local or systemic treatment is desired and upon the area to be treated. Administration can be parenteral. Parenteral administration includes intracranial, intrathecal or intraventricular administration.

mivelsiran can be delivered in a manner to target a particular tissue, such as the CNS (e.g., neuronal, glial and/or vascular tissue of the brain).

### Carrier Compounds

Certain compositions of the present disclosure also incorporate carrier compounds in the formulation. As used herein, a “carrier compound” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with a carrier compound polyinosinic acid, dextran sulfate, polycytidic acid, or 4-acetamido-4′isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

### Carriers and Excipients

In contrast to a carrier compound, a “carrier,” a “pharmaceutical carrier,” a “pharmaceutically acceptable carrier,” “excipient,” or a “pharmaceutically acceptable excipient” is a pharmaceutically acceptable material, composition or vehicle, such as a liquid or solid filler, diluent, excipient, manufacturing aid (e.g., lubricant, talc magnesium, calcium or zinc stearate, or steric acid), solvent, suspending agent or any other pharmacologically inert vehicle for delivering one or more nucleic acids to an animal, or from one organ or portion of the body, to another organ or portion of the body. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred.

The data obtained from cell culture assays and animal studies can be used in formulating a range of dosage for use in humans. The dosage of compositions featured herein in the disclosure lies generally within a range of circulating concentrations that include the ED₅₀ with little or no toxicity. The dosage can vary within this range depending upon the dosage form employed and the route of administration utilized. For any compound used in the methods featured in the disclosure, the therapeutically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

In addition to their administration, as discussed above, the RNAi agents featured in the disclosure can be administered in combination with other known agents effective in treatment of pathological processes mediated by APP expression. In any event, the administering physician can adjust the amount and timing of RNAi agent administration on the basis of results observed using standard measures of efficacy known in the art or described herein.

### Kits

In certain aspects, the instant disclosure provides kits that include a suitable container containing a pharmaceutical formulation of mivelsiran. In certain embodiments the individual components of the pharmaceutical formulation may be provided in one container. Alternatively, it may be desirable to provide the components of the pharmaceutical formulation separately in two or more containers, e.g., one container for a siRNA compound preparation, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

### Methods for Inhibiting APP Expression

The present disclosure also provides methods of inhibiting expression of an APP gene in a cell. The methods include contacting a cell with an RNAi agent, e.g., double stranded RNAi agent, in an amount effective to inhibit expression of APP in the cell, thereby inhibiting expression of APP in the cell. In certain embodiments of the disclosure, APP is inhibited preferentially in CNS (e.g., brain) cells.

Contacting of a cell with a RNAi agent, e.g., a double stranded RNAi agent, may be done in vitro or in vivo. Contacting a cell in vivo with the RNAi agent includes contacting a cell or group of cells within a subject, e.g., a human subject, with the RNAi agent. Combinations of in vitro and in vivo methods of contacting a cell are also possible.

Contacting a cell may be direct or indirect, as discussed above. Furthermore, contacting a cell may be accomplished via a targeting ligand, including any ligand described herein or known in the art. In some embodiments, the targeting ligand is a carbohydrate moiety, e.g., a C16 ligand, or any other ligand that directs the RNAi agent to a site of interest.

The term “inhibiting,” as used herein, is used interchangeably with “reducing,” “silencing,” “downregulating,” “suppressing” and other similar terms, and includes any level of inhibition. In certain embodiments, a level of inhibition, e.g., for mivelsiran or a pharmaceutically acceptable salt thereof, can be assessed in cell culture conditions, e.g., wherein cells in cell culture are transfected via Lipofectamine™-mediated transfection at a concentration in the vicinity of a cell of 10 nM or less, 1 nM or less, etc. Knockdown of a given RNAi agent can be determined via comparison of pre-treated levels in cell culture versus post-treated levels in cell culture, optionally also comparing against cells treated in parallel with a scrambled or other form of control RNAi agent. Knockdown in cell culture of, e.g., at least 10% or more, at least 20% or more, etc. can thereby be identified as indicative of “inhibiting” and/or “reducing”, “downregulating” or “suppressing”, etc. having occurred. It is expressly contemplated that assessment of targeted mRNA and/or encoded protein levels (and therefore an extent of “inhibiting”, etc.) can also be assessed in in vivo systems for mivelsiran, under properly controlled conditions as described in the art.

The phrase “inhibiting expression of an APP,” as used herein, includes inhibition of expression of a human APP gene as well as variants or mutants of an APP gene that encode an APP protein. Thus, the APP gene may be a wild-type APP gene, a mutant APP gene, or a transgenic APP gene in the context of a genetically manipulated cell, group of cells, or organism.

“Inhibiting expression of an APP gene” includes any level of inhibition of an APP gene, e.g., at least partial suppression of the expression of an APP gene, such as an inhibition by at least about 20%. In certain embodiments, inhibition is by at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%.

The expression of an APP gene may be assessed based on the level of any variable associated with APP gene expression, e.g., APP mRNA level or APP protein level (including APP cleavage products). The expression of an APP may also be assessed indirectly based on the levels of APP-associated biomarkers as described herein.

Inhibition may be assessed by a decrease in an absolute or relative level of one or more of these variables compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In certain embodiments, surrogate markers can be used to detect inhibition of APP. For example, effective prevention or treatment of an APP-associated disorder, e.g., a CNS disorder such as EOAD or other disorder, as demonstrated by acceptable diagnostic and monitoring criteria with an agent to reduce APP expression can be understood to demonstrate a clinically relevant reduction in APP.

In some embodiments of the methods of the disclosure, expression of an APP gene is inhibited by at least 20%, a 25%, 30%, 35%, 40%, 45%, 50%, 55%, 60%, 65%, 70%, 75%, 80%, 85%, 90%, or 95%, or to below the level of detection of the assay. In some embodiments, the methods provided herein reduce soluble APPα and APPβ (sAPPα and sAPPβ) levels in a body fluid (e.g., CSF) by at least 20%, a 25%, 30%, 35%, 40%, 45%, 50%, 55%, 60%, 65%, 70%, 75%, 80%, 85%, 90%, or 95%, or to below the level of detection of the assay, e.g., by about 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 55-60%, 55-70%, 55-80%, 55-90%, 55-100%, 60-70%, 60-80%, 60-90%, 60-100%, 70-80%, 70-90%, 70-100%, 75-85%, or 75-100%. In specific embodiments, the methods provided herein reduce sAPPα levels in the CSF by at least 55%, or by about 55-75%, and reduce sAPPβ levels in the CSF by at least 65%, or by about 65-85% as compared to the baseline or a control subject, e.g., without administration of the RNAi agent provided herein. In some embodiments, the inhibitory effect of a single dose administration of the RNAi agent on the sAPPα and sAPPβ is long-lasting, e.g., persists for at least 3, 4, 5, or 6 months. For example, following a single-dose administration of the RNAi agent provided herein, sAPPα can be reduced by at least about 55-80% and sAPPβ can be reduced by at least about 70-85%, and these inhibitory effects can be sustained for at least 4 months after single dose administration.

In certain embodiments, the methods include a clinically relevant inhibition of expression of APP, e.g., as demonstrated by a clinically relevant outcome after treatment of a subject with an agent to reduce the expression of APP.

### Methods of Treating or Preventing APP-Associated Diseases

The present disclosure also provides methods of using mivelsiran and/or a composition containing mivelsiran to reduce and/or inhibit APP expression in a cell. The methods include contacting the cell with mivelsiran and maintaining the cell for a time sufficient to obtain degradation of the mRNA transcript of an APP gene, thereby inhibiting expression of the APP gene in the cell. Reduction in gene expression can be assessed by any methods known in the art. For example, a reduction in the expression of APP may be determined by determining the mRNA expression level of APP using methods routine to one of ordinary skill in the art, e.g., Northern blotting, qRT-PCR; by determining the protein level of APP using methods routine to one of ordinary skill in the art, such as Western blotting, immunological techniques. A reduction in the expression of APP may also be assessed indirectly by measuring a decrease in the levels of a soluble cleavage product of APP, e.g., a decrease in the level of soluble APPα, APPβ and/or a soluble Aβ peptide, optionally in a CSF sample of a subject.

In the methods of the disclosure the cell may be contacted in vitro or in vivo, i.e., the cell may be within a subject.

A cell suitable for treatment using the methods of the disclosure may be any cell that expresses an APP gene. A cell suitable for use in the methods of the disclosure may be a human cell, e.g., a human CNS cell.

APP expression is inhibited in the cell relative to a reference level or a control. A “reference level” and a “control level” can be used interchangeably herein. The inhibition of APP expression can be measured as compared to the APP expression in a control cell, which can be the cell prior to being contacted by the RNAi agent, or a reference level of APP expression in a reference cell or a population of reference cells being contacted with a vehicle or not being contacted with the RNAi agent. The inhibition can also be measured as compared to the predetermined threshold level as a reference level. One having ordinary skills in the art is able to select an appropriate control or reference. APP (e.g., sAPPα, sAPPβ) expression levels can be measured by standard methods for measuring mRNA or protein levels, including RT-PCR, PCR, Western blotting, and ELISA of a sample from the subject. For example, APP expression (sAPPα, sAPPβ) can be inhibited by at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100% relative to a control. In preferred embodiments, APP expression is inhibited by at least 20% relative to the control. Inhibition of APP (e.g., sAPPα, sAPPβ) expression in the cell can be sustained for a duration of time, such as at least 1 week, 2 weeks, 1 month, 2 months, 3 months, 4 months, 5 months, 6 months, 9 months, 10 months, 1 year, or more than 1 year following an administration of a single dose of the RNAi agent provided herein. In some embodiments, APP expression is inhibited by at least 30%, 40%, 50%, 55%, 60%, 70%, 80%, 90%, 95%, or more than 95% in the cell for at least 1 week, 2 weeks, 1 month, 2 months, 3 months, 4 months, 5 months, 6 months, 9 months, 10 months, 1 year, or more than 1 year, for example inhibited by at least 40% for at least 3 months, by at least 40% for at least 6 months, by at least 40% for at least 10 months, by at least 55% for at least 3 months, by at least 55% for at least 6 months, by at least 55% for at least 10 months, or any combination of the % decrease and duration set forth in Table 13, after being contacted by a single dose of the RNAi agent provided herein.

The in vivo methods of the disclosure may include administering to a subject a composition containing a RNAi agent, where the RNAi agent includes a nucleotide sequence that is complementary to at least a part of an RNA transcript of the APP gene of the mammal to be treated. When the organism to be treated is a mammal such as a human, the composition can be administered by any means known in the art including, but not limited to parenteral routes, including intracranial, e.g., intraventricular, intraparenchymal and intrathecal.

In some embodiments, the administration is via a pump. The pump may be an external pump or a surgically implanted pump. In certain embodiments, the pump is a implanted osmotic pump. In other embodiments, the pump is an infusion pump. In other embodiments, the pump is a surgically implanted pump that delivers the RNAi agent to the CNS.

In one aspect, the present disclosure also provides methods for inhibiting the expression of an APP gene in a mammal. The methods include administering to the mammal a composition comprising a dsRNA that targets an APP gene in a cell of the mammal and maintaining the mammal for a time sufficient to obtain degradation of the mRNA transcript of the APP gene, thereby inhibiting expression of the APP gene in the cell. Reduction in gene expression can be assessed by any methods known it the art and by methods, e.g., qRT-PCR, described herein. Reduction in protein production can be assessed by any methods known it the art and by methods, e.g., ELISA, described herein. In one embodiment, a CNS biopsy sample or a cerebrospinal fluid (CSF) sample serves as the tissue material for monitoring the reduction in APP gene and/or protein expression (or of a proxy therefore, as described herein or as known in the art).

The present disclosure further provides methods of treatment of a subject in need thereof. The treatment methods of the disclosure include administering mivelsiran or a pharmaceutically acceptable salt thereof to a subject, e.g., a subject that would benefit from a reduction and/or inhibition of APP expression, in a therapeutically effective amount of a RNAi agent targeting an APP gene or a pharmaceutical composition comprising a RNAi agent targeting an APP gene.

The present disclosure also provides methods of decreasing Aβ40 and/or Aβ42 levels in a subject. The methods include administering mivelsiran to a subject, e.g., a subject that would benefit from a reduction and/or inhibition of APP expression, in a therapeutically effective amount of a RNAi agent targeting an APP gene or a pharmaceutical composition comprising a RNAi agent targeting an APP gene.

In addition, the present disclosure provides methods of preventing, treating and/or inhibiting the progression of an APP-associated disease or disorder (e.g., AD, optionally EOAD) in a subject, such as the progression of an APP-associated disease or disorder to neurodegeneration, increased amyloid plaque formation and/or cognitive decline in a subject having an APP-associated disease or disorder or a subject at risk of developing an APP-associated disease or disorder. The methods include administering to the subject a therapeutically effective amount of any of the dsRNAs or the pharmaceutical composition provided herein, thereby preventing, treating and/or inhibiting the progression of an APP-associated disease or disorder in the subject.

mivelsiran may be administered as a “free RNAi agent.” A free RNAi agent is administered in the absence of a pharmaceutical composition. The naked RNAi agent may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the RNAi agent can be adjusted such that it is suitable for administering to a subject.

Alternatively, mivelsiran may be administered as a pharmaceutical composition, such as a dsRNA liposomal formulation.

Subjects that would benefit from a reduction and/or inhibition of APP gene expression are those having an APP-associated disorder. The term “APP-associated disease” includes a disease, disorder or condition that would benefit from a decrease in APP gene expression, replication, or protein activity. Non-limiting examples of APP-associated diseases include, for example, AD (including EOAD, sporadic and/or late onset AD, optionally with CAA).

The disclosure further provides methods for the use of a RNAi agent or a pharmaceutical composition thereof, e.g., for treating a subject that would benefit from reduction and/or inhibition of APP expression, e.g., a subject having an APP-associated disorder, in combination with other pharmaceuticals and/or other therapeutic methods, e.g., with known pharmaceuticals and/or known therapeutic methods, such as, for example, those which are currently employed for treating these disorders. For example, in certain embodiments, a RNAi agent targeting APP is administered in combination with, e.g., an agent useful in treating an APP-associated disorder as described elsewhere herein or as otherwise known in the art. For example, additional agents suitable for treating a subject that would benefit from reduction in APP expression, e.g., a subject having an APP-associated disorder, may include agents currently used to treat symptoms of AD. Non-limiting examples of such agents may include cholinesterase inhibitors (such as donepezil, rivastigmate, and galantamine), memantine, BACE1i, immunotherapies, and secretase inhibitors. The RNAi agent and additional therapeutic agents may be administered at the same time and/or in the same combination, e.g., intrathecally, or the additional therapeutic agent can be administered as part of a separate composition or at separate times and/or by another method known in the art or described herein.

In one embodiment, the method includes administering a composition featured herein such that expression of the target APP gene is decreased, such as for about 1, 2, 3, 4, 5, 6, 7, 8, 12, 16, 18, 24 hours, 28, 32, or about 36 hours. In one embodiment, expression of the target APP gene is decreased for an extended duration, e.g., at least about two, three, four days or more, e.g., about one week, two weeks, three weeks, or four weeks or longer.

Compositions and methods for inhibiting the expression of these genes using RNAi agents can be prepared and performed as described herein.

Administration of the dsRNA according to the methods of the disclosure may result in a reduction of the severity, signs, symptoms, and/or markers of such diseases or disorders in a subject with an APP-associated disorder. By “reduction” in this context is meant a statistically significant decrease in such level relative to a control. The reduction can be measured as compared to the severity, signs, symptoms, and/or markers in a control or reference subject, which can be the subject prior to being administered the dsRNA agent, or a reference subject or a population of reference subjects (e.g., an age- and sex-matched non-diseased population) being administered a placebo agent or not being administered the RNAi agent, such as a reference level for the severity, signs, symptoms, and/or markers in a healthy population. The reduction can also be measured as compared to the predetermined threshold level as a reference level. One having ordinary skills in the art is able to select an appropriate control or reference. Example signs, symptoms, and markers of APP-associated disorders are disclosed further in the present disclosure. Example markers include APPα, APPβ, and NfL. The reduction can be, for example, at least about 5%, 10%, 15%, 20%, 25%, 30%, 35%, 40%, 45%, 50%, 55%, 60%, 65%, 70%, 75%, 80%, 85%, 90%, 95%, or about 100% relative to a control. Reduction of the severity, signs, symptoms, and/or markers of the APP-associated disorder (e.g., EOAD) in the subject can be sustained for a duration of time, such as at least 1 week, 2 weeks, 1 month, 2 months, 3 months, 4 months, 5 months, 6 months, 9 months, 10 months, 1 year, or more than 1 year following an administration of a single dose of the RNAi agent provided herein. In some embodiments, the severity, signs, symptoms, and/or markers of the APP-associated disorder are reduced by at least 5%, 10%, 20%, 30%, 40%, 50%, 55%, 60%, 70%, 80%, 90%, 95%, or more than 95% in the subject for at least 1 week, 2 weeks, 1 month, 2 months, 3 months, 4 months, 5 months, 6 months, 9 months, 10 months, 1 year, or more than 1 year, for example, reduced by at least 40% for at least 3 months, by at least 40% for at least 6 months, by at least 40% for at least 10 months, by at least 55% for at least 3 months, by at least 55% for at least 6 months, by at least 55% for at least 10 months, or any combination of the % decrease and duration set forth in Table 13, following an administration of a single dose of the RNAi agent provided herein.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. For example, efficacy of treatment of an APP-associated disorder may be assessed, for example, by periodic monitoring of a subject's cognition and/or presence or amount of disease biomarkers in the body fluid (e.g., CSF, blood, plasma, serum).

In some embodiments, the effect of mivelsiran on CSF levels of disease biomarkers, including but not limited to Aβ40, Aβ42, neurofilament light chain protein (NfL), neurofilament heavy chain protein (NfH), apolipoprotein E (APOE), neurogranin, synaptosome associated protein 25 (SNAP25), S100 calcium binding protein B (S100B), t-Tau, and p-Tau (e.g., p-Tau181), is monitored. In addition, inflammatory biomarkers including but not limited to complement component 3 (C3), complement component 5 (C5), interleukin-6 (IL-6), interleukin-1 (IL-1), tumor necrosis factor (TNF), monocyte chemoattractant protein-1 (MCP-1), chitinase-3-like protein 1 (YKL-40), can be monitored. In some embodiments, plasma levels of Aβ40, Aβ42, NfL, t-Tau, and p-Tau species are also measured. Without wishing to be bound by theory, these biomarkers are associated with amyloidopathy, tauopathy, neuroinflammation, and neurodegeneration, collectively allowing for the characterization of underlying disease pathology.

Biomarkers of disease activity that can be assessed include Aβ40, Aβ42, NFL, NfH, APOE, neurogranin, SNAP25, S100B, t-Tau, and p-Tau (e.g., p-Tau181) species. As described elsewhere herein, Aβ40, Aβ42 are secretase-cleaved forms of APP and are commonly found as the predominant protein in amyloid beta plaques. Aβ40 and Aβ42 have long been described as associated with the development and progression of Alzheimer's disease (AD) in affected individuals and can be detected in the CSF. NfL and NfH are neurofilaments which are intermediate filaments of 10 nm that are highly expressed in neurons. Damage to neuronal axons causes release of NfL and NfH into the CSF and blood and are markers for neurological damage and can serve as markers to monitor disease activity (Gaetani L, et al. (2019) Journal of Neurology, Neurosurgery & Psychiatry 90:870-881). NfL and NfH can be detected in CSF as well as serum and plasma. APOE is a cholesterol carrier that contributes to injury repair and lipid transport and in the CNS is mainly produced by astrocytes. Polymorphic alleles of APOE are related to AD risk. For example, the ϵ4 allele of the APOE gene is associated with increased risk of AD. APOE can bind amyloid-β (Aβ) and regulate the metabolism, aggregation, and deposition of Aβ and thus contributes to AD risk (Kanekiyo et al. (2014) Neuron 81(4):740-754). Neurogranin is highly expressed in the brain and is a neuron-specific post-synaptic protein that regulates calcium-mediated signaling pathways that impact cognitive function. Increased levels of neurogranin in CSF and blood is associated with AD and cognitive decline (Liu, W. et al. (2020) Transl Psychiatry 10, 125). SNAP25 is mostly localized in nerve terminals and is involved in synaptic vesicular exocytosis. SNAP25 is a marker of synaptic damage and is increased in the CSF of AD patients (Zhang, H. et al. (2018) Alz Res Therapy 10, 80). S100B is calcium-binding protein mainly expressed and secreted by astrocytes in the CNS and is a marker of astroglial damage. S100B is involved in processing of amyloid precursor protein, regulation of Aβ peptide levels and Tau phosphorylation. S100B can act as a pro-inflammatory molecule and have toxic effects on neurons, depending on its concentration. Elevated levels of S100B have been detected in the CSF and blood of AD patients and high levels of S100B may be involved in APP cleavage processes (Cristóvão J S and Gomes C M. (2019) Front Neurosci. 13:463). The tau protein is predominantly expressed in neurons and is a microtubule-associated protein that functions in the stabilization of microtubules. The neurofibrillary tangles of AD comprise accumulations of the modified tau protein. In AD, tau becomes hyperphosphorylated (p-Tau) leading to microtubule disassembly and free tau molecule aggregation. T-Tau and p-Tau (e.g., p-Tau181) in the CSF and blood are both markers of neurodegeneration and AD (Medeiros R, et al. (2011) CNS Neurosci Ther. 17(5):514-24).

The administration of mivelsiran to the subject can produce a reduction in the levels in the CSF of disease biomarkers, such as Aβ40, Aβ42, NfL, NfH, APOE, neurogranin, SNAP25, S100B, t-Tau, and p-Tau (e.g., p-Tau181). In some embodiments, the subject shows about 10%, 20%, 30%, 40%, 50%, 60%, 70%, 80%, or 90% decrease, or at least about 10%, 20%, 30%, 40%, 50%, 60%, 70%, 80%, or 90% reduction in the levels in the CSF of disease biomarkers, such as Aβ40, Aβ42, NfL, NfH, APOE, neurogranin, SNAP25, S100B, t-Tau, and p-Tau following administration of mivelsiran or the equivalent amount of a pharmaceutically acceptable salt thereof (e.g., sodium salt). By “reduction” in this context is meant a statistically significant decrease in such level relative to a control or a reference level. A “control level” and a “reference level” can be used interchangeably herein. The reduction can be measured as compared to the biomarker level in a control subject, which can be the subject prior to being administered the RNAi agent, or a reference subject or a population of reference subjects being administered a placebo agent or not being administered the RNAi agent, such as a reference biomarker level in a healthy population. The reduction can also be measured as compared to the predetermined threshold level as a reference level. One having ordinary skill in the art is able to select an appropriate control or reference. In specific embodiments, the subject shows at least about 30% decrease in the level of Aβ42 and/or at least about 50% decrease in the level of Aβ40 in the CSF following administration of mivelsiran or the equivalent amount of a pharmaceutically acceptable salt thereof (e.g., sodium salt). In some embodiments, the subject shows at least about 40% decrease in the level of Aβ42 and/or at least about 60% decrease in the level of Aβ40 in the CSF following administration of mivelsiran or the equivalent amount of a pharmaceutically acceptable salt thereof (e.g., sodium salt). CSF levels of a disease biomarker can be measured by standard methods for measuring mRNA or protein levels of a sample from the subject, including RT-PCR, qPCR, Northern blotting, Western blotting, ELISA, or other immunologic method.

In addition, inflammatory biomarkers in the body fluid (e.g., CSF, blood, plasma, serum) can be monitored. Non-limiting examples of inflammatory markers include, complement component 3 (C3), complement component 5 (C5), interleukin-6 (IL-6), interleukin-1 (IL-1), tumor necrosis factor (TNF), monocyte chemoattractant protein-1 (MCP-1), and chitinase-3-like protein 1 (YKL-40 or CH13L1). C3 and C5 are components of the complement system, found primarily in the blood, which is part of the innate immune system. Elevated C3 and/or C5 is a marker of inflammation. IL-6 is a soluble inflammatory molecule and has roles in both innate and adaptive immune responses and may contribute to chronic inflammation. As such, elevated IL-6 levels in the blood are a marker of inflammation (Tanaka T, et al. (2014) Cold Spring Harb Perspect Biol. 6(10):a016295). IL-1 is a family of cytokines, including IL-1α and IL-1β, which are associated with acute and chronic inflammation. While IL-1 has been linked to damaging inflammation, it plays a key role in innate immunity (Dinarello CA. (2018) Immunol Rev. 2018 281(1):8-27). TNF is considered a central inflammatory cytokine and has been the target of treatments for chronic inflammatory diseases. In addition to driving inflammatory responses, TNF also induces cell death which indirectly induces inflammatory responses (van Loo, G. and Bertrand, M. J. M. (2023) Nat Rev Immunol 23, 289-303). As such, elevated TNF can be used as a marker of inflammation. MCP-1 is a chemokine that regulates monocyte/macrophage, T lymphocyte, and natural killer cell migration and infiltration to sites of infection or tissue injury and is therefore an indicator of inflammation (Deshmane S L, et al. (2009) J Interferon Cytokine Res. 29(6):313-26). YKL-40 or CH13L1 is a glycoprotein produced by inflammatory cells and has been shown to be elevated in brain and CSF in neurodegenerative diseases. As such, YKL-40 can be used as a marker of neuroinflammation (Llorens, F. et al. (2017) Mol Neurodegeneration 12, 83).

The administration of mivelsiran to the subject can reduce the levels of inflammatory biomarkers, such as C3, C5, IL-6, IL-1, TNF, MCP-1, YKL-40, and CH13L1, in a body fluid sample. In some embodiments, the subject shows about 10%, 20%, 30%, 40%, 50%, 60%, 70%, 80%, or 90% decrease, or at least about 10%, 20%, 30%, 40%, 50%, 60%, 70%, 80%, or 90% decrease in the levels in a body fluid sample of inflammatory biomarkers, such as C3, C5, IL-6, IL-1, TNF, MCP-1, YKL-40, and CH13L1 following administration of mivelsiran or the equivalent amount of a pharmaceutically acceptable salt thereof (e.g., sodium salt). By “reduction” in this context is meant a statistically significant decrease in such level relative to a control or a reference level. A “control level” and a “reference level” can be used interchangeably herein. The reduction can be measured as compared to the biomarker level in a control subject, which can be the subject prior to being administered the RNAi agent, or a reference subject or a population of reference subjects being administered a placebo agent or not being administered the RNAi agent, such as a reference biomarker level in a healthy population. The reduction can also be measured as compared to the predetermined threshold level as a reference level. One having ordinary skill in the art is able to select an appropriate control or reference. Levels of a disease biomarker can be measured by standard methods for measuring mRNA or protein levels of a sample from the subject, including RT-PCR, qPCR, Northern blotting, Western blotting, ELISA, or other immunologic method.

Comparisons of the later readings with the initial readings provide a physician an indication of whether the treatment is effective. It is well within the ability of one skilled in the art to monitor efficacy of treatment or prevention by measuring any one of such parameters, or any combination of parameters. In connection with the administration of a RNAi agent targeting APP or pharmaceutical composition thereof, “effective against” an APP-associated disorder indicates that administration in a clinically appropriate manner results in a beneficial effect for at least a statistically significant fraction of patients, such as an improvement of symptoms, a cure, a reduction in disease, extension of life, improvement in quality of life, or other effect generally recognized as positive by medical doctors familiar with treating APP-associated disorders and the related causes.

A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given RNAi agent drug or formulation of that drug can also be judged using an experimental animal model for the given disease as known in the art. When using an experimental animal model, efficacy of treatment is evidenced when a statistically significant reduction in a marker or symptom is observed.

Alternatively, the efficacy can be measured by a reduction in the severity of disease as determined by one skilled in the art of diagnosis based on a clinically accepted disease severity grading scale, as but one example mental ability tests for dementia. Any positive change resulting in e.g., lessening of severity of disease measured using the appropriate scale, represents adequate treatment using a RNAi agent or RNAi agent formulation as described herein.

Subjects can be administered a therapeutic amount of dsRNA, such as about 0.01 mg/kg to about 200 mg/kg.

The RNAi agent can be administered intrathecally over a period of time, on a regular basis. In certain embodiments, after an initial treatment regimen, the treatments can be administered on a less frequent basis. Administration of the RNAi agent can reduce APP levels, e.g., in a cell, tissue, blood, CSF sample or other compartment of the subject. By “reduction” in this context is meant a statistically significant decrease in such level relative to a control or a reference level. A “control level” and a “reference level” can be used interchangeably herein. The reduction can be measured as compared to the APP level in a control subject, which can be the subject prior to being administered the RNAi agent, or a reference subject or a population of reference subjects being administered a placebo agent or not being administered the RNAi agent, such as a reference APP level in a healthy population. The reduction can also be measured as compared to the predetermined threshold level as a reference level. One having ordinary skill in the art is able to select an appropriate control or reference. APP (e.g., sAPPα, sAPPβ) levels in a sample can be measured by standard methods for measuring mRNA or protein levels, including RT-PCR, PCR, Western blotting, and ELISA of a sample from the subject. For example, APP (sAPPα, sAPPβ) levels can be reduced by at least about 5%, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 39, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or at least about 99% or more. In a preferred embodiment, administration of the RNAi agent can reduce APP levels, e.g., in a cell, tissue, blood, CSF sample or other compartment of the subject by at least 20%. Reduction of APP (e.g., sAPPα, sAPPβ) levels in the subject (e.g., in a cell, tissue, blood, CSF sample or other compartment) can be sustained for a duration of time, such as at least 1 week, 2 weeks, 1 month, 2 months, 3 months, 4 months, 5 months, 6 months, 9 months, 10 months, 1 year, or more than 1 year following an administration of a single dose of the RNAi agent provided herein. In some embodiments, reduction of APP levels in the subject is inhibited by at least 30%, 40%, 50%, 55%, 60%, 70%, 80%, 90%, 95%, or more than 95% for at least 1 week, 2 weeks, 1 month, 2 months, 3 months, 4 months, 5 months, 6 months, 9 months, 10 months, 1 year, or more than 1 year, for example by at least 40% for at least 3 months, by at least 40% for at least 6 months, by at least 40% for at least 10 months, by at least 55% for at least 3 months, by at least 55% for at least 6 months, by at least 55% for at least 10 months, or any combination of the % decrease and duration set forth in Table 13, following an administration of a single dose of the RNAi agent provided herein.

Before administration of a full dose of the RNAi agent, patients can be administered a smaller dose, such as a 5% infusion reaction, and monitored for adverse effects, such as an allergic reaction. In another example, the patient can be monitored for unwanted immunostimulatory effects, such as increased cytokine (e.g., TNF-alpha or INF-alpha) levels.

One or more injections may be used to deliver the desired, e.g., monthly dose of RNAi agent to a subject. The injections may be repeated over a period of time. The administration may be repeated on a regular basis. In certain embodiments, after an initial treatment regimen, the treatments can be administered on a less frequent basis. A repeat-dose regimen may include administration of a therapeutic amount of RNAi agent on a regular basis, such as monthly or extending to once a year or once every 2, 3, 4 and/or 5 years. In certain embodiments, the RNAi agent is administered about once per month to about once per quarter (i.e., about once every three months).

Unless otherwise defined, all technical and scientific terms used herein have the same meaning as commonly understood by one of ordinary skill in the art to which this invention belongs. Although methods and materials similar or equivalent to those described herein can be used in the practice or testing of the RNAi agents and methods featured in the invention, suitable methods and materials are described below. In addition, the materials, methods, and examples are illustrative only and not intended to be limiting.

### EXAMPLES

### Example 1. Treating Subjects Having or at Risk of Developing Early Onset Alzheimer's Disease with a Nucleic Acid Inhibitor of APP

Alzheimer's disease is the most common neurodegenerative disease and the most common form of dementia worldwide. According to the World Health Organization (WHO), approximately 50 million people worldwide are affected by dementia, and AD is thought to be the underlying cause in 60% to 70% of dementia, amounting to 30 to 35 million affected worldwide (World Health Organization 2020 Dementia).

Alzheimer's disease is characterized by progressive memory loss and cognitive decline, with neuropathological accumulation of amyloid plaques, neurofibrillary tangles, and neuroinflammation. Although the disease pattern is uniform, the etiologies are heterogeneous. The exact pathomechanisms of AD remain controversial, but genetic studies over the last 30 years have provided insight into the role of APP in the disease. It is well established that genetic alterations in APP expression and proteolysis results in early-onset forms of AD. Amyloid precursor protein locus duplications and trisomy 21 (Down's syndrome) result in quantitative increases in APP expression and are associated with early-onset forms of AD. Mutations in the genes for APP, presenilin 1 (PSEN1), and presenilin 2 (PSEN2) have been shown to alter the proteolysis of APP, resulting in increased Aβ accumulation, leading to early-onset forms of AD. Lastly, rare mutations in APP result in a reduction of the formation of Aβ in Icelandic individuals and have been shown to be protective from AD and associated with increased survival. Together, this evidence suggests that reducing the production of APP and thereby reducing downstream APP cleavage products such as Aβ may be an effective therapeutic strategy for AD.

Early-onset Alzheimer's disease refers to a subgroup with AD symptom onset prior to the age of 65 years, representing approximately 4% to 6% of all AD making EOAD the most common early-onset neurodegenerative dementia. [Mendez 2017] About 14% of patients with EOAD have an autosomal dominant history, but only 1.6% of the EOAD population has an identified monogenic cause. These patients represent a rare subtype called Autosomal Dominant Alzheimer's disease (ADAD), which is caused by a mutation in 1 of the 3 identified genes directly involved in the production of Aβ peptides: APP, PSEN1, or PSEN2. [Bateman 2011; Muller 2013; Nussbaum and Ellis 2003] Although rare, the ADAD subgroup provides important mechanistic support that EOAD originates from abnormal APP metabolism. Clinical presentation of ADAD is similar to sporadic AD, but disease onset is earlier and progression is rapid, leading to fatality in approximately 6 to 9 years after symptoms onset. [Bateman 2011]

Treatments currently approved for AD-type dementia include acetylcholinesterase inhibitors and N-methyl-d-aspartate receptor antagonists which only aim at treating symptoms of the disease. [Cummings 2019] Multiple therapeutic monoclonal anti-Aβ antibodies have been tested in AD. Despite the clearance of extracellular amyloid plaques in the brain, demonstration of clinical efficacy with anti-Aβ antibodies has been challenging, although Aduhelm® (aducanumab) recently received accelerated approval in the USA for the treatment of AD, based on reduction in Aβ plaques observed in patients with mild cognitive impairment or mild dementia treated with aducanumab. Consequently, there remains a significant unmet need for treatments that can slow, halt, or even potentially reverse the pathogenetic cascade common to EOAD.

A need therefore exists for agents that can treat or prevent APP-associated diseases or disorders in an affected individual.

Accordingly, the present invention provides methods for treating subjects having or at risk of developing a EOAD or disorder that would benefit from reduction in APP using nucleic acid inhibitors, e.g., double stranded ribonucleic acid (dsRNA) agents or single stranded antisense polynucleotide agents targeting APP, as described herein.

TABLE 1
Abbreviations of nucleotide monomers used in nucleic acid sequence
representation.
It will be understood that these monomers, when present in an oligonucleotide, are mutually
linked by 3′->5′-phosphodiester bonds, and it is understood that when the nucleotide contains a 2′-
fluoro modification, then the fluoro replaces the hydroxy at that position of the parent nucleotide (i.e.,
it is a 2′-deoxy-2′-fluoronucleotide). It is understood that the following abbreviations omit the “3-
phosphate” when located at the 3′-terminal position of an oligonucleotide (i.e., the 3′-terminal
position is a 3′-OH).

| Abbreviation | Nucleotide(s) |
|---|---|
| A | Adenosine-3′-phosphate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| a | 2′-O-methyladenosine-3′-phosphate |
| as | 2′-O-methyladenosine-3′-phosphorothioate |
| C | 2′-O-methylcytidine-3′-phosphate |
| CS | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| S | phosphorothioate linkage |
| VP | Vinyl-phosphonate (e.g., 5′-(E)-vinyl phosphonate) |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| (Agn) | Adenosine-glycol nucleic acid (GNA) S-isomer |
| (Cgn) | Cytidine-glycol nucleic acid (GNA) S-isomer |
| (Ggn) | Guanosine-glycol nucleic acid (GNA) S-isomer |
| (Tgn) | Thymidine-glycol nucleic acid (GNA) S-Isomer 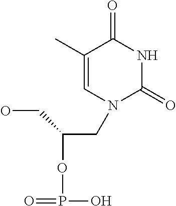 |
| (Ahd) | 2′-O-hexadecyl-adenosine-3′-phosphate |
| (Chd) | 2′-O-hexadecyl-cytidine-3′-phosphate 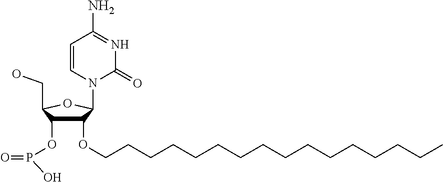 |
| (Ghd) | 2′-O-hexadecyl-guanosine-3′-phosphate |
| (Uhd) | 2′-O-hexadecyl-uridine-3′-phosphate |
| VPus | 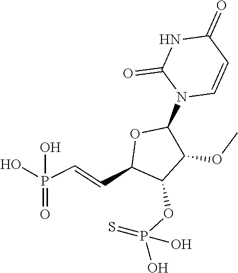 [SMILES: CO[C@@H]1[C@H](OP(O)(O)=S)[C@@H](/C=C/P(=O)(O)O)O[C@H]1n1ccc(=O)[nH]c1=O] 5′-(E-(dihydroxy)phosphinylmethylidene)-5′-deoxy-2′-O-methyluridine 3′- phosphorothioate |

TABLE 2
Mivelsiran, modified and unmodified sequences

| | | | SEQ | | SEQ |
|---|---|---|---|---|---|
| | | Oligonucleotide | ID | Transcript | ID |
| Duplex | Strand | Sequence | NO: | Sequence | NO: |
| AD- | Sense | gsgscua(Chd)gad | 30 | GGCUACGAAA | 32 |
| 961583 | (5′ to 3′) | AadAuccaaccusas | | AUCCAACCUA | |
| | | a | | A | |
| | Antisense | VPusUfsaggu(Tgn) | 31 | UUAGGUTGGA | 33 |
| | (5′ to 3′) | ggaudTuUfcdGuag | | UTUUCGUAGC | |
| | | ccsgsu | | CGU | |

### Example 2. A Randomized, Double-Blind, Placebo-Controlled Single Ascending Dose and Open-Label Multi-Dose Study to Evaluate the Safety, Tolerability, Pharmacokinetics and Pharmacodynamics of Intrathecally Administered Mivelsiran in Adult Patients with Early-Onset Alzheimer's Disease (EOAD)

### Clinical Trial Protocols

### Summary of Study Design

The study is a two-part randomized, placebo-controlled, double blind, multicenter, multinational, human Phase 1 study to evaluate the safety, tolerability, PK, and PD of intrathecally administered mivelsiran in adult patients with EOAD. Part A is a randomized, double-blind, placebo-controlled single-ascending dose (SAD) period and Part B is a multi-dose open-label period including patients previously enrolled in Part A or their replacement who are allowed to enter Part B.

This study is conducted at approximately 10 clinical study centers, with enrollment of up to 60 patients in Part A and Part B.

A single dose of study drug (mivelsiran or placebo) was administered to each patient in Part A. In Part B, up to 3 mivelsiran dosing regimens is evaluated (Table 5).

Mivelsiran was supplied in the form of a salt, i.e., mivelsiran sodium as described in FIG. 1, in an aqueous formulation suitable for intrathecal administration. Dosages in the Examples are quoted based on free acid.

### Part A—Single Dose Period

Four ascending dose cohorts (1, 2, 3, and 4) are enrolled sequentially as follows:
6 patients (4 mivelsiran and 2 placebo) are enrolled in each of Cohorts 1 and 2.
8 patients (6 mivelsiran and 2 placebo) are enrolled in each of Cohorts 3 and 4

Each patient received a single dose of study drug followed by a 6-month Evaluation Period and an up to 6-month Follow-up Period, if needed, to achieve Drug Washout.

The decision to proceed to dosing the next cohort and the actual dose to be administered were determined by the safety review committee (SRC) based on postdose safety and tolerability data through at least Month 1 in addition to Repeatable Battery for the Assessment of Neuropsychological Status (RBANS) score, CBC with differential, CSF sAPPα and sAPPβ levels, cumulative safety data, and all available pharmacokinetics (PK) and pharmacodynamics (PD) data.

Up to 4 optional cohorts are enrolled and dosed according to the same eligibility criteria as the planned cohorts to better define dose response and/or safety and tolerability. The decision to enroll an optional cohort is made by the SRC based on safety and tolerability data if further elucidation of dose response and/or safety and tolerability is considered necessary. It was initially determined that the maximum administered should not exceed 1200 mg. The optional cohorts contain 8 patients each (6 mivelsiran and 2 placebo).

After informed consent is obtained, patients were screened between Day −60 and Day −2. Eligible patients were admitted to the clinical study center on Day −2 to Day −1 to determine continued eligibility and for predose assessments (clinical, blood, and neuroimaging evaluations). Patients in each cohort were randomized to receive a single dose of study drug (mivelsiran or placebo) prior to administration on Day 1. Patients were discharged from the clinical study center on Day 2.

Assessments during the initial admission period included neurological, electrophysiological, and physical examination, vital signs, electrocardiograms (ECGs), blood and CSF sampling, and clinical laboratory analyses. Full standard neurological assessment (including non-dilated fundi) was performed approximately 3 hours postdose and prior to discharge from the study center.

Patients returned to the clinical study center on an outpatient basis for safety, tolerability, PK, and PD monitoring at specified visits through the Evaluation and the Follow-up Periods. Patients were determined to have completed Part A at or after Month 6 when sAPPα and sAPPβ levels have returned to ≥75% of the patient's Day 1 sAPP and sAPPβ level for 2 consecutive visits (Drug Washout) or at Month 12, whichever was earlier. Therefore, the Follow-up Period after Month 6 was only applicable to patients who do not have Drug Washout at Month 6. If a patient discontinues prematurely from the study during the 6-month Evaluation Period, Early Termination (ET) assessments was performed. The patient was also encouraged to return for follow-up visits until Drug Washout, or at 6 months after the ET, whichever was earlier.

### Part B—Multi-Dose Period

Patients in Part B are limited to those who participated in Part A or their replacements.

In Part B, up to 3 mivelsiran dosing regimens of repeat doses are evaluated over a 12-month Dosing Period, followed by a 6-month Evaluation/Follow-up Period and an up to 6-month Extended Follow-up Period. Dosing in Part B may begin when the Safety Review Committee (SRC) recommends the dosing regimen for the first cohort based on review of cumulative safety data and available PD data from at least three Part A cohorts through at least the Month 3 visit. Table 3 summarizes the features and limits of the Part B dosing regimens.

TABLE 3
Characteristics of Part B Dosing Regimens

| Features | Limits |
|---|---|
| 1. The term “dosing regimen” includes: | I. Doses administered in Part B does not exceed doses |
| (1) the dose level administered, (2) the | already evaluated and considered to have an acceptable |
| dosing frequency. | safety profile in Part A. |
| 2. Dosing regimens are determined in | II. The maximum dose administered does not exceed |
| accordance with safety, tolerability, and | 180 mg and cumulative annual dose does not exceed |
| available pharmacodynamics data. | 360 mg; or the maximum dose administered does not |
| | exceed 225 mg and cumulative annual dose does not |
| | exceed 450 mg. |
| | III. Doses will not be administered more frequently than |
| | once every 6 months. |
| | IV. Duration: Dosing does not extend beyond Month 12 |

Dosing in Part B may begin when the SRC recommends the dosing regimen for the first cohort based on review of cumulative safety data and available PD data from at least 3 Part A cohorts through at least the Month 3 visit. The selected dose needs to show an acceptable safety profile in Part A, should have achieved at least 25% reductions in CSF sAPPα and sAPPβ levels in ≥3 patients on active treatment, in a given Part A cohort, and should not exceed 180 mg and cumulative annual dose should not exceed 360 mg, or should not exceed 225 mg and cumulative annual dose should not exceed 450 mg. Doses should not be administered more frequently than once every 3 months (Table 4). Part B may start while the follow-up and/or dosing of certain Part A cohort(s) is ongoing.

The dose(s) does not exceed the highest dose that has been demonstrated to have an acceptable safety profile in Part A, and may include intermediate (lower) dose levels per SRC recommendation. An intermediate (lower) dose that has not been studied in Part A may be selected by the SRC based on PD modeling considerations for the proposed dosing regimen, and should be expected to achieve at least 25% reductions in CSF sAPPα and sAPPβ levels during the multi-dose regimen.

Individual patients from Part A become eligible for enrollment in Part B upon completion of Part A and are consented separately for Part B. For patients who have completed Part A prior to Part B dose selection, no additional visits are planned during the interim and patients are contacted to return to the study center upon initiation of Part B.

Patients have predose assessments (clinical, blood, ECGs, and neuroimaging evaluations) on Day 1 at the clinical study center, followed by the IT procedure which includes predose CSF collection and IT bolus injection of mivelsiran. Patients are observed for 24 hours and discharged from the clinical study center on Day 2.

### Scientific Rationale for Study Design

This study is a randomized, double-blind, placebo-controlled, multi-center Phase 1 SAD and open-label multi-dose study designed to evaluate the safety, tolerability, PK, and PD of mivelsiran, administered by IT injection in adult patients with EOAD.

The primary objective of the study is to evaluate the safety and tolerability of SADs and multiple doses of mivelsiran in patients with EOAD. A 6-month postdose Evaluation Period is planned following completion of single dosing in Part A, and a 6-month postdose Evaluation/Follow-up Period is planned after multiple dosing in Part B. The Part A Follow-up period is only applicable to patients who do not have Drug Washout at Month 6. The necessity and duration of the Part B Extended Follow-up period is further informed by safety data, PD durability, and PK/PD modeling, and is a maximum of 6 months.

Secondary objectives include the characterization of PK and PD effects of mivelsiran. The PD effects of mivelsiran are determined by the change from baseline in sAPPα and sAPPβ proteins in the CSF. These 2 proteins are the upstream, proximate substrates of Aβ40/42 that are detectable in the CSF with current analytical assays. Thus, sAPPα and sAPPβ can serve as surrogates for monitoring the reduction of APP mRNA, the direct target of mivelsiran.

The exploratory objectives are to evaluate the effect of mivelsiran on different neuroimaging markers, plasma biomarkers, and CSF biomarkers which are associated with amyloidopathy, tauopathy, neuroinflammation, and neurodegeneration, together with the effect on clinical progression using several cognitive, functional, and neuropsychiatric assessments.

A placebo-control group is being used as the primary comparator for the safety and tolerability of mivelsiran during the Part A double-blind period. The placebo control serves as a control for mivelsiran-related adverse events. In addition, since the CSF volume fluctuation associated with IT injection can cause alterations in CSF protein biomarkers, the placebo control also serves as a critical procedural control for PD and exploratory CSF biomarker assessments during Part A.

In Part B, all patients receive the active drug and there is no placebo control. This balances the judicious use of rare patients while still enabling the multi-dose safety and PK/PD learnings.

Rationale for Including Patients with Early-Onset Alzheimer's Disease

This first-in-human study for mivelsiran is conducted in early symptomatic patients with EOAD. At early stages of the disease, EOAD patients are active and independent in most areas of functioning, and are also anticipated to be capable of providing informed consent. It is necessary to conduct this study in patients instead of healthy volunteers for 2 reasons. First, given the abnormal APP metabolism in the patient population (abnormal balance between amyloid production and clearance), safety findings, and PK/PD characteristics are better understood if studied directly in patients with EOAD. Second, since mivelsiran is administered by IT injection, further consideration of the balance between benefit and risk justifies the investigation solely in a patient population.

The initial clinical strategy focuses on patients with EOAD where increased amyloid production outpaces natural clearance mechanisms, leading to earlier amyloid accumulation and symptom onset prior to 65 years old. This is supported by human genetic studies in which variants that overproduce Aβ result in earlier disease onset (Fleisher et al. (2015) JAMA Neurol., 72(3):316-324) whereas an allele that reduces Aβ production by approximately 40% is protective against AD (Jonsson et al. (2012) Nature, 488(7409):96-99). As compared to amyloid-targeting immunotherapies which increase Aβ clearance, mivelsiran targets APP mRNA and reduces the production of Aβ. This mechanism of action favors initial study in EOAD where increased Aβ production is thought to be the primary contributor to early amyloid deposition.

The early symptomatic EOAD population is anticipated to have more homogeneous disease symptoms and biomarker levels than patients with late-onset AD, allowing for improved characterization of safety, tolerability, and PK/PD after study drug treatment. Although exploratory, the effect of mivelsiran on measures of disease activity is likely more detectable in a homogeneous disease population. Patients with EOAD are included in this study early in their disease stage (corresponding to mild cognitive impairment [MCI] and mild dementia). For the purposes of this study, early symptoms in EOAD patients are defined clinically (Clinical Dementia Rating [CDR] of 0.5 or 1.0 and Mini Mental State Examination [MMSE]>20).

AD-type dementia is confirmed by AD biomarkers (pathology confirmed by CSF biomarkers or amyloid plaque detected by positron emission tomography [PET]). AD patients are believed to show better biomarker responses to treatment earlier in the disease as compared to more advanced disease stages with irreversible neuronal loss.

### Method of Assigning Patients to Treatment Groups

In Part A, patients were randomized 4:2 or 6:2 to the mivelsiran or placebo arm using the Interactive Response Technology (IRT).

In Part B, cohort assignment (up to 3 different dose regimens) occur when dosing regimen(s) is (are) selected by the SRC. The SRC assigns patients from a particular Part A cohort to an approved Part B dosing regimen to include a minimum of 6 patients for each dosing regimen.

The cohort size in Part B is determined based on the number of dosing regimens to be evaluated. The SRC sequentially assigns patients based on the order of study enrollment in Part A (i.e., first-in, first-out). To balance Part B cohort sizes, the SRC can transition patients from the same Part A cohort to different Part B dosing regimens, if necessary.

In Part A and Part B, each patient may participate in only 1 cohort.

### Selection and Removal of Patients

### Inclusion Criteria for Patients in Part A and Replacement Patients in Parts A and B

Patients are eligible to be included in the study if all the following criteria apply:

### Age and Sex

1. Male or female, aged 18 years or older at the time of informed consent

### Patient and Disease Characteristics

2. Individuals with mild cognitive impairment or mild dementia due to EOAD, where disease onset occurred at age <65 years, and AD diagnosis confirmed by CSF biomarkers or positive PET amyloid imaging obtained during Screening or with historical values.
3. CDR global score 0.5 or 1.0 and Mini Mental State Examination (MMSE)>20
4. Able and willing to meet all study requirements in the opinion of the Investigator, including travel to study center, procedures, measurements and visits, including:
   a. Adequately supportive psychosocial circumstances
   b. Able to undergo Magnetic Resonance Imaging (MRI) scans and able to tolerate them
   c. Body Mass Index (BMI) ≥18 and ≤34 kg/m² at Screening visit
   d. Able to tolerate LP
   e. Able to undergo PET

### Informed Consent

5. Patient is able to understand and is willing and able to comply with the study requirements and to provide written informed consent at the study onset. If the patient is unable to provide informed consent any time after study onset, legal guardian(s) is (are) willing and able to comply with the study requirements and to provide written informed consent. Legal guardians are only to be used where applicable under local regulations. Ongoing participation in the trial is contingent upon the patient maintaining mental capacity to provide informed consent. During the study, a patient's mental capacity is regularly assessed by the Investigator and the entire team who interact with the patient according to sites established process. This comprehensive evaluation may include assessments already performed during the study and incorporates the patient's general understanding of the study and its procedures. If the Investigator determines a potential loss in mental capacity, the patient's participation in the study is terminated if local regulations do not allow consent by a legal guardian. Otherwise, the patient is assigned a legal guardian as allowed per local law.
   Inclusion Criteria for Patients Who Transition from Part A to Part B

For patients who transition from Part A to Part B, their eligibility is reconfirmed on Day 1 using the following criteria:

### Patient Characteristics

1. Able and willing to meet all study requirements in the opinion of the Investigator, including travel to study center, procedures, measurements and visits, including:
   a. Adequately supportive psychosocial circumstances.
   b. Able to undergo Magnetic Resonance Imaging (MRI) scans and able to tolerate them
   c. Able to tolerate LP
   d. Able to undergo PET

### Informed Consent

2. Patient is able to understand and is willing and able to comply with the study requirements and to provide written informed consent at the study onset. If the patient is unable to provide informed consent any time after study onset, legal guardian(s) is (are) willing and able to comply with the study requirements and to provide written informed consent. Legal guardians are only to be used where applicable under local regulations. Ongoing participation in the trial is contingent upon the patient maintaining mental capacity to provide informed consent. During the study, a patient's mental capacity is regularly assessed by the Investigator and the entire team who interact with the patient according to sites established process. This comprehensive evaluation may include assessments already performed during the study and incorporates the patient's general understanding of the study and its procedures. If the Investigator determines a potential loss in mental capacity, the patient's participation in the study is terminated if local regulations do not allow consent by a legal guardian. Otherwise, the patient is assigned a legal guardian as allowed per local law.

### Exclusion Criteria for Patients in Part A and Replacement Patients in Parts A and B

Patients are excluded from the study if any of the following criteria apply:

### Disease-Specific Conditions

1. Non-Alzheimer's disease dementia

### Laboratory Assessments

2. Has any of the following laboratory parameter assessments at Screening:
   a. Alanine aminotransferase (ALT) or aspartate aminotransferase (AST)>2× upper limit of normal (ULN)
   b. Total bilirubin >1.5×ULN. Patients with elevated total bilirubin that is secondary to documented Gilbert's syndrome are eligible if the total bilirubin is <2×ULN
   c. International normalized ratio (INR)>1.4
   d. Platelet count <100,000/microliter (μL)
   e. Absolute neutrophil count <lower limit of normal (LLN) cells/μL or absolute lymphocyte count <LLN cells/μL
   f. Estimated glomerular filtration rate (eGFR)<45 mL/min/1.73 m² at Screening (calculated with Modification of Diet in Renal Disease [MDRD] formula)
3. Clinically significant ECG abnormalities at Screening, in the opinion of the Investigator, or a Friderica-corrected QT interval (QTcF)>450 msec at Screening
4. Has systolic blood pressure >150 mmHg and/or a diastolic blood pressure >90 mmHg after 10 minutes of rest at screening
5. Has known active human immunodeficiency virus (HIV) infection; or evidence of current or chronic hepatitis C virus (HCV) or hepatitis B virus (HBV) infection
6. Has active severe acute respiratory syndrome coronavirus 2 (SARS-CoV-2) infection

### Prior/Concomitant Therapy

7. Treatment with another investigational drug, biological agent, or device within 6 months of Screening, or 5 half-lives of investigational agent, whichever is longer. Any agent that has received health agency authorization (including for emergency use) by local or regional regulatory authorities is not considered investigational.
8. Use of the following medications is prohibited unless the dose has been stable for at least 12 weeks prior to Screening and the dose regimen is not anticipated to change during the study: antidepressants, antipsychotics, anxiolytics, benzodiazepines, acetylcholinesterase inhibitors, memantine
9 Supplement use (e.g., coenzyme Q10, vitamins, creatine), unless stable dose for 6 weeks prior to Screening and with a dose regimen that is not anticipated to change during the study
10. Antiplatelet or anticoagulant therapy within the 28 days prior to Screening or anticipated use during the study. This includes but is not limited to: clopidogrel, dipyridamole, warfarin, dabigatran, rivaroxaban and apixaban. Aspirin is allowed.
11. Oral carbonic anhydrase inhibitors (methazolamide, acetazolamide, benzolamide, topiramate) as they may alter CSF flow dynamics
12. Treatment with amyloid-targeting antibody within the last 3 years prior to Screening
13. Treatment with another IT administered medication within the last 1 year prior to Screening
14. Active infection requiring systemic antiviral or antimicrobial therapy that will not be completed at least 7 days prior to the study drug dosing on Day 1
15. Prior treatment with an siRNA or antisense oligonucleotide (ASO)
16. Any history of gene therapy or cell transplantation or experimental brain surgery
17. Presence of an implanted shunt for the drainage of CSF or an implanted CNS catheter

### Medical Conditions

18. Any condition, including EOAD-related symptoms, that would prevent either writing or performing assessments
19. Attempted suicide, suicidal ideation with a plan that required hospital admission and/or change in level of care within 12 months prior to Screening.
20. Any condition that increases risk of meningitis (e.g., immunodeficient state)
21. History of bleeding diathesis or coagulopathy
22. A medical history of brain or spinal disease that would interfere with the LP process, CSF circulation or safety assessment, including, but not limited to, tumors or abnormalities by MRI or computed tomography (CT), subarachnoid hemorrhage, a suggestion of raised intracranial pressure on MRI or ophthalmic examination, spinal stenosis or curvature, Chiari malformation, hydrocephalus, syringomyelia, tethered spinal cord syndrome, and connective tissue disorders (e.g., Ehlers-Danlos syndrome and Marfan syndrome)
23. History of uncontrolled seizures within the last 6 months prior to Screening
24. Hospitalization for any major medical or surgical procedure involving general anesthesia within 12 weeks of Screening or planned during the trial
25. Clinically relevant hematological, hepatic, cardiac or renal disease or event (e.g., previous acute coronary syndrome within 6 months of Screening). Has other medical conditions or comorbidities which, in the opinion of the Investigator, would interfere with study compliance or data interpretation
26. History of intolerance to IT injection(s)

### Contraception, Pregnancy, and Breastfeeding

27. Is not willing to comply with the contraceptive requirements during the study period
28. Female patient is pregnant, planning a pregnancy, or breast-feeding

### Alcohol Use

29. History of drug/chemical or alcohol abuse, within the last 12 months before Screening, in the opinion of the Investigator
   Exclusion Criteria for Patients Who Transition from Part A to Part B Laboratory Assessments

Patients are excluded from the study if any of the following criteria apply:

### Laboratory Assessments

1. Has any of the following laboratory parameter assessments at Screening:
   a. International normalized ratio (INR)>1.4
   b. Platelet count <100,000/microliter (μL)
   c. Absolute neutrophil count <lower limit of normal (LLN) cells/μL or absolute lymphocyte count <LLN cells/μL
2. Clinically significant ECG abnormalities at Screening, in the opinion of the Investigator, or a Friderica-corrected QT interval (QTcF)>450 msec at Screening
3. Has systolic blood pressure >150 mmHg and/or a diastolic blood pressure >90 mmHg after 10 minutes of rest
4. Has known active human immunodeficiency virus (HIV) infection; or evidence of current or chronic hepatitis C virus (HCV) or hepatitis B virus (HBV) infection
5. Has active severe acute respiratory syndrome coronavirus 2 (SARS-CoV-2) infection

### Prior/Concomitant Therapy

6. Treatment with another investigational drug, biological agent, or device within 6 months of Screening, or 5 half-lives of investigational agent, whichever is longer. Any agent that has received health agency authorization (including for emergency use) by local or regional regulatory authorities is not considered investigational.
7 Use of the following medications is prohibited unless the dose has been stable for at least 12 weeks prior to Screening and the dose regimen is not anticipated to change during the study: antidepressants, antipsychotics, anxiolytics, benzodiazepines, acetylcholinesterase inhibitors, memantine
8. Supplement use (e.g., coenzyme Q10, vitamins, creatine), unless stable dose for 6 weeks prior to Screening and with a dose regimen that is not anticipated to change during the study
9 Antiplatelet or anticoagulant therapy within the 28 days prior to Screening or anticipated use during the study. This includes but is not limited to: clopidogrel, dipyridamole, warfarin, dabigatran, rivaroxaban and apixaban. Aspirin is allowed.
10. Oral carbonic anhydrase inhibitors (methazolamide, acetazolamide, benzolamide, topiramate) as they may alter CSF flow dynamics
11. Treatment with amyloid-targeting antibody within the last 3 years prior to Screening
12. Treatment with another IT administered medication within the last 1 year prior to Screening
13. Active infection requiring systemic antiviral or antimicrobial therapy that will not be completed at least 7 days prior to the study drug dosing on Day 1
14. Prior treatment with an siRNA (except for mivelsiran received in Part A) or antisense oligonucleotide (ASO)
15. Any history of gene therapy or cell transplantation or experimental brain surgery
16. Presence of an implanted shunt for the drainage of CSF or an implanted CNS catheter

### Medical Conditions

17. Attempted suicide, suicidal ideation with a plan that required hospital admission and/or change in level of care within 12 months prior to Screening.
18. Any condition that increases risk of meningitis (e.g., immunodeficient state)
19. History of bleeding diathesis or coagulopathy
20. A medical history of brain or spinal disease that would interfere with the LP process, CSF circulation or safety assessment, including, but not limited to, tumors or abnormalities by MRI or computed tomography (CT), subarachnoid hemorrhage, a suggestion of raised intracranial pressure on MRI or ophthalmic examination, spinal stenosis or curvature, Chiari malformation, hydrocephalus, syringomyelia, tethered spinal cord syndrome, and connective tissue disorders (e.g., Ehlers-Danlos syndrome and Marfan syndrome)
21. History of uncontrolled seizures within the last 6 months prior to Screening
22. Hospitalization for any major medical or surgical procedure involving general anesthesia within 12 weeks of Screening or planned during the trial
23. Clinically relevant hematological, hepatic, cardiac or renal disease or event (e.g., previous acute coronary syndrome within 6 months of Screening). Has other medical conditions or comorbidities which, in the opinion of the Investigator, would interfere with study compliance or data interpretation
24. History of intolerance to IT injection(s)

### Contraception, Pregnancy, and Breastfeeding

25. Is not willing to comply with the contraceptive requirements during the study period
26. Female patient is pregnant, planning a pregnancy, or breast-feeding

### Alcohol Use

27. History of drug/chemical or alcohol abuse, within the last 12 months before Screening, in the opinion of the Investigator
   Discontinuation from Study Drug or Declining Procedural Assessments

Reasons for discontinuation of study drug include any of the following:
Significant protocol deviation; which includes required treatment with prohibited medication per investigator discretion
Adverse event (AE)
Non-adherence to treatment regimen
Pregnancy
Lost to follow-up
Other reason (non-AE)
Study is terminated by the Sponsor

### Treatments and Other Requirements

### Description

Mivelsiran was supplied as a sterile solution formulated in a vehicle comprised of an isotonic aqueous buffer suitable for IT injection. Placebo for Part A consisted of vehicle (isotonic aqueous buffer suitable for IT injection).

### Dose and Administration

Patients are administered mivelsiran or placebo through IT injection, placebo being administered at the same volume as mivelsiran. Prior to study drug administration, a corresponding 20 mL of CSF is collected for analyses using a standard LP collection kit, followed by administration of 20 mL of the study drug. mivelsiran or placebo is administered as a single IT bolus injection pushed over 2-3 minutes.

### Part A Study Drug Dosing and Progression/Escalation

The starting dose of mivelsiran for patients in Cohort 1 was 25 mg. The maximum dose that may be administered in the study was ≤1200 mg.

Four dose level cohorts (Cohorts 1, 2, 3, and 4) are enrolled sequentially, with each patient receiving a single dose of study drug (Table 4—original plan). For each dose escalation, the decision to proceed to dosing the next cohort and the actual dose to be administered are determined by the SRC based on the review of postdose safety and tolerability data through at least Month 1, as well as RBANS score at Month 1, CBC with differential and CSF sAPPα and sAPPβ levels through Day 15, from at least 5 patients for Cohorts 1 and 2 (6 patients/cohort) and at least 6 patients for the remaining cohorts (8 patients/cohort), cumulative safety data (e.g., if a safety signal is observed, then upon SRC recommendation, a de-escalation cohort may be initiated), and available PK and PD data.

The actual dose to be administered may be modified (higher, lower, or the same, but no more than 3-fold higher than the previous dose) from planned doses based on emerging safety, PK, and PD data from preceding cohorts. The actual dose of the optional cohorts does not exceed a 1.5-fold increment from the highest dose previously tested, and no dose exceeds the maximum dose of 1200 mg.

TABLE 4
Part A Dose Cohorts - Original Plan

| | | Volume of | | |
|---|---|---|---|---|
| | Planned Dose | Intrathecal | | |
| | of Study Drug | Dose | Ratio | Number of |
| Cohort | (mg) | (mL) | (mivelsiran:Placebo) | Patients |
| 1 | 25 | 20 | 4:2 | 6 |
| 2 | 75 | 20 | 4:2 | 6 |
| 3 | 225* | 20 | 6:2 | 8 |
| 4 | 600** | 20 | 6:2 | 8 |
| Optional | 900 | 20 | 6:2 | 8 |
| Optional | 1200 | 20 | 6:2 | 8 |
| Optional | ≤1200 | 20 | 6:2 | 8 |
| Optional | ≤1200 | 20 | 6:2 | 8 |

*Cohort 3, originally planned at 225 mg dose, was administered 50 mg mivelsiran
**Cohort 4, originally planned at 600 mg dose, is administered 100 mg mivelsiran.

Both cohort 3 and cohort 4 doses were adjusted due to emerging PD profiles and greater than expected PD observed.

Sentinel pair dosing was performed at the initiation of each new higher dose cohort. The 2 sentinel patients were monitored for AEs for at least 15 days prior to study drug administration to the remaining patients in the respective dose cohort. The SRC determined when to continue dosing within the cohort. The 2 sentinel patients were randomized at 1:1 (mivelsiran:placebo) and the remaining patients were randomized at 3:1 (for Cohorts 1 and 2) or 5:1 (for Cohorts 3 and 4).

Up to 4 optional cohorts were enrolled (Table 4). The optional cohorts contain 8 patients each, randomized at 6:2 (mivelsiran:placebo). Note that if the optional dose level is higher than the previously tested dose levels, the randomization scheme as specified for Cohorts 3-4 is used, including the use of sentinel patients.

### Part B Study Drug Dosing and Progression/Escalation

### Initiation of Drug Dosing

Dosing in Part B may start when the SRC recommends the dose level/dosing frequency for the first Part B cohort, while the follow-up and/or dosing of certain Part A cohort(s) is ongoing.

The SRC first reviews the Part A data to inform Part B dose selection after the availability of data from at least 3 Part A cohorts through at least the Month 3 visit. This defines a minimum dataset from Part A to guide Part B dose selection to include at least 3 cohorts, at least 20 patients enrolled (14 on active treatment), and available safety and PD data through at least 3 months. When a dose level is identified to move forward to Part B, all or a proportion of patients who have completed Part A by that point are assigned to the Part B cohort based on the order of study enrollment.

Table 5 lists the decision-making principles to guide the SRC on the dose selection.

TABLE 5
Decision-making Principles for Dose Selection

| The Recommended Part B Dose Level Should: | | Minimal Data Package Required for SRC Review | |
|---|---|---|---|
| 1. | Be safe and tolerated in Part A | I. | Safety and tolerability learnings for all patients |
| 2. | Not exceed 180 mg and the cumulative annual | | studied in at least 3 Part A cohorts through at |
| | dose should not exceed 360 mg; or not exceed | | least the Month 3 visit. |
| | 225 mg, and the cumulative annual dose should | II. | PD data for all patients studied in at least 3 |
| | not exceed 450 mg. | | Part A cohorts through at least the Month 3 |
| 3. | Doses are not administered more frequently than | | visit. |
| | once every 3 months. | | |
| 4. | Have sufficient PD data (observed or model | | |
| | predicted)a to understand the duration of the | | |
| | effect after single dose and support Part B | | |
| | dosing | | |
| 5. | Produce ≥25% reduction in CSF sAPPα and | | |
| | sAPPβ relative to baseline in ≥3 patients on | | |
| | active treatment, in a given Part A cohort | | |

Abbreviations: CSF = cerebrospinal fluid; PD = pharmacodynamics; sAPPα = soluble amyloid precursor protein alpha; sAPPβ = soluble amyloid precursor protein beta; SRC = Safety Review Committee.

Based on the totality of available PD data from Part A cohorts, PK/PD modeling may support determination of the frequency of the dosing regimen. The minimum interval of dosing is Q3M. If the dosing interval is predicted to be greater than 3 months, the first dose of part B may be administered but redosing can only occur after SRC has approved the dosing frequency.

### Dosing Progression and Escalation

No dose in Part B exceeds the highest dose that is found to have an acceptable safety profile in Part A. The maximum dose administered in Part B does not exceed 225 mg, with no greater than 450 mg cumulative dose given per annum (e.g., maximum regimens of 225 mg q6M). Alternatively, the maximum dose administered in Part B does not exceed 180 mg, with no greater than 360 mg cumulative dose given per annum (e.g., maximum regimens of 180 mg q6M).

### Study Assessments

All assessments on dosing days are to be performed predose, except for postdose PK sample collection, postdose ECG measurements, and neurological assessment.

### Confirmation of AD Diagnosis

Inclusion in the study requires a clinical presentation consistent with MCI or mild dementia due to AD, confirmed with either amyloid PET imaging that shows amyloid plaque positivity or CSF biomarker analysis that indicates underlying AD pathology. Historical results are considered acceptable. If historical results are used for inclusion, amyloid PET must still be performed on Screening or Day −1 in Part A. If historical results being used were from a previous PET scan performed as part of this study within the last 120 days, the patient does not need to have another PET scan at Screening or Day −1 as the prior PET scan can be used for both eligibility and baseline.

### Amyloid Positron Emission Tomography Imaging

An ¹⁸F-labeled amyloid PET agent is a radioactive diagnostic agent for PET imaging of the brain to estimate β-amyloid neuritic plaque density in adult patients with cognitive impairment who are being evaluated for AD and other causes of cognitive decline.

If historical CSF biomarker result or historical amyloid PET positivity is not available, positive amyloid PET (by visual read) must be obtained during Screening for eligibility (i.e., positive amyloid PET signal is required for study inclusion in Part A or for new patients entering Part B). When historical confirmation of AD is available (either via CSF laboratory result or historical amyloid PET positivity), the amyloid PET obtained on Screening or Day −1 only has a quantitative analysis (standardized uptake value ratios (SUVR), centiloids) performed and not contribute to study eligibility.

In the event that a patient has both historical amyloid PET and historical CSF biomarker results available and the results are incongruent (i.e., positive CSF result but negative PET result, or vice versa), the positive result can be used to support eligibility for the study.

### CSF Biomarker Analysis

Measurement of Aβ42, total tau (t-Tau), and phosphorylated tau (p-Tau) in the CSF is useful in the differential diagnosis of AD from other causes of cognitive impairment. For this study, the CSF biomarker analysis is considered positive for AD pathology if 1) all 3 biomarkers are abnormal, 2) the p-Tau/Aβ42 ratio (i.e., Aβ42 to p-tau index, PTI) is abnormal, or 3) the t-tau/Aβ42 ratio (i.e., Aβ42 to t-tau index, ATI) is abnormal.

### Mini Mental State Examination

Mini Mental State Examination (MMSE) is an instrument for assessing cognitive function in elderly patients (Folstein et al. (1975) J. Psychiatr. Res., 12(3):189-198). It is a 30-question (30-point) test to assess orientation, attention, memory, language, and visual-spatial skills. The instrument is divided into 2 sections. The first section measures orientation, memory, and attention with a maximum score of 21 points. The second section measures the ability of the patient to name objects, follow written/verbal commands, write a sentence and copy figures with a maximum score of 9 points. The MMSE has a total score range of 0 to 30, with lower scores indicating a higher level of impairment. The MMSE should be performed at Screening to assess the patient's cognitive function and a score >20 is required for study inclusion. For patients who have participated in Part A, MMSE score is not considered for Part B eligibility.

### Clinical Dementia Rating: Global Score

Clinical Dementia Rating (CDR) is a semi-structured interview performed with the patient and study partner (caregiver/informant) that provides an index of global functioning (Morris (1993) Neurology, 43(11):2412-2414). The assessment instrument yields a Global score and a Sum of Boxes (CDR-SB) score. The CDR Global score is computed via an algorithm where the global functioning is rated on a 3.0 scale as follows: 0, no impairment; 0.5, questionable impairment; 1, mild impairment; 2, moderate impairment; and 3, severe impairment.

At Screening, the CDR Global score must be 0.5 or 1.0 for inclusion in the study. For patients who have participated in Part A, CDR score is not considered for Part B eligibility.

### Effect of Mivelsiran on Clinical Progression

The effect of mivelsiran on clinical progression is evaluated by the change from baseline of the cognitive, functional, and neuropsychiatric measures.

Cognitive, functional, and neuropsychiatric assessments should be performed at approximately the same time and same order of each study visit to minimize potential variability. If possible, each assessment should be performed by the same rater at each visit. When performed on the same day as medical procedures or neuroimaging, if possible, assessments should be performed before the procedures or neuroimaging which can potentially cause abnormal performance on the tests.

### Cognitive and Functional Measures

The following cognitive and/or functional assessments can be used to monitor treatment effects including expected and unexpected cognitive changes in patients on treatment.

Repeatable Battery for the Assessment of Neuropsychological Status (RBANS): This assessment is a brief neuropsychological screening for neurocognitive status. The 12 items on RBANS assess 5 cognitive domains: immediate memory, visuospatial/constructional abilities, language, attention, and delayed memory. The 5 domain scores are combined as a total scale score implying a second-order factor structure. The possible values for the RBANS scores at the item, domain, and scale level are 0 to 89, 40 to 154, and 40 to 160, respectively; and the score decreases with increased impairment.

Alzheimer's Disease Assessment Scale-Cognitive Subscale 13-item Version (ADAS-Cog13): This test is a rater administered instrument which contains 13 tasks, including subject-completed tests and observer-based assessments. It evaluates the severity of dysfunction in both cognitive and noncognitive behavioral profiles (Rosen et al. (1984) Am. J. Psychiatry, 141(11):1356-1364). The test evaluates 13 areas that are typically impaired in AD: word recall, naming objects and fingers, commands, constructional praxis, ideational praxis, orientation, word recognition, language, spoken language, word finding difficulty, remembering, number cancellation, and delayed word recall measures (Mohs et al. (1997) Alzheimer Dis. Assoc. Disord., 11 Suppl 2: S13-S21). The Alzheimer's Disease Assessment Scale-Cognitive Subscale 13-item version (ADAS-Cog13) allows for better discrimination of functional differences for individuals with mild AD as compared to ADAS-Cog11, and considered the gold standard for assessing efficacy of antidementia treatments. The ADAS-Cog13 score ranges from 0 to 85 points, with higher scores indicating greater dysfunction.

Clinical Dementia Rating (CDR): This assessment is a semi-structured interview performed with the patient and the study partner (caregiver/informant) that provides an index of global functioning (Morris (1993) Neurology, 43(11):2412-2414). The assessment instrument yields both a Global score and a Clinical Dementia Rating Sum of Boxes (CDR-SB) score. The test includes assessment of 6 domains: memory, orientation, judgment and problem solving, community affairs, home and hobbies, and personal care; and higher scores indicate greater disease severity. For CDR-SB, each domain (except for personal care, a 4-point scale only) is rated on a 5-point scale. The 6 domains are summed to create a CDR-SB score of 0-18, and the score increases with degree of dementia (normal=0, mild dementia=4.5-9, severe dementia=16-18).

The CDR Global score, determined at Screening and calculated based on computer algorithm using domain scores as inputs, differs from the calculation of CDR-SB score as described above. The increased range of values in the CDR-SB score allows for better tracking of changes within and between stages of dementia severity. [O'Bryant 2008]. CDR may have potential for discriminating between patients with MCI and very early AD (who are assigned a global CDR score of 0.5).

### Neuropsychiatric Measures

Neuropsychiatric Inventory Questionnaire (NPI-Q): This is a 12-question survey to be completed by the caregiver/study partner. The questionnaire assesses the presence and severity of behavioral disturbances: delusions, hallucinations, agitation/aggression, dysphoria/depression, anxiety, euphoria/elation, apathy/indifference, disinhibition, irritability/lability, aberrant motor, nighttime behavior, and appetite/eating.

Initial responses to each domain question are “Yes” (present) or “No” (absent). If the response to the domain question is “No”, the caregiver/study partner goes to the next question. If “Yes”, the caregiver/study partner then rates both the severity of the symptoms present within the last month on a 3-point scale and the associated impact of the symptom manifestations (i.e., caregiver distress) using a 5-point scale. A higher score represents more severe neuropsychiatric impairment and/or increased caregiver distress. The Neuropsychiatric Inventory Questionnaire (NPI-Q) provides symptom severity and distress ratings for each symptom reported, with total Severity and Distress scores reflecting the sum of individual domain scores.

Pittsburgh Sleep Quality Index (PSQI): This is a self-rated 19-item questionnaire which assesses sleep quality and disturbances. A global score is obtained from 7 component scores: subjective sleep quality, sleep latency, sleep duration, habitual sleep efficiency, sleep disturbances, use of sleeping medication, and daytime dysfunction. The sum of the individual components gives a Global PSQI Score.

### Physician's Impression of Health Status Change

The Alzheimer's Disease Cooperative Study-Clinical Global Impression of Change (ADCS-CGIC) is assessed. The ADCS-CGIC score is intended as a measure of clinically meaningful change as viewed by an independent skilled and experienced clinician based on direct interview of the patient and the study partner (caregiver/informant). The ADCS-CGIC focuses on clinicians' observations of change in the patient's cognitive, functional, and behavioral performance since the beginning of a trial, but unlike a targeted symptom scale, it considers a patient's overall function.

### Pharmacodynamic Assessments

CSF and blood samples are collected for monitoring the pharmacodynamic (PD) effect (change in sAPPα and sAPPβ levels), biomarkers of disease activity, and inflammatory biomarkers (CSF only). The exploratory biomarkers of disease activity include but are not limited to CSF biomarkers (e.g., Aβ40, Aβ42, NfL, NfH, APOE, neurogranin, SNAP25, S100B, t-Tau, and p-Tau species) and plasma biomarkers (sAPPα, sAPPβ, Aβ40, Aβ42, NfL, t-Tau, and p-Tau species). Inflammatory biomarkers include but are not limited to C3, C5, IL-6, TNF, IL-1, MCP-1, and YKL-40. These measurements are collected and analyzed centrally. Blood and CSF samples for PD assessments must be collected before study drug administration.

In Part A, patients whose CSF sAPPα and sAPPβ levels have not returned to ≥75% of the Day 1 sAPPα and sAPPβ levels for at least 2 consecutive visits by the Month 6 visit, CSF and blood samples are collected for PD assessment q2M during the Follow-up Period until CSF sAPPα and sAPPβ levels return to ≥75% of Day 1 predose levels for 2 consecutive visits (Drug Washout), or up to Month 12, whichever is earlier.

### Biomarkers for Disease Activity and Inflammation

The effect of mivelsiran on CSF levels of exploratory biomarkers of disease activity, including but not limited to Aβ40, Aβ42, neurofilament light chain protein (NfL), neurofilament heavy chain protein (NfH), apolipoprotein E (APOE), neurogranin, synaptosome associated protein 25 (SNAP25), S100 calcium binding protein B (S100B), t-Tau, and p-Tau, are monitored. Plasma levels of Aβ40, Aβ42, NfL, t-Tau, and p-Tau species are also measured. In addition, inflammatory biomarkers including but not limited to complement component 3 (C3), complement component 5 (C5), interleukin-6 (IL-6), interleukin-1 (IL-1), tumor necrosis factor (TNF), monocyte chemoattractant protein-1 (MCP-1), chitinase-3-like protein 1 (YKL-40), are monitored. These biomarkers are associated with amyloidopathy, tauopathy, neuroinflammation, and neurodegeneration, collectively allowing for the characterization of underlying disease pathology.

### Pharmacokinetic Assessments

Blood, CSF, and urine (Part A only) samples were collected for the assessment of PK parameters and potential metabolite analysis. Postdose pharmacokinetic (PK) sampling time points in Part B mirrors Part A PK sampling time points and may be adjusted based on Part A PK results. The concentration of mivelsiran and its potential major metabolites is determined using validated assays. Additionally, CSF or plasma samples may also be used for exploratory PK analysis using a quantitative polymerase chain reaction assay for determining mivelsiran concentrations.

### Safety Assessments

The assessment of safety during the study consists of the surveillance and recording of AEs including SAEs, recording of concomitant medication, medical history, and measurements of vital signs, weight, ECG findings, laboratory tests, CSF laboratory assessment, cognitive and suicide severity assessment, and neuroimaging assessments. Clinically significant abnormalities observed during the physical examination and full standard neurological assessment are recorded as AEs. Multiple clinical and laboratory assessments provide important safety information in real time.

### Vital Signs

Vital signs are measured and include blood pressure, heart rate, body temperature, and respiratory rate. Vital signs are to be collected prior to dosing at dosing visits.

### Weight

Body weight measurements in kilograms are collected.

### Physical Examination

Full physical examinations are conducted and should be conducted prior to dosing. Full physical examinations include the examination of the following: general appearance; head, eyes, ears, nose and throat; respiratory, cardiovascular, gastrointestinal, musculoskeletal, and dermatological systems; thyroid; lymph nodes; and neurological status.

### Neurological Assessment

Neurological assessment is conducted and includes a thorough assessment of mental status, meningeal signs, cranial nerves, motor system examination including strength, tone, deep tendon reflexes, and plantar reflexes, sensory systems, gait, balance, and coordination. The neurological examination also includes non-dilated fundoscopic assessment.

For Part A and Part B, full neurological assessment should be performed approximately 3 hours postdose and prior to discharge on Day 2.

### Electrocardiogram

The 12-lead ECGs reporting rhythm, ventricular rate, RR interval, PR interval, QRS duration, and QT interval and Fridericia corrected QT interval (QTcF) are obtained; these are also collected by 24-hour Holter monitoring. Continuous 12 lead digital ECG data are recorded on a 24-hour 12-lead Holter monitor for assessment of QTc interval from 24 hours before dosing to 24 hours after dosing. Triplicate 12-lead ECGs are extracted from the continuous recording.

### Clinical Laboratory Assessments

Clinical laboratory assessments performed by a central laboratory are listed in Table 6 and are collected according to a specified schedule.

TABLE 6
Clinical Laboratory Assessments

Hematology

Complete blood count with differential, including platelet counts (also
performed by local laboratory before LP procedure)
Peripheral blood smear (if absolute neutrophil count <1000 cells/μL and
not previously performed by the Investigator)

| | |
|---|---|
| Serum Chemistry | |
| Sodium | Potassium |
| BUN | Phosphate |
| Uric acid | Albumin |
| Total protein | Calcium |
| Glucose | Bicarbonate |
| Creatinine and eGFR | Chloride |
| Liver Function Tests | |
| AST | ALP |
| ALT | Bilirubin (total and direct) |
| GGT | |
| Urinalysis | |
| Visual inspection for appearance | Bilirubin |
| and color | |
| pH (dipstick) | Nitrite |
| Specific gravity | RBCs |
| Ketones | Urobilinogen |
| Albumin | Leukocytes |
| Glucose | Microscopy (if clinically |
| | indicated) |
| Protein | |
| Coagulation | |
| Prothrombin time | International normalized |
| | ratio aPTT |
| Immunogenicity | |
| Antidrug antibodies | |
| Pregnancy Testing/FSH Screening | |

β-human chorionic gonadotropin (females of child-bearing potential only)
FSH (postmenopausal women only)

### Immunogenicity

Blood samples are collected to evaluate incidence and titer of antidrug antibodies (ADA) against mivelsiran.

### Pregnancy Testing

A pregnancy test for β-human chorionic gonadotropin is performed for females of child-bearing potential. A blood sample is drawn at Screening to measure the levels of follicle stimulating hormone in order to confirm post-menopausal status in all women self-reported to be postmenopausal.

### Cerebrospinal Fluid Safety Laboratory Assessment

The CSF samples that are collected for PD analysis are also evaluated by the local laboratory for the number of red and white blood cells (and differential), and protein and glucose levels.

### Neuroimaging Evaluations

The effect of mivelsiran on disease activity is evaluated by the following neuroimaging evaluations.

Amyloid PET imaging: An ¹⁸F-labeled amyloid PET agent is a ligand that binds to extracellular fibrillar amyloid plaque and allows for the characterization of existing brain amyloid load by ¹⁸F-labeled amyloid PET imaging. This neuroimaging method provides qualitative and quantitative analysis of parenchymal amyloid plaque burden in patients with early symptomatic AD. The quantitative assessment measures baseline amyloid plaque burden and can potentially assess plaque reduction in the brain by mivelsiran. Absence of amyloid PET signal on a visual read indicates sparse or no amyloid plaque and thus the absence of AD pathology. Therefore, amyloid PET imaging serves as a screening tool to confirm the AD diagnosis.

If historical result is used for study inclusion, baseline amyloid PET must still be performed on Screening or Day −1 of Part A and is compared to the result at Month 6.

In the case where the historical PET being used is an amyloid PET scan performed as part of this study, it is unnecessary to repeat amyloid PET scan at Screening or Day −1 if no more than 120 days have elapsed since the previous PET scan.

Amyloid PET imaging on Day 1 of Part B is omitted if it has been performed within the last 3 months (i.e., during Part A).

Non-contrast brain MRI scan: Volumetric magnetic resonance imaging (vMRI) detects brain atrophy which serves as an indicator of neurodegeneration. Brain MRI scans are also performed and evaluated for the presence of amyloid-related imaging abnormalities (ARIA) throughout the study until 6 months after the last dose of mivelsiran.

Tau PET imaging (Optional, Baseline only, Part A and/or Part B): ¹⁸F-labeled tau PET agent is a radioactive diagnostic agent indicated for PET imaging of the brain to estimate the density and distribution of aggregated tau neurofibrillary tangles in adult patients with cognitive impairment who are being evaluated for AD.

Non-contrast spine MRI scan (Part A and new patients for Part B): Spinal MRI characterizes local neuroanatomical abnormalities that can affect the study drug biodistribution. ¹⁸F-labeled amyloid PET scans result in typical effective radiation doses of approximately 9.0 mSv per study for an adult. ¹⁸F-labeled tau PET scans result in typical effective radiation doses of approximately 10.7 mSv per study for an adult. The maximum recommended allowed effective dose for a research patient in a single year is 50 mSv (refer to 21 Code of Federal Regulations [CFR] 361.1, Radioactive drugs for certain research uses). The maximum number of PET scans in the study is 4 amyloid PET scans and 2 tau PET scans, bringing the maximum total radiation exposure to approximately 60 mSv over a period of 2 years. The maximum number of PET scans in 1 year in the study is 2 amyloid PET scans and 2 tau PET scans, which would result in an approximate dose of 40 mSv, or approximately 80% of the recommended maximum allowed dose.

### Suicide Severity Assessment

Suicide severity assessment is performed at approximately the same time of the study visit to minimize potential variability. If possible, it should be performed by the same rater at each visit.

Columbia—Suicide Severity Rating Scale (C-SSRS): This rating scale is designed to quantify the severity of suicidal ideation and behavior using 4 subscales. Severity subscale is rated on a 5-point ordinal scale. Intensity subscale comprises 5 items: frequency, duration, controllability, deterrents, and reason for ideation, with each rated on a 5-point ordinal scale. Behavior subscale includes actual, aborted, and interrupted attempts, preparatory behavior, and non-suicidal self-injurious behavior and is rated on a nominal scale. Lethality subscale assesses actual attempts and is rated on a 6-point ordinal scale. If actual lethality is 0, potential lethality of attempts is rated on a 3-point ordinal scale (Posner et al. (2011) 18FAm. J. Psychiatry, 168(12):1266-1277). A higher score indicates more intense suicidal ideation and greater risk.

### Statistics

A Statistical Analysis Plan (SAP) is finalized before database lock. The plan details the implementation of the statistical analyses in accordance with the principle features stated in the protocol.

### Determination of Sample Size

This is a Phase 1 study to investigate the safety, tolerability, PK, and PD of mivelsiran. The sample sizes for both Part A and B are not based on formal statistical testing. The planned enrollment for the study is up to 60 patients in Part A and Part B. Based on previous clinical data, the SD for percent reduction in CSF sAPPβ ranged from 3% to 10% (Timmers et al. (2018) 18FAlzheimers Res. Ther., 10(1):85). Hence, assuming an SD of 10%, the half-width of the 95% confidence interval (CI) for percent reduction of CSF sAPPβ is estimated to be 8% for a minimum group size of 6 patients.

Part B includes at least 1 planned cohort and up to 3 cohorts. A minimum of 6 patients are enrolled in each Part B dosing cohort.

### Statistical Methodology

Statistical analyses are primarily descriptive. No formal hypothesis testing is conducted. Summary tables present results by dose group (e.g., mivelsiran dose level in each cohort, combined mivelsiran, and combined placebo across dose cohorts) in Part A and for each mivelsiran dosing regimen in Part B. Descriptive statistics are presented for continuous variables, and frequencies and percentages are presented for categorical and ordinal variables. Percentages are based on the number of non-missing values in a dose group or dosing regimen.

Analyses are performed using SAS® (Version 9.4 or higher).

### Results

### Patient Cohorts

As of Sep. 20, 2023, 6 patients per cohort were enrolled and randomized 2:1 to receive intrathecal administration of mivelsiran or placebo in 25 mg (cohort 1) and 75 mg (cohort 2) single dose cohorts. Baseline characteristics of cohorts 1 and 2 are shown in Table 8a. Further, As of Sep. 20, 2023, 8 patients were enrolled and randomized 3:1 to receive intrathecal administration of mivelsiran or placebo in a 50 mg single dose cohort (cohort 3). Baseline characteristics of cohorts 1-3 including the mean duration in the study are shown in Table 8b.

As of Nov. 16, 2023, 20 patients (mean [range] age, 61.3 [53-73] years; 60.0% male; 80.0% white) were randomized to mivelsiran or placebo in the 25 mg (N=6, 2:1 randomization), 50 mg (N=8, 3:1), and 75 mg (N=6, 2:1) cohorts.

As of February 2024, the study included the cohorts of patients as summarized in Table 7.

TABLE 7
Part A Dose Cohorts - as of February 2024

| | | Number of | Number of |
|---|---|---|---|
| | | patients | patients |
| | | receiving | receiving |
| Cohort | Description | mivelsiran | placebo |
| 1 | 25 mg single dose | 4 | 2 |
| 2 | 75 mg single dose | 4 | 2 |
| 3 | 50 mg single dose | 6 | 2 |
| 4 | 100 mg single dose | 6 | 2 |
| 5 | optional cohort, 75 mg single dose | 6 | 2 |
| 6 | optional cohort, 35 mg single dose | 6 | 2 |
| 7 | 150 mg single dose | 6 | 2 |

### Blinded Safety Summary

All adverse events (AEs) by the initial data cut-off on Jan. 17, 2023 are summarized in Table 9a. Pooled AEs for cohorts 1-3 as of Jun. 29, 2023 are summarized in Tables 9b and 9c. Pooled AEs for cohorts 1-3 as of Sep. 20, 2023 are summarized in Tables 9d, 9e, and 9f. As of Sep. 20, 2023, AEs were reported in 19 patients (95.0%). All AEs were mild or moderate, with many AEs reported having been deemed related to lumbar puncture procedure by the investigator. One individual in the 50 mg or placebo cohort had two mild AEs (post-LP headache and post-LP nausea) that were deemed both study drug-related and procedure-related by the investigator (see Table 9d); both events resolved on the same day. As of Sep. 20, 2023, 60% of patients have experienced AEs that were deemed related to lumbar puncture by the investigator. These AEs include procedural headache (40% of patients), headache (15%), back pain (10%), vomiting (10%), procedural pain (10%), dizziness (5%), injection site swelling (5%), neck pain (5%), presyncope (5%), procedural nausea (5%), puncture site pain, (5%) and syncope (5%). No deaths or suspected unexpected serious adverse reactions (SUSARs) occurred.

As of Feb. 23, 2024, the majority of AEs continued to be mild or moderate in severity, with no deaths or SUSARs. One (1) patient had severe AE on study in December 2023 for post-LP syndrome, which was not related to the study drug. This event was reported as non-serious; severe AE, related to LP, not related to study drug.

First AECI (adverse event of clinical interest) was reported in January 2024, not related to the study drug. The principal investigator (PI) assessed that this was progression of AD related motor abnormalities, not related to the study drug.

As of Feb. 23, 2024, there were no concerning trends related to amyloid-related imaging abnormalities-hemosiderosis/microhemorrhages (ARIA-H) treatment emergent adverse events (TEAEs).

### Safety Labs

The CSF safety assessments were largely within normal limits and there was no evidence of neuroinflammation due to mivelsiran. For example, as shown in FIG. 2 CSF WBD counts have not shown any concerning trends. As shown in FIG. 3, the CSF protein levels have not shown any concerning trends. Routine laboratory assessments, including hematology, serum chemistry, liver function, urinalysis, and coagulation, did not reveal any trends of concern.

### Cognitive Scales

CDR-SB, RBANS, or ADAS-Cog-13 cognitive testing did not show any trends or concern or safety signals over the study time period.

### Changes in CSF Neurofilament Light Chain (NfL) Levels

Preliminary data for the exploratory biomarker neurofilament light chain (NfL, marker of neurological damage and disease activity), including CSF NfL mean and median % changes from baseline, did not reveal any trends of concern.

### Reduction in CSF Soluble APPα and APPβ Levels

Rapid and sustained reductions in sAPPα and sAPPβ were observed following a single dose of mivelsiran in patients with EOAD. Dose-dependent reductions of soluble APPα and APPβ (sAPPα and sAPPβ) levels in cerebrospinal fluid at day 15 were observed following a single dose of mivelsiran, with mean reductions from baseline of 55% (sAPPα) and 69% (sAPPβ), and maximum reductions of 71% (sAPPα) and 83% (sAPPβ) in the 75 mg cohort (n=4) (Table 10).

Peak mean (±SEM) reduction in sAPPα was 69% (±9.55) for 75 mg dose occurring at Month 2, with a maximum individual reduction of 84% was observed. Reduction in sAPPα was sustained, with a 56% (±7.53) mean reduction and a 31% (±7.6) mean reduction 6 months after a single 75 mg dose and a single 50 mg dose, respectively, and a 33% (±6.1) mean reduction 10 months after a single 75 mg dose.

Peak mean (±SEM) reduction in sAPPβ was 82% (±6.26) for 75 mg dose occurring at Month 2, with a maximum individual reduction of 90% was observed. Reduction in sAPPβ was sustained, with a 65% (±9.24) mean reduction and a 48% (±5.5) mean reduction 6 months after a single 75 mg dose and a single 50 mg dose, respectively, and a 39% (±11.5) mean reduction 10 months after a single 75 mg dose.

In the 75 mg single dose cohort, the median reductions from baseline of about 60-80% sAPPα (FIG. 4) and about 75-85% sAPPβ (FIG. 5) were observed over 6 months, the median reduction from baseline of sAPPα of 40% or greater (FIG. 4) and the median reduction from baseline of sAPPβ of about 45% or greater (FIG. 5) were observed over 10 months, and the median reduction from baseline of sAPPα of 20% or greater (FIG. 4) and the median reduction from baseline of sAPPβ of about 20% or greater (FIG. 5) were observed over 12 months. A median decrease in both sAPPα and sAPP of greater than 70% was sustained for at least 3 months, a median decrease in both sAPPα and sAPPβ of 60% or greater was sustained for at least 6 months, a median decrease in both sAPPα and sAPPβ of 40% or greater was sustained for at least 10 months, and a median decrease in both sAPPα and sAPPβ of 20% or greater was sustained for at least 12 months after single dose administration of 75 mg mivelsiran.

In the 50 mg single dose cohort, the median reductions from baseline of about 55-60% sAPPα (FIG. 4) and about 70-75% sAPPβ (FIG. 5) were observed over 3 months, the median reductions from baseline of about 30-60% sAPPα (FIG. 4) and about 60-75% sAPPβ (FIG. 5) were observed over 6 months, and the median reductions from baseline of about 15% sAPPα (FIG. 4) and about 40% sAPPβ (FIG. 5) were observed over 10 months.

Percent (%) reduction or % decrease referenced herein was measured as compared to the baseline level in the subject before administration of mivelsiran. Table 11 summarizes ongoing data capture at Month 6 with mivelsiran administered at 50 mg and Month 10 with mivelsiran administered at 75 mg, and demonstrates rapid and sustained reduction from baseline in CSF.

### Reduction in CSF Aβ42 and Aβ40 Levels

Following administration of mivelsiran, robust reductions in CSF of exploratory disease-related biomarkers Aβ42 and Aβ40 were observed, for example as shown in FIGS. 6A-6C and 7A-7C. Aβ42 and Aβ40 are the soluble forms of the amyloidogenic peptides that aggregate into amyloid deposits in AD. As shown in FIGS. 6A and 7A, after a single dose of 50 mg mivelsiran, mean (±SEM) reductions in CSF Aβ42 and Aβ40 were 51.9% (±6.8) and 69.8% (±7.0), respectively at 2 months, and about 40% and about 50%, respectively at 6 months. After a single dose of 75 mg mivelsiran, mean (±SEM) reductions in CSF Aβ42 and Aβ40 were 48.9% (±7.7) and 70.6% (±9.3), respectively at 2 months, and about 22% and about 50%, respectively at 6 months. Percent (%) decrease referenced herein was measured as compared to the baseline level in the subject before administration of mivelsiran.

Table 12 summarizes ongoing data capture at Month 6 of sustained reductions from baseline in CSF Aβ42 and Aβ40 levels.

As shown in FIGS. 8A-8B and 9A-9B, Aβ42 and Aβ40 levels in plasma demonstrated similar trends to those in CSF. As shown in FIGS. 8A and 9A, after a single dose of 75 mg mivelsiran, mean reductions in CSF Aβ42 and Aβ40 were about 25% and about 30%, respectively at 2 months, and about 35% and about 30%, respectively at 4 months. Percent (%) decrease referenced herein was measured as compared to the baseline level in the subject before administration of mivelsiran.

### Summary and Conclusion

This is the first clinical study to show successful reduction in sAPPα and sAPPβ with a genetic therapy, demonstrating target engagement of APP. The RNAi agents of the present invention have demonstrated surprising and superior results as compared to other anti-sense oligonucleotides that have been tested in human clinical studies. As shown above, patients administered mivelsiran experienced dose-dependent, rapid, and sustained reduction in cerebrospinal fluid of both soluble APPα (sAPPα) and APPβ (sAPPβ), with maximum reduction of 84% and 90%, respectively. Reduction in sAPPα and sAPPβ was sustained, with a 56% (±7.53) mean reduction and a 65% (±9.24) mean reduction, respectively, 6 months after a single 75 mg dose, and a 33% (±6.1) mean reduction and a 39% (±11.5) mean reduction, respectively, 10 months after a single 75 mg dose. The median reductions from baseline of about 60-80% (sAPPα) and about 75-85% (sAPPβ) were observed over 6 months, and the median reduction from baseline of sAPPα of 40% or greater and the median reduction from baseline of sAPPβ of about 45% or greater were observed over 10 months, following a 75 mg mivelsiran single dose administration. The RNAi agents provided herein produced these results with only a single dose without a loading dose, in contrast to typical antisense oligonucleotides (ASOs), which typically require loading doses to achieve inhibitory effects.

Moreover, the RNAi agents of the present invention produced a knockdown of the target (APP) in the CNS that is greater than has been possible with other CNS-targeted anti-sense oligonucleotides. For example, a knockdown of about 33% at 12 months with a 100 mg dose was observed in a phase 1-2 trial of tofersen, an antisense oligonucleotide targeting superoxide dismutase 1 (SOD1), in amyotrophic lateral sclerosis (ALS) patients (Miller, T, et al. (2020) New England J. Med. 383(2):109-119). In contrast, the APP dsRNA agents of the present invention achieved a higher sustained knockdown of about 80% at a lower dose of 75 mg.

Surprisingly, the APP dsRNA agents provided herein have a long knockdown duration of at least 4 months and the knockdown is achieved at lower doses than was expected at the start of the trial. The single dose of 75 mg surprisingly had a sustained knockdown effect of almost 80% on sAPPα and sAPPβ lasting at least out to four months post-administration and the study is still ongoing. Overall, the APP dsRNA agents demonstrate superior properties in dosing, knockdown, and duration compared to other CNS-targeted antisense oligonucleotides that have been tested.

CNS administration of antisense oligonucleotides can result in side effects such as neurotoxicity and neuroinflammation (Hagedorn, P. H., et al. (2022) Nucleic Acid Therapeutics, 32(3):151-162; Goyenvalle, A., et al. (2023) Nucleic Acid Therapeutics, 33(1):1-16). Importantly, these side effects have not been observed in the clinical studies of the APP dsRNA agents of the present invention.

In this ongoing Phase 1 single dose study, as of Feb. 23, 2024, mivelsiran remains generally well tolerated with the majority of AEs being mild or moderate in severity, with no deaths or SUSARs, and has produced robust, durable reductions in CSF soluble APP and downstream Aβ, a key protein implicated in progression of Alzheimer's disease. These interim results support further evaluation of mivelsiran in patients with EOAD. The Part B multi-dose, open-label study is ongoing. In all studies, study parameters described herein, including the levels of sAPPα and sAPPβ, exploratory biomarkers (e.g., Aβ42, Aβ40), inflammatory markers, CSF safety labs, and clinical progression assessments are assessed.

TABLE 8a
Baseline characteristics for cohorts 1 and 2 as of Sep. 20, 2023

| Baseline Characteristics | All Patients (N = 12) | |
|---|---|---|
| Mean age, years (range) | 60.6 | (54-73) |
| Male, n (%) | 9 | (75) |
| Race, n (%) | | |
| White | 9 | (75) |
| Asian | 2 | (17) |
| Black/African American | 1 | (8) |
| Mean BMI, kg/m² (SD) | 26.2 | (3.4) |

TABLE 8b
Baseline characteristics for cohorts 1-3 as of Sep. 20, 2023

| | |
|---|---|
| | All Patients |
| Baseline Characteristics | (N = 20) |

| | | |
|---|---|---|
| Mean age, years (range) | 61.3 | (53-73) |
| Male, n (%) | 12 | (60.0) |
| Race, n (%) | | |
| White | 15 | (75.0) |
| Asian | 3 | (15.0) |
| Black/African American | 1 | (5.0) |
| CDR ® global score, n (%) | | |
| 0.5 | 16 | (80.0) |
| 1.0 | 4 | (20.0) |
| Mean MMSE (SD) | 23.6 | (2.4) |
| Mean BMI, kg/m² (SD) | 25.9 | (3.5) |
| Time from randomization, months, mean (SD) | | |
| Cohort 1 (mivelsiran 25 mg or Placebo, n = 6) | 14.8 | (1.7) |
| Cohort 2 (mivelsiran 75 mg or Placebo, n = 6) | 9.9 | (1.2) |
| Cohort 3 (mivelsiran 50 mg or Placebo, n = 8) | 6.6 | (1.3) |

BMI, body mass index;
CDR ®, clinical dementia rating;
MMSE, Mini Mental State Examination;
SD, standard deviation

TABLE 9a
All adverse events for cohorts 1 and 2 as of Jan. 17, 2023

| | All Patients (N = 12) | |
|---|---|---|
| Adverse Events | n (%) | |
| At least one adverse event | 9 | (75) |
| Back pain | 2 | (17) |
| Headache | 2 | (17) |
| Post lumbar puncture syndrome | 2 | (17) |
| Syncope | 2 | (17) |
| Constipation | 1 | (8) |
| Hemorrhoids | 1 | (8) |
| Injection site pain | 1 | (8) |
| Procedural pain | 1 | (8) |
| ALT increased | 1 | (8) |
| ARIA-microhemorrhages and | 1 | (8) |
| hemosiderin deposits | | |
| Presyncope | 1 | (8) |
| Vomiting | 1 | (8) |

ALT, alanine aminotransferase; ARIA, amyloid-related imaging abnormality

TABLE 9b
Pooled adverse event summary for cohorts 1-3 as of Jun. 29, 2023

| | mivelsiran | mivelsiran | mivelsiran |
|---|---|---|---|
| | 25 mg or | 50 mg or | 75 mg or |
| | PBO | PBO | PBO |
| | (N = 6, | (N = 8, | (N = 6, |
| N (%) | PY = 4.1) | PY = 2.8) | PY = 3.6) |
| At least one mild AE | 5 (83.3) | 6 (75.0) | 4 (66.7) |
| At least one moderate AE | 4 (66.7) | 4 (50.0) | 3 (50.0) |
| At least one serious AE | 0 | 0 | 0 |
| Death | 0 | 0 | 0 |
| At least one AE related | 4 (66.7) | 6 (75.0) | 2 (33.3) |
| to LP | | | |

PBO, placebo;
PY, patient years

TABLE 9c
Pooled adverse events by event type
for cohorts 1-3 as of Jun. 29, 2023

| | mivelsiran | | mivelsiran | mivelsiran |
|---|---|---|---|---|
| | 25 mg or | | 50 mg or | 75 mg or |
| N (%) of events | PBO | | PBO | PBO |
| occurring in ≥10% | (N = 6, | | (N = 8, | (N = 6, |
| of all patients | PY = 4.1) | | PY = 2.8) | PY = 3.6) |
| At least one adverse | 6 | (100.0) | 7 (87.5) | 5 (83.3) |
| event | | | | |
| Post lumbar puncture | 3 | (50.0) | 5 (62.5) | 0 |
| syndrome | | | | |
| Back pain | 2 | (33.3) | 1 (12.5) | 1 (16.7) |
| Presyncope | 0 | | 1 (12.5) | 2 (33.3) |
| Syncope | 2 | (33.3) | 0 | 1 (16.7) |
| Headache | 0 | | 1 (12.5) | 1 (16.7) |
| Nasopharyngitis | 0 | | 0 | 2 (33.3) |
| Vomiting | 2 | (33.3) | 0 | 0 |

PBO, placebo;
PY, patient years

TABLE 9d
Pooled adverse event summary for cohorts 1-3 as of Sep. 20, 2023

| | mivelsiran | | mivelsiran | mivelsiran | |
|---|---|---|---|---|---|
| | 25 mg or | | 50 mg or | 75 mg or | |
| | PBO | | PBO | PBO | |
| Patients with events, | (N = 6, | | (N = 8, | (N = 6, | |
| n (%) | PY = 7.4) | | PY = 4.4) | PY = 4.9) | |
| At least one AE | 6 | (100.0) | 7 (87.5) | 6 | (100.0) |
| Related to study drug | 0 | | 1 (12.5) | 0 | |
| At least one moderate | 4 | (66.7) | 5 (62.5) | 4 | (66.7) |
| AE | | | | | |
| Related to study drug | 0 | | 0 | 0 | |
| At least one severe AE | 0 | | 0 | 0 | |
| At least one serious AE | 0 | | 0 | 0 | |
| Death | 0 | | 0 | 0 | |
| At least one AE related | 4 | (66.7) | 6 (75.0) | 2 | (33.3) |
| to LP | | | | | |

AE, adverse event;
LP, lumbar puncture;
PBO, placebo;
PY, patient years

TABLE 9e
Pooled adverse events by event type for
cohorts 1-3 as of September 20, 2023

| | mivelsiran | | mivelsiran | mivelsiran | |
|---|---|---|---|---|---|
| | 25 mg or | | 50 mg or | 75 mg or | |
| | PBO | | PBO | PBO | |
| Patients with | (N = 6, | | (N = 8, | (N = 6, | |
| events, n (%)* | PY = 7.4) | | PY = 4.4) | PY = 4.9) | |
| At least one adverse | 6 | (100.0) | 7 (87.5) | 6 | (100.0) |
| event | | | | | |
| Procedural headache | 3 | (50.0) | 5 (62.5) | 0 | |
| Procedural pain | 1 | (16.7) | 2 (25.0) | 0 | |
| Back pain | 2 | (33.3) | 0 | 1 | (16.7) |
| Presyncope | 0 | | 1 (12.5) | 2 | (33.3) |
| Syncope | 2 | (33.3) | 0 | 1 | (16.7) |

| | | | | |
|---|---|---|---|---|
| Headache | 0 | 3 (37.5) | 1 | (16.7) |
| Nasopharyngitis | 0 | 0 | 2 | (33.3) |

| | | | | |
|---|---|---|---|---|
| Vomiting | 2 | (33.3) | 0 | 0 |

*Only events that occurred in 2 or more patients in the total population are reported.

TABLE 9f
Blinded adverse events as of Sep. 20, 2023

| | Patients with events, n (%) | | | | | |
|---|---|---|---|---|---|---|
| | mivelsiran | | mivelsiran | | mivelsiran | |
| | 25 mg or | | 50 mg or | | 75 mg or | |
| | placebo | | placebo | | placebo | |
| | (N = 6, patient- | | (N = 8, patient- | | (N = 6, patient- | |
| | years = 6.9) | | years = 5.5) | | years = 5.6) | |
| At least one adverse | 6 | (100.0) | 7 | (87.5) | 6 | (100.0) |
| event | | | | | | |
| Related to study drug | 0 | | 1 | (12.5)^{a} | 0 | |
| Related to lumbar | 4 | (66.7) | 6 | (75.0) | 2 | (33.3) |
| puncture | | | | | | |
| Adverse events | | | | | | |
| reported in more than | | | | | | |
| one patient | | | | | | |
| Back pain | 1 | (16.7) | 0 | | 1 | (16.7) |
| Headache | 0 | | 1 | (12.5) | 1 | (16.7) |
| Nasopharyngitis | 0 | | 0 | | 2 | (33.3) |
| Presyncope | 0 | | 1 | (12.5) | 2 | (33.3) |
| Procedural headache | 3 | (50.0) | 6 | (75.0) | 0 | |
| Procedural pain | 2 | (33.3) | 3 | (37.5) | 0 | |
| Syncope | 2 | (33.3) | 0 | | 1 | (16.7) |
| Vomiting | 2 | (33.3) | 0 | | 0 | |
| At least one moderate | 4 | (66.7) | 5 | (62.5) | 4 | (66.7) |
| adverse event | | | | | | |
| At least one serious | 0 | | 0 | | 0 | |
| adverse event | | | | | | |

^{a}One patient in the 50 mg/placebo cohort had two mild adverse events (post-lumbar puncture headache and nausea); each event was deemed both drug-related and procedure-related by the investigator and resolved on the same day. No other drug-related adverse events were reported.

TABLE 10
Mean percent changes from baseline for sAPPα
and sAPPβ at Day 15 in Cohorts 1 and 2

| | % Change from | | % Change from | |
|---|---|---|---|---|
| | Baseline sAPPα | | Baseline sAPPβ | |
| | Mean (range) | | Mean (range) | |
| Placebo (N = 4) | 19 | (16, 21) | −1 | (−8, 6) |
| 25 mg (N = 4) | 15 | (0, 25) | −4 | (−55, 28) |
| 75 mg (N = 4) | −55 | (−71, −21) | −69 | (−83, −37) |

TABLE 11
Changes in CSF sAPPα and sAPPβ Levels at Day 15, Month 6, and Month 10^{a}

| | mivelsiran | | mivelsiran | mivelsiran | | | |
|---|---|---|---|---|---|---|---|
| | 25 mg | | 50 mg | 75 mg | | Placebo | |
| | (N = 4) | | (N = 6) | (N = 4) | | (N = 6) | |
| SAPPα | | | | | | | |
| Day 15, patients with data | n = 4 | | n = 6 | n = 4 | | n = 5 | |
| Mean (SEM) change from | 15.4 | (5.6) | −56.4 (4.2) | −54.7 | (11.5) | −8.3 | (11.2) |
| baseline, % | | | | | | | |
| Month 6, patients with data | n = 4 | | n = 5 | n = 4 | | n = 6 | |
| Mean (SEM) change from | −4.2 | (20.1) | −31.3 (7.6) | −55.5 | (7.5) | 4.0 | (20.0) |
| baseline, % | | | | | | | |
| Month 10, patients with data | n = 1 | | n = 0 | n = 4 | | n = 0 | |
| Mean (SEM) change from | — | | — | −34.4 | (4.4) | — | |
| baseline, % | | | | | | | |
| SAPPβ | | | | | | | |
| Day 15, patients with data | n = 4 | | n = 6 | n = 4 | | n = 5 | |
| Mean (SEM) change from | −4.4 | (18.3) | −71.3 (4.1) | −68.8 | (10.6) | −5.3 | (4.6) |
| baseline, % | | | | | | | |
| Month 6, patients with data | n = 4 | | n = 5 | n = 4 | | n = 6 | |
| Mean (SEM) change from | 7.1 | (13.1) | −47.8 (5.5) | −65.1 | (9.2) | 11.5 | (11.7) |
| baseline, % | | | | | | | |
| Month 10, patients with data | n = 1 | | n = 0 | n = 4 | | n = 0 | |
| Mean (SEM) change from | — | | — | −38.8 | (8.2) | — | |
| baseline, % | | | | | | | |

^{a}Time points with an n of ≤2 are not included.
CSF, cerebrospinal fluid; sAPP, soluble amyloid precursor protein; SEM, standard error of the mean.

TABLE 12
Changes in CSF Aβ42 and Aβ40 Levels at Month 6

| | mivelsiran | mivelsiran | mivelsiran | | |
|---|---|---|---|---|---|
| | 25 mg | 50 mg | 75 mg | | Placebo |
| | (N = 4) | (N = 6) | (N = 4) | | (N = 6) |
| Aβ42, patients with data | n = 4 | n = 5 | n = 4 | | n = 6 |
| Mean (SEM) change from | −1.8 (1.8) | −37.9 (7.5) | −32.5 | (10.0) | 9.0 (8.4) |
| baseline to Month 6, % | | | | | |
| Aβ40, patients with data | n = 4 | n = 5 | n = 4 | | n = 6 |
| Mean (SEM) change from | −2.6 (6.1) | −47.3 (5.1) | −55.7 | (6.4) | 2.6 (7.2) |
| baseline to Month 6, % | | | | | |

Aβ40, amyloid beta peptide length 40 amino acids; Aβ42, amyloid beta peptide length 42 amino acids; CSF, cerebrospinal fluid; SEM, standard error of the mean.

TABLE 13
Example combinations of % decrease and duration of decrease in a parameter

| | | | % decrease (at least about) | | | | | | | | | | | | |
|---|---|---|---|---|---|---|---|---|---|---|---|---|---|---|---|
| | | | 5% | 10% | 20% | 30% | 40% | 50% | 55% | 60% | 70% | 80% | 90% | 95% | 100% |
| Duration | 1 | week | X | X | X | X | X | X | X | X | X | X | X | X | X |
| of decrease | 2 | weeks | X | X | X | X | X | X | X | X | X | X | X | X | X |
| sustained | 1 | month | X | X | X | X | X | X | X | X | X | X | X | X | X |
| (at least | 2 | months | X | X | X | X | X | X | X | X | X | X | X | X | X |
| about) | 3 | months | X | X | X | X | X | X | X | X | X | X | X | X | X |
| | 4 | months | X | X | X | X | X | X | X | X | X | X | X | X | X |
| | 5 | months | X | X | X | X | X | X | X | X | X | X | X | X | X |
| | 6 | month | X | X | X | X | X | X | X | X | X | X | X | X | X |
| | 9 | months | X | X | X | X | X | X | X | X | X | X | X | X | X |
| | 10 | months | X | X | X | X | X | X | X | X | X | X | X | X | X |
| | 1 | year | X | X | X | X | X | X | X | X | X | X | X | X | X |

We claim:

1. A method for inhibiting the expression of amyloid precursor protein (APP) in a subject having an APP-associated disease in need thereof, comprising
administering to the subject a fixed dose of about 25 mg to about 225 mg of mivelsiran or an equivalent amount of a pharmaceutically acceptable salt thereof (e.g., sodium salt), thereby inhibiting the expression of APP in the subject.

2. A method for treating or preventing an APP-associated disease in a subject in need thereof, the method comprising
administering to the subject a fixed dose of about 25 mg to about 225 mg of mivelsiran or an equivalent amount of a pharmaceutically acceptable salt thereof (e.g., sodium salt), thereby treating or preventing an APP-associated disease in the subject.

3. The method of any one of claims 1-2, wherein the subject is a human.

4. The method of any one of claims 1-3, wherein the APP-associated disorder is Alzheimer's disease (AD).

5. The method of claim 4, wherein the Alzheimer's disease is early-onset Alzheimer's disease (EOAD).

6. The method of any one of claims 1-5, wherein the subject shows a decrease in the level of sAPPα and/or sAPPβ in the cerebral spinal fluid (CSF) following administration of mivelsiran or the equivalent amount of a pharmaceutically acceptable salt thereof (e.g., sodium salt).

7. The method of claim 6, wherein the subject shows an at least about 40% decrease in the level of sAPPα and/or sAPPβ in the cerebral spinal fluid (CSF).

8. The method of claim 6, wherein the subject shows an about 50% to about 90% decrease in the level of sAPPα and/or sAPPβ in the cerebral spinal fluid (CSF).

9. The method of claim 6, wherein the subject shows the decrease in the level of sAPPα and/or sAPPβ in the CSF that is sustained for at least 3 months.

10. The method of claim 9, wherein the subject shows at least about 40% decrease in the level of sAPPα and/or sAPPβ in the CSF that is sustained for at least 3 months.

11. The method of claim 9, wherein the subject shows an at least about 40% decrease in the level of sAPPα and/or sAPPβ in the CSF that is sustained for at least 6 months.

12. The method of claim 9, wherein the subject shows an at least about 40% decrease in the level of sAPPα and/or sAPPβ in the CSF that is sustained for at least 10 months.

13. The method of claim 9, wherein the subject shows an at least about 55% decrease in the level of sAPPα and/or sAPPβ in the CSF that is sustained for at least 3 months.

14. The method of claim 9, wherein the subject shows an at least about 55% decrease in the level of sAPPα and/or sAPPβ in the CSF that is sustained for at least 6 months.

15. The method of claim 9, wherein the subject shows an at least about 55% decrease in the level of sAPPα and/or sAPPβ in the CSF that is sustained for at least 10 months.

16. The method of claim 9, wherein the subject shows an at least about 20% decrease in the level of sAPPα and/or sAPPβ in the CSF that is sustained for at least 12 months.

17. The method of any one of claims 1-16, wherein the subject shows a decrease in Aβ accumulation following administration of mivelsiran or the equivalent amount of a pharmaceutically acceptable salt thereof (e.g., sodium salt).

18. The method of claim 17, wherein the subject shows a decrease in intracellular and/or extracellular Aβ40 and/or Aβ42.

19. The method of any one of claims 1-18, wherein the subject shows a decrease in amyloid plaque formation and/or accumulation.

20. The method of any one of claims 1-19, wherein the subject shows a decrease in a level of a disease biomarker in a body fluid of the subject.

21. The method of claim 20, wherein the body fluid is a cerebrospinal fluid (CSF) or a plasma.

22. The method of claim 20, wherein the body fluid is the CSF, and the disease biomarker in the CSF that is decreased is selected from the group consisting of Aβ40, Aβ42, neurofilament light chain protein (NfL), neurofilament heavy chain protein (NfH), apolipoprotein E (APOE), neurogranin, synaptosome associated protein 25 (SNAP25), S100 calcium binding protein B (S100B), t-Tau, and p-Tau.

23. The method of claim 22, wherein the disease biomarker in the CSF that is decreased is Aβ42 and/or Aβ40.

24. The method of claim 23, wherein the subject shows an at least about 30% decrease in the level of Aβ42 and/or at least about 50% decrease in the level of Aβ40 in the CSF.

25. The method of claim 23, wherein the subject shows an at least about 40% decrease in the level of Aβ42 and/or at least about 60% decrease in the level of Aβ40 in the CSF of the subject.

26. The method of claim 20, wherein the body fluid is the plasma, and the disease biomarker in the plasma that is decreased is selected from the group consisting of sAPPα, sAPPβ, Aβ40, Aβ42, NfL, t-Tau, and p-Tau.

27. The method of any one of claims 1-26, wherein neurotoxicity or neuroinflammation is not produced or increased following administration of mivelsiran or the equivalent amount of a pharmaceutically acceptable salt thereof (e.g., sodium salt).

28. The method of any one of claims 1-27, wherein a level of an inflammatory biomarker is not increased following administration of mivelsiran or the equivalent amount of a pharmaceutically acceptable salt thereof (e.g., sodium salt).

29. The method of claim 28, wherein the inflammatory biomarker is selected from the group consisting of complement component 3 (C3), complement component 5 (C5), interleukin-6 (IL-6), interleukin-1 (IL-1), tumor necrosis factor (TNF), monocyte chemoattractant protein-1 (MCP-1), and chitinase-3-like protein 1 (YKL-40).

30. The method of any one of claims 8-29, wherein the decrease or the increase is relative to a reference level.

31. The method of claim 30, wherein the reference level is a level observed in the subject prior to the administration, or a level observed in a subject administered a placebo.

32. The method of claim 30, wherein the reference level is a predetermined threshold level.

33. The method of any one of claims 1-33, wherein the administration improves one or more signs and/or symptoms of the APP-associated disorder in the subject.

34. The method of claim 33, wherein the administration improves the subject's score in one or more of Repeatable Battery for the Assessment of Neuropsychological Status (RBANS), Alzheimer's Disease Assessment Scale-Cognitive Subscale 13-item Version (ADAS-Cog13), Clinical Dementia Rating (CDR), Neuropsychiatric Inventory Questionnaire (NPI-Q), Pittsburgh Sleep Quality Index (PSQI), and the Alzheimer's Disease Cooperative Study-Clinical Global Impression of Change (ADCS-CGIC).

35. The method of any one of claims 1-34, wherein the fixed dose of mivelsiran is 25 mg to 75 mg, 30 mg to 75 mg, 35 mg to 75 mg, 40 mg to 75 mg, 45 mg to 75 mg, 50 mg to 75 mg, 25 mg to 60 mg, 30 mg to 60 mg, 35 mg to 60 mg, 40 mg to 60 mg, 45 mg to 60 mg, 50 mg to 60 mg, 25 mg to 50 mg, 30 mg to 50 mg, 35 mg to 50 mg, 40 mg to 50 mg, 45 mg to 50 mg, 25 mg to 100 mg, 50 mg to 100 mg, 75 mg to 100 mg, 25 mg to 150 mg, 30 mg to 150 mg, 35 mg to 150 mg, 40 mg to 150 mg, 45 mg to 150 mg, 50 mg to 150 mg, 75 mg to 150 mg, 100 mg to 150 mg, 50 mg to 75 mg, 75 mg to 100 mg, 100 mg to 150 mg, 150 mg to 225 mg, or an equivalent amount of a pharmaceutically acceptable salt thereof (e.g., sodium salt).

36. The method of claim 35, wherein the fixed dose is about 25 mg to about 100 mg or about 50 mg to about 100 mg or about 75 mg to about 150 mg of mivelsiran or an equivalent amount of a pharmaceutically acceptable salt thereof (e.g., sodium salt).

37. The method of claim 35, wherein the fixed dose is about 75 mg to about 100 mg or about 100 mg to about 150 mg of mivelsiran or an equivalent amount of a pharmaceutically acceptable salt thereof (e.g., sodium salt).

38. The method of any one of claims 1-37, wherein the fixed dose is about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, or about 150 mg of mivelsiran or an equivalent amount of a pharmaceutically acceptable salt thereof (e.g., sodium salt).

39. The method of any one of claims 1-38, wherein mivelsiran or an equivalent amount of a pharmaceutically acceptable salt thereof is administered to the subject as a single dose.

40. The method of any one of claims 1-39, wherein a multiple dose regimen of the fixed dose of mivelsiran or an equivalent amount of a pharmaceutically acceptable salt thereof is administered to the subject.

41. The method of claim 40, wherein a cumulative annual dose of mivelsiran is 450 mg or less, or an equivalent amount of a pharmaceutically acceptable salt thereof (e.g., sodium salt).

42. The method of claim 40 or 41, wherein each dose in the multiple dose regimen is administered at least 3 months apart.

43. The method of claim 40 or 41, wherein each dose in the multiple dose regimen is administered at least 6 months apart.

44. The method of claim 40 or 41, wherein each dose in the multiple dose regimen is administered at least 9 months apart.

45. The method of claim 40 or 41, wherein each dose in the multiple dose regimen is administered at least 12 months apart.

46. The method of any one of claims 1-45, wherein mivelsiran or an equivalent amount of a pharmaceutically acceptable salt thereof (e.g., sodium salt) is administered to the subject intrathecally.

47. The method of any one of claims 1-46, wherein the subject, before administration of mivelsiran or an equivalent amount of a pharmaceutically acceptable salt thereof (e.g., sodium salt), is positive for amyloid by PET upon administration of an amyloid PET agent, optionally wherein the amyloid PET agent is ¹⁸F-labeled.

48. The method of any one of claims 1-47, wherein the CSF of the subject, before administration of mivelsiran or an equivalent amount of a pharmaceutically acceptable salt thereof (e.g., sodium salt): (i) is abnormal for Aβ42, total tau (t-Tau), and phosphorylated tau (p-Tau); (ii) has an abnormal p-Tau/Aβ42 ratio or Aβ42 to p-tau index (PTI); or (iii) has an abnormal t-tau/Aβ42 ratio or Aβ42 to t-tau index (ATI).

49. The method of any one of claims 1-48, wherein the subject, before administration of mivelsiran or an equivalent amount of a pharmaceutically acceptable salt thereof (e.g., sodium salt), scores greater than 20 on a Mini Mental State Examination (MMSE).

50. The method of any one of claims 1-49, wherein the subject, before administration of mivelsiran or an equivalent amount of a pharmaceutically acceptable salt thereof (e.g., sodium salt), has a Clinical Dementia Rating (CDR) Global score of 0.5 or 1.0.

51. The method of any one of claims 1-50, wherein the subject, before administration of mivelsiran or an equivalent amount of a pharmaceutically acceptable salt thereof (e.g., sodium salt), has a clinical diagnosis of mild cognitive impairment or mild dementia due to Alzheimer's disease, and/or has an onset of Alzheimer's disease at an age younger than 65 years.
